(19) United States
(12) Patent Application Publication
(10) Pub. No.: US 2024/0076729 A9
(43) Pub. Date: Mar. 7, 2024

(54) METHOD

(71) Applicant: Oxford Nanopore Technologies PLC, Oxford (GB)

(72) Inventors: Rebecca Victoria Bowen, Oxford (GB); Clive Gavin Brown, Oxford (GB); Mark John Bruce, Oxford (GB); Daniel Ryan Garalde, Oxford (GB); James Edward Graham, Oxford (GB); Andrew John Heron, Oxford (GB); Etienne Raimondeau, Oxford (GB); James White, Oxford (GB); Christopher Peter Youd, Oxford (GB)

(73) Assignee: Oxford Nanopore Technologies PLC, Oxford (GB)

(21) Appl. No.: 18/010,826

(22) PCT Filed: Jun. 18, 2021
(86) PCT No.: PCT/GB2021/051556

§ 371 Date: Dec. 16, 2022

(30) Foreign Application Priority Data
Jun. 18, 2020 (GB) 2009335.7
May 19, 2021 (GB) 2107194.9

(51) Int. Cl. C12Q 1/6869

(52) U.S. Cl. CPC C12Q 1/6869

(57) ABSTRACT

Provided herein is a method of characterising a target polynucleotide as it moves with respect to a nanopore using a motor protein. Also provided are polynucleotide adapters and kits comprising such adapters. The methods, kits and adapters find use in characterising polynucleotides, for example in sequencing.

### RELATED APPLICATIONS

This application is a national stage filing under 35 U.S.C. § 371 of international PCT application PCT/GB2021/051556, filed Jun. 18, 2021, which claims the benefit of Great Britain application number GB 2107194.9, filed May 19, 2021 and Great Britain application number GB 2009335.7, filed Jun. 18, 2020, each of which is herein incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 8, 2022, is named O036670136US00-SEQ-KZM and is 109,186 bytes in size.

### FIELD

The present disclosure provides methods of characterising a target polynucleotide as it moves with respect to a detector such as a transmembrane nanopore. The disclosure also provides novel polynucleotide adapters and kits for use in such methods. The disclosure also provides methods of re-reading a polynucleotide.

### BACKGROUND

Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore.

Polynucleotides are important analytes for sensing in this manner. Nanopore sensing of polynucleotide analytes can reveal the identity and perform single molecule counting of the sensed analytes, but can also provide information on their composition such as their nucleotide sequence, as well as the presence of characteristics such as base modifications, oxidation, reduction, decarboxylation, deamination and more. Nanopore sensing has the potential to allow rapid and cheap polynucleotide sequencing, providing single molecule sequence reads of polynucleotides of tens to tens of thousands bases length.

Two of the essential components of polymer characterization using nanopore sensing are (1) the control of polymer movement through the pore and (2) the discrimination of the component building blocks as the polymer is moved through the pore. During nanopore sensing of analytes such as polynucleotides, it is important to control the movement of the polynucleotide with respect to the pore. Uncontrolled movement can prevent or impede accurate characterisation of the polynucleotides. For example, accurately distinguishing each nucleotide in a homopolymeric polynucleotide is problematic when the movement of the polynucleotide with respect to the pore is not controlled.

It is known to control the movement of a polynucleotide with respect to a detector such as a nanopore by using a motor protein to control the movement of the polynucleotide. Suitable motor proteins include polynucleotide-handling enzymes such as helicases, exonucleases, topoisomerases and the like. The motor protein processes the polynucleotide in a controlled manner. The motor protein can thus be used to control the movement of a polymer such as a polynucleotide with respect to a detector such as a nanopore.

When the detector is a nanopore, disclosed methods typically involve using the motor protein to feed the polynucleotide into the nanopore. This movement direction is described in more detail herein. Methods which involve feeding the polynucleotide into a nanopore have been extensively developed and proven to be very useful in characterising polynucleotides.

However, there remains a need for further methods of characterising polynucleotides. One issue is that in some cases it can be desirable to obtain data different to that obtained from methods which involve feeding polynucleotides into a detector such as a nanopore. For example, the error profiles of data arising from polynucleotide characterisation in methods which involve feeding polynucleotides into a detector can in some circumstances be suboptimal for the accurate characterisation of the polynucleotide. Another issue is that when a motor protein is used to feed a polynucleotide into a detector such as a nanopore, the motor protein may skip forwards in an uncontrolled manner on the polynucleotide strand. This phenomenon is also known as slippage. Slippage can be problematic when characterising polynucleotides as, for example, it can result in one or more nucleotides in the polynucleotide not being accurately characterised. This is particularly problematic when the characterisation of the polynucleotide is to determine its sequence. Strategies for decreasing slippage have to date focused on modifying the motor protein to minimise its propensity to slip on polynucleotide strands. However, alternative methods of moving polynucleotides with respect to detectors such as nanopores which may decrease slippage would also be useful.

There also remains a need for ways of improving the data obtained when characterising polynucleotides. One issue is that in some cases it is desirable to improve the accuracy of the characterisation data obtained when characterising a polynucleotide. In some known methods, a plurality of polynucleotides from a sample of polynucleotides is characterised and the data obtained is aggregated, to improve the overall accuracy. However, this can cause problems. For example, heterogeneity in the sample can mean that when aggregating data obtained from characterising multiple polynucleotide strands, useful information regarding differences between strands can be lost. Furthermore, inefficiencies can arise due to the need to capture a new strand for characterisation once an initial strand has been processed. Alternative and/or improved methods of characterising polynucleotides are thus required.

For these and other reasons there is a need for new and/or improved methods of moving polynucleotides with respect to detectors such as nanopores.

### SUMMARY

The disclosure relates to a method of characterising a target polynucleotide as it moves with respect to a detector, by using a motor protein. More particularly, the disclosure relates to methods in which the motor protein moves the polynucleotide out of the detector. The direction of movement of the polynucleotide is thus opposite to known methods in which the polynucleotide is moved into the nanopore. This is described in more detail herein.

In the disclosed methods, the motor protein is initially stalled on the polynucleotide at a stalling moiety, and the methods provided herein involve destalling the motor protein so that the motor protein can control the movement of the polynucleotide out of the detector (e.g. the nanopore). Methods of stalling and destalling the motor protein are described in more detail herein.

Whilst the disclosure provides nanopores as exemplary detectors, the methods provided herein are amenable to detectors including (i) a zero-mode waveguide, (ii) a field-effect transistor, optionally a nanowire field-effect transistor; (iii) an AFM tip; (iv) a nanotube, optionally a carbon nanotube and (v) a nanopore. The disclosed methods are particularly amenable to methods in which a polynucleotide is moved through a detector or through a structure containing a detector, e.g. a well in a detector chip.

Accordingly, provided herein is a method of characterising a target polynucleotide, the method comprising:

(i) contacting (i) a detector having a first opening and a second opening or (ii) a structure comprising a detector, said structure having a first opening and a second opening with the target polynucleotide; wherein the target polynucleotide has a motor protein stalled thereon; wherein the motor protein is stalled at a stalling moiety;
(ii) contacting the stalling moiety with the nanopore thereby destalling the motor protein; and
(iii) taking one or more measurements characteristic of the target polynucleotide as the motor protein controls the movement of the target polynucleotide through the detector or structure in the direction from the second opening to the first opening; thereby characterising the target polynucleotide.

Also provided is a method of characterising a target polynucleotide, the method comprising:

(i) contacting a detector with the target polynucleotide having a motor protein bound thereto, wherein said target polynucleotide is bound to the motor protein at a polynucleotide binding site of the motor protein;
(ii) taking one or more measurements characteristic of the target polynucleotide as the motor protein controls the movement of the target polynucleotide in a first direction with respect to the detector;
(iii) unbinding the target polynucleotide from the polynucleotide binding site of the motor protein, such that the target polynucleotide moves in a second direction with respect to the detector;
(iv) re-binding the target polynucleotide to the polynucleotide binding site of the motor protein; and taking one or more measurements characteristic of the target polynucleotide as the motor protein controls the movement of the target polynucleotide in the first direction with respect to the detector;
thereby characterising the target polynucleotide.

Also provided herein is a method of characterising a target polynucleotide, the method comprising:

(i) contacting the first opening of a transmembrane nanopore having a first opening and a second opening with the target polynucleotide; wherein the target polynucleotide has a motor protein stalled thereon; wherein the motor protein is stalled at a stalling moiety;
(ii) contacting the stalling moiety with the nanopore thereby destalling the motor protein; and
(iii) taking one or more measurements characteristic of the target polynucleotide as the motor protein controls the movement of the target polynucleotide through the nanopore in the direction from the second opening of the nanopore to the first opening of the nanopore; thereby characterising the target polynucleotide.

In some embodiments, the nanopore spans a membrane having a cis side and a trans side, and the first opening of the nanopore is at the cis side of the membrane and the second opening of the nanopore is at the trans side and the motor protein controls the movement of the target polynucleotide through the nanopore from the trans side to the cis side of the membrane. In some embodiments, the nanopore spans a membrane having a cis side and a trans side, and the first opening of the nanopore is at the trans side of the membrane and the second opening of the nanopore is at the cis side and the motor protein controls the movement of the target polynucleotide through the nanopore from the cis side to the trans side of the membrane.

In some embodiments, the method comprises applying a force across the nanopore, and wherein the motor protein controls the movement of the target polynucleotide through the nanopore in the direction opposite to the applied force; wherein said force preferably comprises a voltage potential applied across the nanopore.

In some embodiments, the motor protein is a helicase. In some embodiments, the motor protein is a DNA-dependent ATPase (Dda) helicase.

In some embodiments, an adapter is attached to one or both ends of the target polynucleotide. In some embodiments, the motor protein is stalled on the adapter.

In some embodiments, the nanopore captures a leader sequence at a first end of the target polynucleotide and the motor protein is stalled at a second end of the target polynucleotide or on an adapter attached to the second end of the target polynucleotide.

In some embodiments:
the target polynucleotide is single-stranded;
the target polynucleotide comprises a leader sequence, wherein the leader sequence is located at the first end of the target polynucleotide or is comprised in an adapter attached to the first end of the target polynucleotide; and
the motor protein is stalled at the second end of the target polynucleotide or is stalled on an adapter at the second end of the target polynucleotide.

In some embodiments the target polynucleotide is double stranded.

In some embodiments:
the target polynucleotide is double-stranded and comprises a first strand and a second strand;
the target polynucleotide comprises a leader sequence, wherein the leader sequence is located at a first end of the polynucleotide and is comprised in the first strand or is comprised in an adapter attached to the first strand; and
the motor protein is stalled at a second end of the target polynucleotide.

In some embodiments the motor protein is stalled at the second end of the first strand of the target polynucleotide or is stalled on an adapter at the second end of the first strand of the target polynucleotide. In some embodiments the first strand and the second strand are attached together by a hairpin adapter at the second end of the first strand; and the motor protein is stalled at the hairpin adapter. In some embodiments the first strand and the second strand are attached together by a hairpin adapter attached to (i) the second end of the first strand and (ii) a first end of the second strand; and the motor protein is stalled at a second end of the second strand or is stalled on an adapter at the second end of the second strand of the double-stranded polynucleotide.

In some embodiments, the target polynucleotide comprises a portion which is complementary to a tag sequence. In some embodiments, the target polynucleotide comprises a portion having an oligonucleotide hybridised thereto, and wherein the oligonucleotide comprises: (a) a hybridising portion for hybridising to the target polynucleotide and (b) (i) a portion complementary to a tag sequence or (ii) an affinity molecule capable of binding to a tag. In some embodiments, the target polynucleotide is double stranded and the portion which is complementary to a tag sequence is a portion of the first strand of the polynucleotide and/or the portion having an oligonucleotide hybridised thereto is a portion of the first strand of the polynucleotide.

In some embodiments, the motor protein is stalled at a stalling site comprising one or more stalling units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
spacer units selected from nitroindoles, inosines, acridines, 2-aminopurines, 2-6-diaminopurines, 5-bromo-deoxyuridines, inverted thymidines (inverted dTs), inverted dideoxy-thymidines (ddTs), dideoxy-cytidines (ddCs), 5-methylcytidines, 5-hydroxymethylcytidines, 2′-O-Methyl RNA bases, Iso-deoxycytidines (Iso-dCs), Iso-deoxyguanosines (Iso-dGs), C3 (OC₃H₆OPO₃) groups, photo-cleavable (PC) [OC₃H₆—C(O)NHCH₂—C₆H₃NO₂—CH(CH₃)OPO₃] groups, hexandiol groups, spacer 9 (iSp9) [(OCH₂CH₂)₃OPO₃] groups, spacer 18 (iSp18) [(OCH₂CH₂)₆OPO₃] groups; and thiol connections; and
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups.

In some embodiments, destalling the motor protein comprises applying a destalling force to the polynucleotide, wherein the destalling force is lower in magnitude and/or of opposite direction to a read force, wherein the read force is the force applied whilst the motor protein controls the movement of the target polynucleotide and the measurements to determine one or more characteristics of the polynucleotide are taken. In some embodiments, destalling the motor protein comprises stepping the applied force one or more times between the destalling force and the read force.

In some embodiments, the motor protein is stalled at a stalling site comprising one or more stalling units and one or more pausing moieties; and wherein contacting the one or more pausing moieties with the nanopore retards the movement of the polynucleotide through the nanopore thereby causing the motor protein to destall from the one or more stalling units. In some embodiments, the pausing moiety comprises one or more pausing units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups; and
a polynucleotide binding protein.

In some embodiments, the target polynucleotide comprises a blocking moiety to prevent the motor protein from disengaging from the polynucleotide. In some embodiments, the target polynucleotide comprises a leader sequence at a first end of the target polynucleotide and the motor protein is stalled at a second end of the target polynucleotide or on an adapter attached to the second end of the target polynucleotide; and the blocking moiety is positioned between the motor protein and the second end of the polynucleotide thereby preventing the motor protein from disengaging from the target polynucleotide at the second end of the target polynucleotide.

Also provided is a polynucleotide adapter having a first end comprising an attachment point for attaching to a double-stranded polynucleotide analyte, and a second end; wherein said polynucleotide adapter comprises (i) a motor protein stalled thereon in an orientation for processing the adapter in the direction of the attachment point, and (ii) a blocking moiety positioned between the motor protein and the second end of the adapter.

Also provided is a kit, comprising a first adapter as described herein and a second adapter comprising a single-stranded leader sequence at a first end and an attachment point for attaching to a double-stranded polynucleotide analyte at a second end.

In some embodiments of the polynucleotide adapter or kit provided herein, said polynucleotide adapter, said motor protein and/or said blocking moiety is as defined herein.

### BRIEF DESCRIPTION OF THE FIGURES

FIG. 1. A schematic showing the distinction between (A) the direction of movement of a polynucleotide (PN) out of a nanopore under the control of a motor protein in accordance with the methods provided herein, as opposed to (B) the movement of a polynucleotide into the pore in contrasting methods. Open arrows show direction of translocation of the motor protein (MP) and PN. In both cases the MP is for example a 5′-3′ helicase.

FIG. 2. A schematic of an embodiment of the methods provided herein, wherein the target polynucleotide is single-stranded; the target polynucleotide comprises a leader sequence located at the first end of the target polynucleotide; and the motor protein is stalled at the second end of the target polynucleotide by a stalling moiety (x). The leader sequence is captured by the nanopore and the single stranded polynucleotide translocates through the nanopore until the stalled motor protein is reached. Once destalled, the motor protein controls the movement of the polynucleotide out of the pore.

FIG. 3. A schematic of an embodiment of the methods provided herein, wherein the target polynucleotide is double-stranded; the target polynucleotide comprises a leader sequence (wavy lines) located at the first end of a first strand of the target polynucleotide; and the motor protein is stalled at a stalling moiety (x) at the second end of the first strand of the target polynucleotide. The leader sequence is captured by the nanopore and the first strand of the target polynucleotide translocates through the nanopore until the stalled motor protein is reached. Once destalled, the motor protein (MP) controls the movement of the first strand of the target polynucleotide (PN) out of the pore.

FIG. 4. A schematic of an embodiment of the methods provided herein, wherein the target polynucleotide is double-stranded; the target polynucleotide comprises a leader sequence (wavy line) located at the first end of a first strand of the target polynucleotide; and the motor protein (MP) is stalled at a stalling moiety (x) at a hairpin adapter connecting the second end of the first strand of the target polynucleotide and a first end of the second strand of the target polynucleotide. The leader sequence is captured by the nanopore and the first strand of the target polynucleotide translocates through the nanopore until the stalled motor protein is reached. Once destalled, the motor protein controls the movement of the first strand of the target polynucleotide (PN) out of the pore.

FIG. 5. A schematic of an embodiment of the methods provided herein, wherein the target polynucleotide is double-stranded; the target polynucleotide comprises a leader sequence located at the first end of a first strand of the target polynucleotide; and a hairpin adapter connects the second end of the first strand of the target polynucleotide and a first end of the second strand of the target polynucleotide. The motor protein (MP) is stalled at a stalling moiety (x) at the second end of the second strand of the target polynucleotide. The leader sequence (wavy line) is captured by the nanopore and the first strand of the target polynucleotide, the hairpin adapter and the second strand of the target polynucleotide translocate through the nanopore until the stalled motor protein is reached. Once destalled, the motor protein controls the movement of the second strand, the hairpin adapter and the first strand of the target polynucleotide (PN) out of the pore.

FIG. 6. Nanopore sequencing adaptor bearing a DNA helicase that translocates 5′ to 3′, in which the 3′ strand is preferentially captured by the nanopore. The adaptor comprises two oligonucleotides known as top strand (A) and bottom strand (B). Top strand comprises: 5′ biotin moiety (C) in complex with monovalent traptavidin (D); DNA motor (with directionality 5′-3′) loaded on closed on poly(dT) binding site (E), and stalled by internal spacer 18 moiety (F); 3′ dT base for ligation to dA-tailed duplex strand (G). Bottom strand comprises: 5′ phosphate moiety (H); region of duplex containing BNA bases as stalling chemistry (I); twenty consecutive 3′-terminal thymidine bases, as leader (wavy line, J); site (K) for hybridizing hydrophobic tether. See example 1.

FIG. 7. Cartoon showing ligation of sequencing adaptors (A) from FIG. 6 to both ends of a dA-tailed double-stranded DNA polynucleotide (B) to yield a continuous duplex.

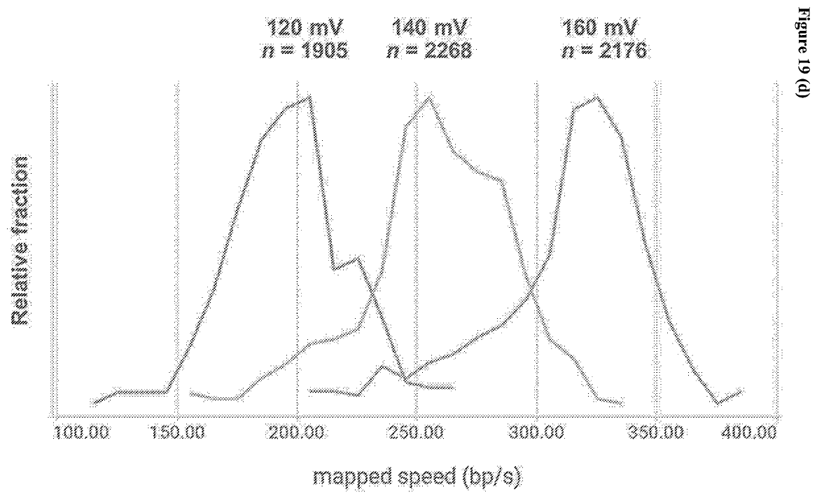

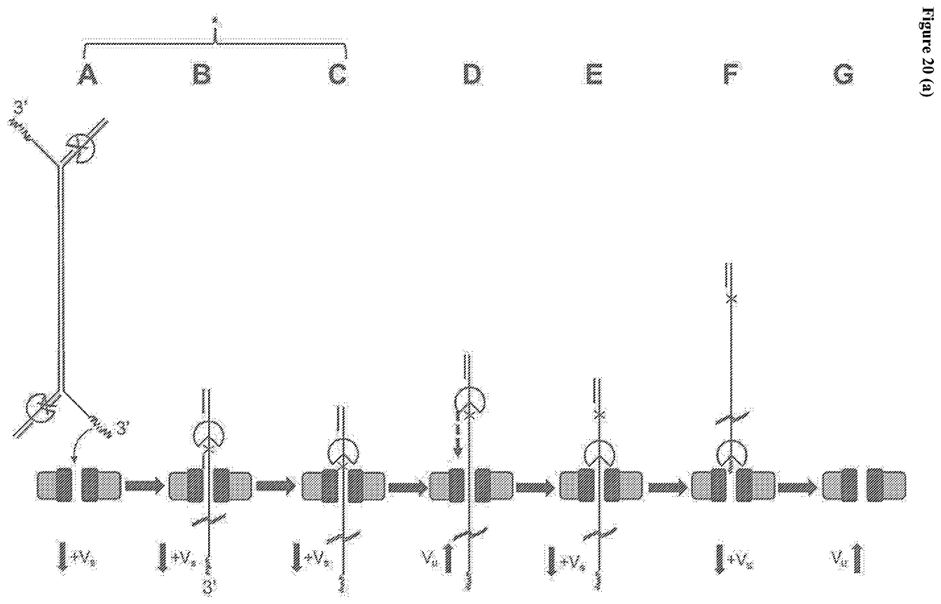

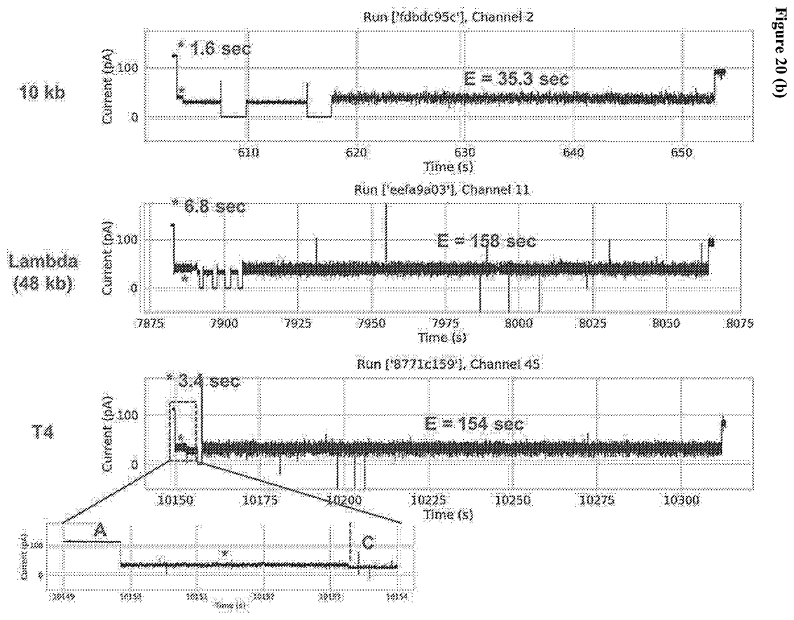

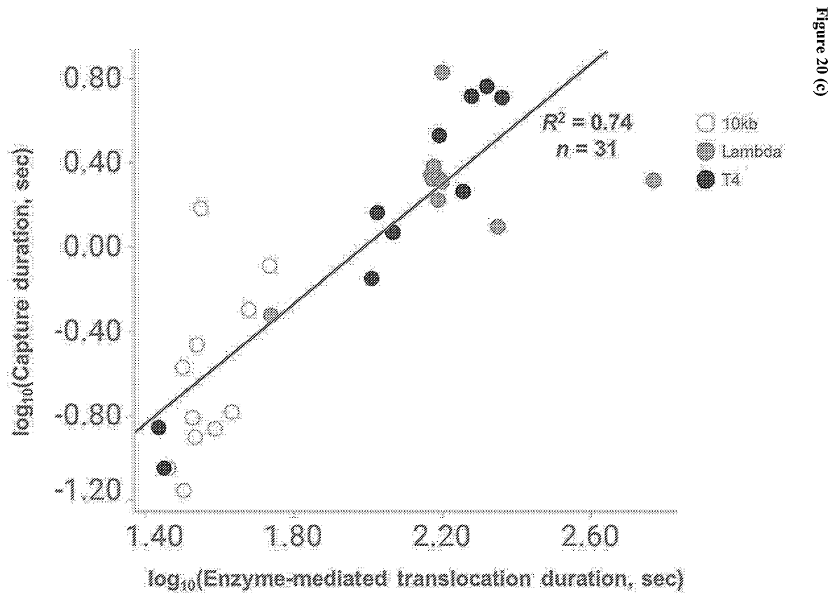

FIG. 8. Experimental schematic of Example 1, showing capture, ‘de-stalling’ and sequencing of a polynucleotide analyte. Vₛ, sequencing potential; Vᵤ, unblock potential. Polarities of applied potential are shown via arrows. The direction of the applied force is the same as the direction of the arrows.
(A) Sequencing potential applied (120 mV). Open pore; capture of polynucleotide analyte (from FIG. 7) via 3′ leader. Separation of duplex via nanopore; complement strand is removed.
(B) Polynucleotide reaches enzyme stalled at spacer moiety. Enzyme cannot move over spacer moiety.
(C) Unblock potential applied (zero mV) such that enzyme is moved away from the nanopore and free to translocate over spacer moiety.
(D) Sequencing potential applied (120 mV). Polynucleotide translocated by nanopore until enzyme reaches nanopore, whereupon enzyme controls movement of polynucleotide out of the nanopore.
(E) DNA motor reaches leader and idles.
(F) Unblock potential applied; DNA motor and analyte ejected from nanopore.

Cycle repeats from (A).

FIG. 9. Top: representative current vs. time trace for Example 1. States A-F correspond to those described in FIG. 8. Bottom: expansion of the boxed region (1 second) shown in the top trace, showing controlled movement of polynucleotide out of nanopore. Applied potentials as follows: A, B: 120 mV; C: 0 mV; D, E and F: 120 mV; cycle repeats.

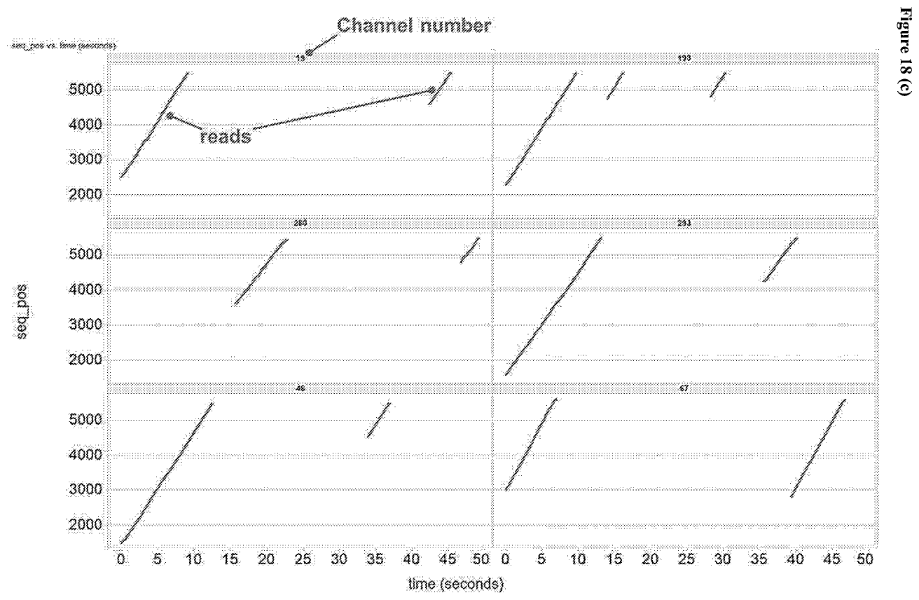

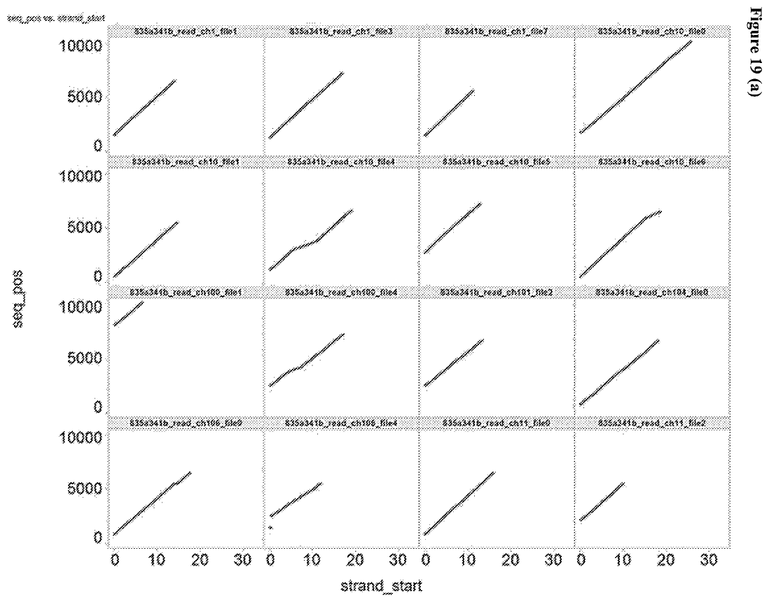

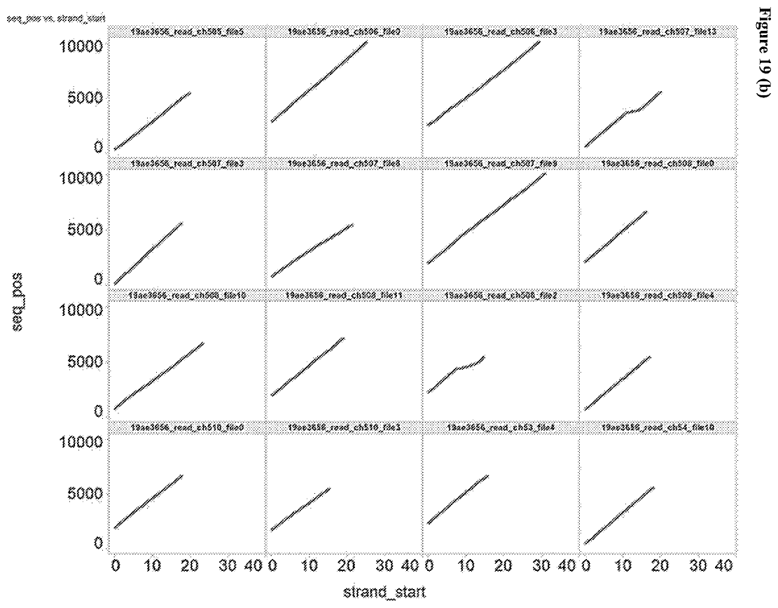

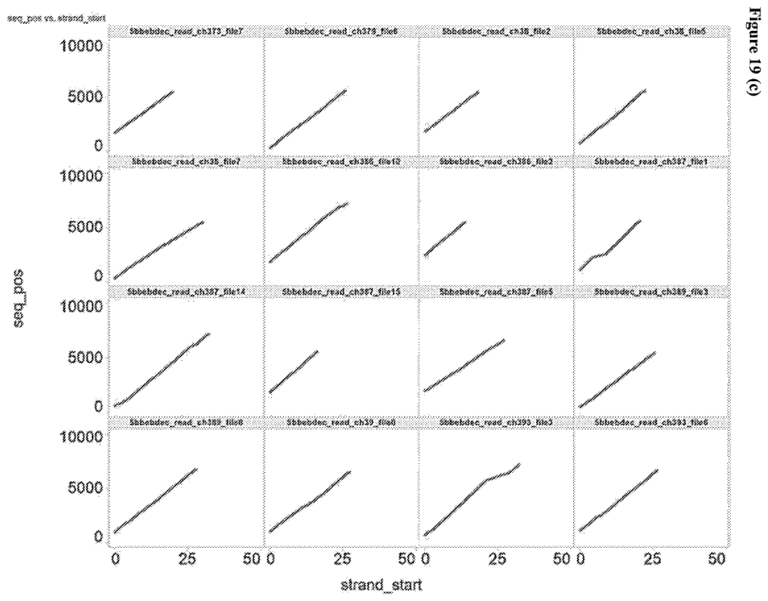

FIG. 10. Components of the experiment described in Example 2 in which both strands of a polynucleotide analyte are first translocated without enzyme through a nanopore; then the enzyme is ‘de-stalled’; then the enzyme controls the movement of both strands of the polynucleotide analyte out of the nanopore.
A. Adapter containing a hairpin moiety and 3′-TCCT overhang that ligates specifically to one end of the polynucleotide analyte.
B. Sequencing adapter, identical to that described in Example 1 and FIG. 6.
C. Polynucleotide analyte bearing asymmetrical ends, one with 3′ dA-tail and one with 3′-AGGA overhang. Template and complement strands are denoted by dashed and solid lines respectively.
D. Ligation of A, B and C yields the library molecule D.

FIG. 11. Experimental schematic of Example 2, showing capture, ‘de-stalling’ and sequencing of both strands of a polynucleotide analyte. Vₛ, sequencing potential; Vᵤ, unblock potential. Polarities of applied potential (if non-zero) are shown via arrows. The direction of the applied force is the same as the direction of the arrows.
(A) Sequencing potential applied (120 mV). Open pore; capture of polynucleotide analyte (from FIG. 7) via 3′ leader. Separation of duplex via nanopore; template and complement strand are translocated into the trans compartment.
(B) Polynucleotide reaches enzyme stalled at spacer moiety. Enzyme cannot move over spacer moiety.
(C) Unblock potential applied (variable, 0 mV to −120 mV) such that enzyme is moved away from the nanopore and free to translocate over spacer moiety.
(D) Sequencing potential applied (120 mV). Polynucleotide translocated by nanopore until enzyme reaches nanopore, whereupon enzyme controls movement of polynucleotide out of the nanopore.
(E) DNA motor moves over template portion and reaches hairpin.
(F) DNA motor moves over complement portion; template and complement strands refold in the cis compartment. Motor reaches leader section and idles on nanopore.

Unblock potential applied; DNA motor and analyte ejected from nanopore.

FIG. 12. (a) Representative current-time traces for the data from Example 2, with the destalling voltage varied between 0 and −120 mV. No events were seen when the eject potential was increased above −60 mV, suggesting that the hairpin formed in trans confers resistance to ejection of the strand up to this voltage. The portion of controlled movement is shown enclosed by a dashed box. (b) Representative current-time trace for an event described in Example 2. States A-G correspond to those described in FIG. 11.

FIG. 13. Representative current-time traces for Example 3, showing capture of polynucleotide analytes into and controlled movement out of a nanopore. DNA motors were ‘de-stalled’ using an ‘active de-stalling’ process described in Example 3. Asterisk denotes where active de-stall potential was applied, first for 5 sec up five times, then 25 sec up five times, with rest states of 3 sec between de-stall attempts. After the first attempt at 5 sec, the enzyme is de-stalled and controls the movement out the polynucleotide out of the nanopore, with the template (Temp.) and complement (Comp.) sections seen, followed by the leader state, per Examples 1 and 2. A: Current-time trace showing the behaviour of a ‘1D DNA library’ similar to that described in Example 1, which is de-stalled after the first attempt. B: Current-time trace showing the behaviour of a joined template-complement polynucleotide (‘2D DNA library’) similar to that described in Example 2 joined by a hairpin moiety, which is de-stalled after the fourth attempt.

FIG. 14. Hairpin moieties of the experiment described in Example 4 in which both strands of a polynucleotide analyte are first translocated without enzyme through a nanopore; then the enzyme is ‘de-stalled’; then the enzyme controls the movement of both strands of the polynucleotide analyte out of the nanopore. Additional moieties in the hairpin introduce an additional signal during the initial enzyme-free capture phase. These moieties are depicted in the figure as follows:

(A) No moiety in hairpin, as control.

(B) Hairpin with oligonucleotide i hybridized to hairpin loop

(C) Three consecutive fluorescein-dT bases ii in hairpin loop, denoted by star

(D) per (C), but with oligonucleotide i hybridized to hairpin loop

FIG. 15. Schematic showing the capture and enzyme-free translocation of a double-stranded polynucleotide analyte bearing a hairpin moiety, in which the hairpin moiety optionally carries a bulky fluorophore and optionally an oligonucleotide hybridized to the hairpin loop. The schematic shows two additional detectable intermediates, A1 and A2, corresponding to the oligonucleotide hybridized to the hairpin loop atop the nanopore, with the fluorophore in the lumen of the nanopore, and to the fluorophore in the lumen of the nanopore alone. An additional state, D1, corresponds to the fluorophore in the lumen of the nanopore, and the enzyme moving over the fluorophore.

FIG. 16.

(a) Data showing the identification of enzyme-free movement of a polynucleotide whose template and complement strands are joined through a hairpin moiety. The polynucleotide is guided through a nanopore via an applied potential prior to the enzyme-controlled movement step. The experimental schematic is similar to that described in Example 2 and FIG. 11. The hairpin is that described in FIG. 14A. (i) Polynucleotide library ligated to sequencing adapter and hairpin adapter containing DNA only. (ii) Representative current-time trace for the molecule shown in (i). Assignment of components A-G is based on the states A-G described in FIG. 11. (iii) Expanded view of the boxed region shown in (ii), showing identification of open pore level A and stall level B. The asterisked region, which has a different shape and noise to B, and also by relation to the other representative molecules described in this Example, is presumed to arise from the enzyme-free translocation portion.
(b) Data showing the identification of enzyme-free movement of a polynucleotide whose template and complement strands are joined through a hairpin moiety, where an oligonucleotide is hybridised to the hairpin. The polynucleotide is guided through a nanopore via an applied potential prior to the enzyme-controlled movement step. The experimental schematic is similar to that described in Example 2 and FIG. 11. The hairpin is that described in FIG. 14B. (i) Polynucleotide library ligated to sequencing adapter and hairpin adapter containing DNA with oligonucleotide (ON) hybridised thereto. (ii) Representative current-time trace for the molecule shown in (i). Assignment of components A-G is based on the states A-G described in FIG. 11. (iii), Expanded view of the boxed region shown in (ii), showing identification of open pore level A and stall level B. An additional level A2 (described in FIG. 15) arises from the hybridized oligonucleotide, when compared to the example shown in FIG. 16a. Thus, the asterisked region corresponds to enzyme-free translocation.
(c) Data showing the identification of enzyme-free movement of a polynucleotide whose template and complement strands are joined through a hairpin moiety, where a three bulky groups (three consecutive fluorescein-dT bases; FAM) are present in the hairpin. The polynucleotide is guided through a nanopore via an applied potential prior to the enzyme-controlled movement step. The experimental schematic is similar to that described in Example 2 and FIG. 11. The hairpin is that described in FIG. 14, C. (i) Polynucleotide library ligated to sequencing adapter and hairpin adapter containing fluorescein bases. (ii) Representative current-time trace for the molecule shown in (i). Assignment of components A-G is based on the states A-G described in FIG. 11. An additional level D1 is presumed to arise through slow movement of the enzyme over the bulky FAM region. (The complement region E is curtailed owing to the eject phase G, so state F is not seen in this example). (iii) Expanded view of the boxed region shown in (ii), showing identification of open pore level A and stall level B. An additional down-tick current level A1 of ˜20 pA (described in FIG. 15) arises from the FAM groups, when compared to the example shown in FIG. 16a. Thus, the asterisked region corresponds to enzyme-free translocation.
(d) Data showing the identification of enzyme-free movement of a polynucleotide whose template and complement strands are joined through a hairpin moiety, where a three bulky groups (three consecutive fluorescein-dT bases; FAM) are present in the hairpin and an oligonucleotide (ON) is hybridised thereto. The polynucleotide is guided through a nanopore via an applied potential prior to the enzyme-controlled movement step. The experimental schematic is similar to that described in Example 2 and FIG. 11. The hairpin is that described in FIG. 14, D. (i) Polynucleotide library ligated to sequencing adapter and hairpin adapter containing fluorescein bases (FAM), with oligonucleotide (ON) hybridised thereto. (ii) Representative current-time trace for the molecule shown in (i). Assignment of components A-G is based on the states A-G described in FIG. 11. An additional level D1 with current level down-ticks is presumed to arise through slow movement of the enzyme over the bulky FAM region. (iii) Expanded view of the boxed region shown in (ii), showing identification of open pore level A and stall level B. An additional down-tick current level A1 of ˜20 pA (described in FIG. 15) arises from the FAM groups, when compared to the example shown in FIG. 16a and FIG. 16c. The additional level A2 owing to the hybridized ON is also seen, through comparison to FIG. 16b. Thus, the asterisked region corresponds to enzyme-free translocation.
(e) Measurement of the duration of enzyme-free translocation of an E. coli test library. (i) Four representative examples from a random E. coli test library described in Example 4, in which the double-stranded polynucleotide is ligated to a sequencing adapter at one end and a hairpin moiety at the other. The hairpin moiety has an oligonucleotide hybridized thereto. The resultant polynucleotide therefore resembles that of FIG. 16b, except the polynucleotide is of random length. The four examples shown are event-fitted current-time traces, which simplifies the raw data. Level A2 and the enzyme-free portion (denoted by an asterisk) are shown in each example. A threshold of 60 pA (dotted line) was used to demarcate the enzyme-free portion A2. The duration of the asterisked portion was therefore measured between the times the current crosses the 60 pA threshold between open pore level A and oligonucleotide level A2. (ii) Relationship between enzyme-controlled strand duration (measured as sum of periods D and E shown in FIG. 16b, ii) and enzyme-free capture duration (measured as described in this Figure, part i), measured for 30 examples, and shown as a scatter plot. A linear regression line is shown with R² value 0.414, demonstrating positive correlation.

FIG. 17.

(a) Nanopore sequencing adaptor bearing a DNA helicase that translocates 5′ to 3′, in which the 3′ strand is preferentially captured by the nanopore. The enzyme is stalled via a separate blocker strand containing a BNA region, and by a spacer moiety on the strand on which the helicase is loaded. The adaptor comprises oligonucleotides known as top strand (A), bottom strand (B), blocker strand (C) and back blocker (D). Blocker strand and back blocker are both hybridized to top strand forming regions of duplex. A DNA motor (with directionality 5′-3′) is loaded on closed on poly(dT) binding site (E) in the single-strand region between C and D, and stalled by internal spacer 18 moiety (F). Top strand bears 3′ dT base for ligation to dA-tailed duplex strand. Bottom strand comprises: 5′ phosphate moiety (circled P); twenty consecutive thymidine bases, as leader (wavy line, G); site (H) for hybridizing hydrophobic tether.
(b) Schematic showing sequencing adapter (A), as described in FIG. 17a, ligated to double-stranded polynucleotide analyte (B) at both ends.
(c) Experimental schematic of Example 5, showing capture, ‘de-stalling’ and sequencing of a polynucleotide analyte. Vₛ, sequencing potential; Vᵤ, unblock potential. Polarities of applied potential are shown via arrows. The direction of the applied force is the same as the direction of the arrows.
(A) Sequencing potential applied (120 mV). Open pore; capture of polynucleotide analyte (from FIG. 7) via 3′ leader. Separation of duplex via nanopore; complement strand is removed.
(B) Nanopore stalls briefly at blocker strand moiety.
(C) Polynucleotide reaches enzyme stalled at spacer moiety. Enzyme cannot move over spacer moiety.
(D) Unblock potential applied (zero mV) such that enzyme is moved away from the nanopore and free to translocate over spacer moiety.
(E) Sequencing potential applied (120 mV). Polynucleotide translocated by nanopore until enzyme reaches nanopore, whereupon enzyme controls movement of polynucleotide out of the nanopore.
(F) DNA motor reaches leader and idles.
(G) Unblock potential applied; DNA motor and analyte ejected from nanopore. Cycle repeats from (A).
   (d) i, Representative current-time trace for Example 5, showing capture of polynucleotide analytes into and controlled movement out of a nanopore, using an adapter in which the biotin-traptavidin back-blocker is replaced with a separate back blocker oligonucleotide, as described in FIGS. 17a and 17b. DNA motors were ‘de-stalled’ using an ‘active de-stalling’ process described in Example 5 and earlier in Example 3. Levels A-G (as described in FIG. 17c) are assigned via relation to previous examples. Boxed regions ii (enzyme-free translocation) and iii (enzyme-controlled translocation) are shown expanded.

FIG. 18.

(a) Experimental schematic of Example 6, showing capture, ‘de-stalling’ and sequencing of both strands of a polynucleotide analyte, with occasional rereading of strands. Vₛ, sequencing potential; Vᵤ, unblock potential. Polarities of applied potential are shown via arrows. The direction of the applied force is the same as the direction of the arrows.
(A) Sequencing potential applied (120 mV). Open pore; capture of polynucleotide analyte (from FIG. 7) via 3′ leader. Separation of duplex via nanopore; template and complement strand are translocated into the trans compartment.
(B) Polynucleotide reaches enzyme stalled at spacer moiety. Enzyme cannot move over spacer moiety.
(C) Unblock potential applied (variable, 0 mV to −120 mV) such that enzyme is moved away from the nanopore and free to translocate over spacer moiety.
(D) Sequencing potential applied (120 mV). Polynucleotide translocated by nanopore until enzyme reaches nanopore, whereupon enzyme controls movement of polynucleotide out of the nanopore.
(E) DNA motor moves over template portion and reaches hairpin.
(F) DNA motor moves over complement portion; template and complement strands refold in the cis compartment. Motor reaches leader section and idles on nanopore.
   State (F) may return to state (E) as the enzyme is pushed from 3′-5′, thus enabling rereading (RR) of the strand.
(G) Unblock potential applied; DNA motor and analyte ejected from nanopore.
   (b) Representative current-time trace from Example 6, showing an example in which the polynucleotide enzyme is read twice via the enzyme being pushed backwards from the C3 leader under applied potential. Enzyme-controlled portions (i) and (ii) are shown expanded and the C3 level also identified.
   (c) Six representative rereading examples from the experiment described in Example 6. The enzyme-controlled portions were mapped using an HMM model trained using data for the pore and enzyme combination used. Reads in the examples shown mapped at least twice to the same strand of a mixture of seven restriction fragments of bacteriophage lambda DNA.

FIG. 19. (a) Representative HMM mapping examples for data described in Example 7, with data collected at a sequencing potential of 120 mV. (b) Representative HMM mapping examples for data described in Example 7, with data collected at a sequencing potential of 140 mV. (c) Representative HMM mapping examples for data described in Example 7, with data collected at a sequencing potential of 160 mV. (d) Histograms of single-molecule enzyme speeds extracted from the data of FIGS. 19a, 19b and 19c. Number of molecules in each population is indicated. Medians of each population as follows: 120 mV, 319 bp/s; 140 mV, 259 bp/s; 160 mV, 196 bp/s.

FIG. 20. (a) Experimental schematic, identical to that shown in FIG. 18a/Example 6. Additionally, the ‘entry’ phase, used to measure enzyme-free translocation (between steps A and C) is marked with an asterisk. (b) Representative current-time traces for three library examples shown in Example 8: a 10 kb PCR fragment (top); bacteriophage lambda DNA (middle); and T4 DNA (bottom). Full-length reads of T4 DNA were not recorded, so an example part-fragment is shown. In each example, the ‘entry’ phase is marked with an asterisk and the enzyme-controlled phase marked E. Durations of each portion were measured by hand and are marked on the traces. An expanded view of the entry phase for the T4 example is shown. It is not possible to reliably detect the portion marked B per FIG. 20a (blocker oligonucleotide atop pore). (c) Log-log scatter plot of the measured capture durations measured from 31 example traces described in Example 8. Markers are coloured in grayscale according to the library from which they were derived.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a motor protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heteropolymer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | |
|---|---|
| Ala | aliphatic, hydrophobic, neutral |
| Cys | polar, hydrophobic, neutral |
| Asp | polar, hydrophilic, charged (−) |
| Glu | polar, hydrophilic, charged (−) |
| Phe | aromatic, hydrophobic, neutral |
| Gly | aliphatic, neutral |
| His | aromatic, polar, hydrophilic, charged (+) |
| Ile | aliphatic, hydrophobic, neutral |
| Lys | polar, hydrophilic, charged (+) |
| Leu | aliphatic, hydrophobic, neutral |
| Met | hydrophobic, neutral |
| Asn | polar, hydrophilic, neutral |
| Pro | hydrophobic, neutral |
| Gln | polar, hydrophilic, neutral |
| Arg | polar, hydrophilic, charged (+) |
| Ser | polar, hydrophilic, neutral |
| Thr | polar, hydrophilic, neutral |
| Val | aliphatic, hydrophobic, neutral |
| Trp | aromatic, hydrophobic, neutral |
| Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

As used herein, an alkylene group is an unsubstituted or substituted bidentate moiety obtained by removing two hydrogen atoms, either both from the same carbon atom, or one from each of two different carbon atoms, of a hydrocarbon compound which may be aliphatic or alicyclic, and is saturated. The hydrocarbon compound may have from 1 to 20 carbon atoms, in which case the alkylene group is a C₁₋₂₀ alkylene. It may for instance have from 1 to 10 carbon atoms in which case the alkylene group is C₁₋₁₀ alkylene. Typically it is C₁₋₆ alkylene, or C₁₋₄ alkylene, for example methylene, ethylene, i-propylene, n-propylene, t-butylene, s-butylene or n-butylene.

An alkenylene group is an unsubstituted or substituted bidentate moiety obtained by removing two hydrogen atoms, either both from the same carbon atom, or one from each of two different carbon atoms, of a hydrocarbon compound which may be aliphatic or alicyclic, and which comprises one or more carbon-carbon double bond. The hydrocarbon compound may have from 2 to 20 carbon atoms, in which case the alkenylene group is a C₂₋₂₀ alkenylene. It may for instance have from 2 to 10 carbon atoms in which case the alkenylene group is C₂₋₁₀ alkenylene. Typically it is C₂₋₆ alkenylene, or C₂₋₄ alkenylene.

An alkynylene group is an unsubstituted or substituted bidentate moiety obtained by removing two hydrogen atoms, either both from the same carbon atom, or one from each of two different carbon atoms, of a hydrocarbon compound which may be aliphatic or alicyclic, and which comprises one or more carbon-carbon triple bond. The hydrocarbon compound may have from 2 to 20 carbon atoms, in which case the alkynylene group is a C₂₋₂₀ alkynylene. It may for instance have from 2 to 10 carbon atoms in which case the alkynylene group is C₂₋₁₀ alkynylene. Typically it is C₂₋₆ alkynylene, or C₂₋₄ alkynylene.

An arylene group is an unsubstituted or substituted monocyclic or fused polycyclic bidentate moiety obtained by removing two hydrogen atoms, one from each of two different aromatic ring atoms of an aromatic compound, which moiety has from 5 to 14 ring atoms (unless otherwise specified). Typically, each ring has from 5 to 7 or from 5 to 6 ring atoms. An arylene group may be unsubstituted or substituted.

A heteroarylene group is a bidentate moiety obtained by removing two hydrogen atoms, one from each of two different ring atoms of a heteroaryl group. A heteroaryl group is a substituted or unsubstituted monocyclic or fused polycyclic (e.g. bicyclic or tricyclic) aromatic group which typically contains from 5 to 14 atoms in the ring portion including at least one heteroatom, for example 1, 2 or 3 heteroatoms, selected from O, S, N, P, Se and Si, more typically from O, S and N. Examples include pyridyl, pyrazinyl, pyrimidinyl, pyridazinyl, furanyl, thienyl, pyrazolidinyl, pyrrolyl, oxadiazolyl, isoxazolyl, thiadiazolyl, thiazolyl, imidazolyl, triazolyl, pyrazolyl, oxazolyl, isothiazolyl, benzofuranyl, isobenzofuranyl, benzothiophenyl, indolyl, indazolyl, carbazolyl, acridinyl, purinyl, cinnolinyl, quinoxalinyl, naphthyridinyl, benzimidazolyl, benzoxazolyl, quinolinyl, quinazolinyl and isoquinolinyl.

A carbocyclylene group, also known as a cycloalkylene group, is a bidentate moiety obtained by removing two hydrogen atoms, one from each of two carbon atoms in a unsubstituted or substituted cyclic alkyl group. Typically, the moiety has from 3 to 10 carbon atoms (unless otherwise specified), including from 3 to 10 ring atoms. Examples include cyclopropane (C3), cyclobutane (C4), cyclopentane (C5), cyclohexane (C6), cycloheptane (C7), methylcyclopropane (C4), dimethylcyclopropane (C5), methylcyclobutane (C5), dimethylcyclobutane (C6), methylcyclopentane (C6), dimethylcyclopentane (C7), methylcyclohexane (C7), dimethylcyclohexane (C8), menthane (C10).

A heterocyclylene moiety is a bidentate moiety obtained by removing two hydrogen atoms from two different ring atoms of a heterocyclyl group. A heterocyclyl group is a unsubstituted or substituted cyclic group which typically contains from 5 to 14 atoms in the ring portion including at least one heteroatom, for example 1, 2 or 3 heteroatoms, selected from O, S, N, P, Se and Si, more typically from O, S and N. Examples include piperazine, piperidine, morpholine, 1,3-oxazinane, pyrrolidine, imidazolidine, oxazolidine, tetrahydropyrazine, tetrahydropyridine, dihydro-1,4-oxazine, tetrahydropyrimidine, dihydro-1,3-oxazine, dihydropyrrole, dihydroimidazole and dihydrooxazole groups.

An arylene-alkylene group is a group formed by forming a bond between an arylene group and an alkylene group as defined herein. A heteroarylene-alkylene group is a group formed by forming a bond between a heteroarylene group and an alkylene group as defined herein. A carbocyclylene-alkylene group is a group formed by forming a bond between an carbocyclylene group and an alkylene group as defined herein. A heterocyclylene-alkylene group is a group formed by forming a bond between group a heterocyclylene group and an alkylene group as defined herein.

When a group is described as being substituted, it is typically substituted by one or more such as 1, 2 or 3, typically 1 or 2, usually 1 substituent. Suitable substituents may be independently selected from halogen; —OR′ and —NR′2 (wherein R′ is typically H or unsubstituted C₁₋₂ alkyl, and unsubstituted C₁ to C₂ alkyl.

### Methods of Characterising Polynucleotides

The disclosure relates to a method of characterising a target polynucleotide as it moves with respect to a detector such as a nanopore, by using a motor protein. Any suitable motor protein can be used in the methods provided herein. Exemplary motor proteins are described in more detail herein.

The disclosure also relates to methods of characterising a target polynucleotide, comprising contacting a detector with the polynucleotide and re-reading the polynucleotide, e.g. as it moves back and forth with respect to the detector. This is described in more detail herein.

More particularly, in some embodiments the disclosure relates to methods in which the motor protein moves the polynucleotide out of the detector (e.g. out of the nanopore). The direction of movement of the polynucleotide in such embodiments is thus opposite to known methods in which the polynucleotide is moved into a nanopore. This is described in more detail herein.

Whilst the disclosure provides nanopores as exemplary detectors, the methods provided herein are amenable to detectors including (i) a zero-mode waveguide, (ii) a field-effect transistor, optionally a nanowire field-effect transistor; (iii) an AFM tip; (iv) a nanotube, optionally a carbon nanotube and (v) a nanopore. The disclosed methods are particularly amenable to methods in which a polynucleotide is moved through a detector or through a structure containing a detector, e.g. a well in a detector chip.

In the disclosed methods, the motor protein is typically initially stalled on the polynucleotide at a stalling moiety. Suitable stalling moieties are described in more detail herein. The stalling of the motor protein on the polynucleotide has various advantages. For example, whilst stalled the motor protein typically consumes less fuel than when unstalled, e.g. when free to move with respect to the polynucleotide. The reduction of this unproductive fuel usage can be advantageous.

The methods provided herein typically involve destalling the motor protein so that the motor protein can control the movement of the polynucleotide out of the detector (e.g. the nanopore). Methods of destalling the motor protein are described in more detail herein. The controlled destalling of the motor protein has various advantages including that the point at which the motor protein starts processing the polynucleotide can be accurately determined. This can be useful in characterising the polynucleotide so that for example no data is lost as a result of undesired movement of the motor protein on the polynucleotide prior to the start of data recordal.

The disclosed methods are based at least in part on the recognition that the data obtained when a polynucleotide is moved out of a detector such as a nanopore can vary from that obtained when the same polynucleotide is moved into the detector (e.g. the nanopore). The data characteristics including signal profile, noise profile, and error profile, can in some embodiments all differ from contrasting methods in which the same polynucleotide is moved into a detector such as a nanopore. In some embodiments the data obtained in the disclosed methods has advantages compared to data obtained in other known methods. The disclosed methods thus increase the options available when polynucleotide characterisation is needed. Users desiring to characterise polynucleotides can thus choose the method best suited to the specific application at issue.

As explained above, the disclosed methods relate in some embodiments to moving a target polynucleotide out of a detector such as a nanopore. Nanopores will be discussed as exemplary detectors herein but the methods are not limited to such.

Nanopores typically have two openings: a first opening and a second opening. Such openings are often referred to as the cis and trans openings of the nanopore. Often the first opening is the cis opening and the second opening is the trans opening, but in some embodiments the first opening is the trans opening and the second opening is the cis opening, respectively. The notation “cis” and “trans” openings in nanopores is routine in the art. For example, the cis opening of a nanopore typically faces the cis chamber of a nanopore device such as an apparatus as described herein having cis and trans chambers, and the trans opening typically faces the trans chamber.

In certain methods provided herein, the first opening of the nanopore is contacted with a polynucleotide having a motor protein stalled thereon. The methods involve using the motor protein to control the movement of the target polynucleotide through the nanopore in the direction from the second opening of the nanopore to the first opening of the nanopore.

From the viewpoint of the motor protein, therefore, the target polynucleotide is moved out of the nanopore. The notation “out” relates to the overall movement of the polynucleotide towards the motor protein. This direction of movement may be contrasted with an alternative mode in which the target polynucleotide is moved by the motor protein “into” the nanopore.

The difference in these movement schemes is profound. In the methods provided herein in which the polynucleotide is moved “out” of the pore, the direction of movement is from the entrance of the nanopore furthest away from the motor protein (i.e. the distal entrance) towards the entrance of the nanopore closest to the motor protein (the proximal entrance). In contrasting methods in which the polynucleotide is moved “into” the pore, the direction of movement is from the entrance of the nanopore closest to the motor protein (the proximal entrance) towards the entrance of the nanopore furthest away from the motor protein (the distal entrance).

In some embodiments of the provided methods, therefore, the nanopore spans a membrane having a cis side and a trans side, and the first opening of the nanopore is at the cis side of the membrane and the second opening of the nanopore is at the trans side. In such embodiments, the motor protein is located on the cis side of the membrane and controls the movement of the target polynucleotide through the nanopore from the trans side to the cis side of the membrane.

In other embodiments of the provided methods the nanopore spans a membrane having a cis side and a trans side, and the first opening of the nanopore is at the trans side of the membrane and the second opening of the nanopore is at the cis side. In such embodiments, the motor protein is located on the trans side of the membrane and controls the movement of the target polynucleotide through the nanopore from the cis side to the trans side of the membrane.

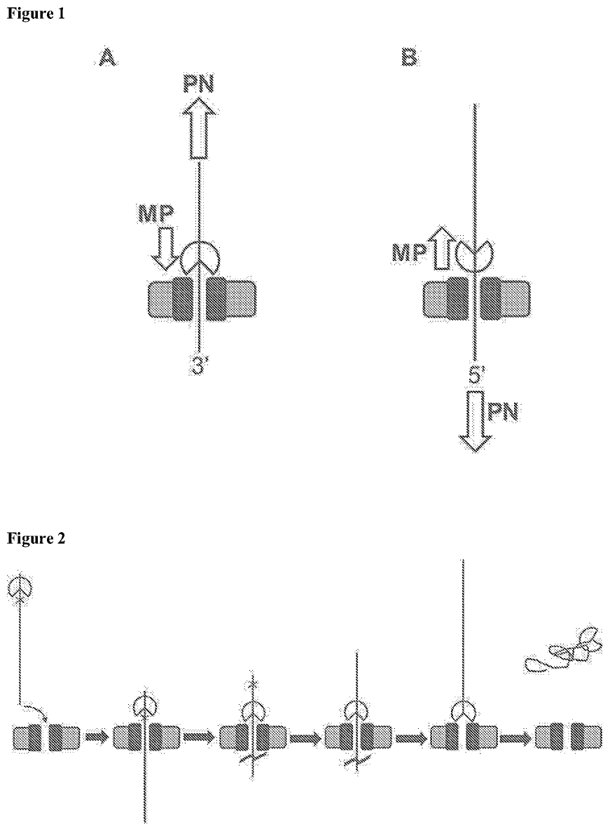

The distinction between the direction of movement of the polynucleotide out of the pore in the methods provided herein, as opposed to the movement of a polynucleotide into the pore in contrasting methods, is illustrated schematically in FIG. 1.

### Re-Reading

In some embodiments, the methods provided herein comprise re-reading the polynucleotide in order to characterise the polynucleotide. Re-reading the polynucleotide comprises taking one or more measurements characteristic of the polynucleotide as the polynucleotide moves back and forth with respect to the detector.

In one embodiment, provided herein is a method of characterising a target polynucleotide, the method comprising:

(i) contacting a detector with the target polynucleotide having a motor protein bound thereto, wherein said target polynucleotide is bound to the motor protein at a polynucleotide binding site of the motor protein;
(ii) taking one or more measurements characteristic of the target polynucleotide as the motor protein controls the movement of the target polynucleotide in a first direction with respect to the detector;
(iii) unbinding the target polynucleotide from the polynucleotide binding site of the motor protein, such that the target polynucleotide moves in a second direction with respect to the detector;
(iv) re-binding the target polynucleotide to the polynucleotide binding site of the motor protein; and taking one or more measurements characteristic of the target polynucleotide as the motor protein controls the movement of the target polynucleotide in the first direction with respect to the detector;

thereby characterising the target polynucleotide.

In a related embodiment, provided herein is a method of characterising a target polynucleotide, the method comprising:

(i) contacting a detector with the target polynucleotide having a motor protein bound thereto, wherein said target polynucleotide is bound to the motor protein at a polynucleotide binding site of the motor protein;
(ii) taking one or more measurements characteristic of the target polynucleotide as the motor protein controls the movement of the target polynucleotide in a first direction with respect to the detector;
(iii) allowing the target polynucleotide to unbind from the polynucleotide binding site of the motor protein, such that the target polynucleotide moves in a second direction with respect to the detector;
(iv) allowing the target polynucleotide to rebind to the polynucleotide binding site of the motor protein; and taking one or more measurements characteristic of the target polynucleotide as the motor protein controls the movement of the target polynucleotide in the first direction with respect to the detector;

thereby characterising the target polynucleotide.

The disclosed methods have many advantages compared to previously known methods. For example, each reading of the target polynucleotide should be of equivalent accuracy as the same strand and same detector moiety is used. This allows for the same basecalling model to be used for each read. It also facilitates combining data from multiple reads. Furthermore, the native sequence is re-read multiple times, allowing (for example) epigenetic information to be retained. The methods are also adaptive: re-reading can be repeated multiple times until data of required accuracy has been obtained.

In more detail, the method may comprise taking one or more measurements characteristic of the target polynucleotide as a motor protein controls the movement of the target polynucleotide in a first direction with respect to the detector. The first direction may be the direction in which the motor protein drives the movement of the polynucleotide. The first direction may be in the direction of a force applied across the detector. The first direction may be in the opposite direction to that of a force applied across the detector.

Often, the detector is comprised in a structure having a first opening and a second opening, or comprises a transmembrane nanopore having a first opening and a second opening; and step (i) comprises contracting the first opening with the target polynucleotide. Typically, the motor protein controls the movement of the target polynucleotide in the direction from the second opening to the first opening. Typically, when the target polynucleotide is unbound from the polynucleotide binding site of the motor protein, the target polynucleotide moves in the direction from the first opening to the second opening.

Accordingly, when the detector is or comprises a nanopore, the first direction may be “into” the nanopore as described herein. Thus, in some embodiments the movement of the polynucleotide whilst one or more measurements are taken is out of the nanopore. In some embodiments the nanopore spans a membrane having a cis side and a trans side; the first opening of the nanopore is at the cis side of the membrane and the second opening of the nanopore is at the trans side; the motor protein is located on the cis side of the membrane and controls the movement of the target polynucleotide through the nanopore from the cis side to the trans side of the membrane. In other embodiments, the nanopore spans a membrane having a cis side and a trans side; the first opening of the nanopore is at the cis side of the membrane and the second opening of the nanopore is at the trans side; the motor protein is located on the trans side of the membrane and controls the movement of the target polynucleotide through the nanopore from the trans side to the cis side of the membrane.

More often, when the detector is or comprises a nanopore, the first direction is “out” of the nanopore as described herein. Thus, in some embodiments the movement of the polynucleotide whilst one or more measurements are taken is out of the nanopore. In some embodiments the nanopore spans a membrane having a cis side and a trans side; the first opening of the nanopore is at the cis side of the membrane and the second opening of the nanopore is at the trans side; the motor protein is located on the cis side of the membrane and controls the movement of the target polynucleotide through the nanopore from the trans side to the cis side of the membrane. In other embodiments, the nanopore spans a membrane having a cis side and a trans side; the first opening of the nanopore is at the cis side of the membrane and the second opening of the nanopore is at the trans side; the motor protein is located on the trans side of the membrane and controls the movement of the target polynucleotide through the nanopore from the cis side to the trans side of the membrane.

The provided methods may comprise unbinding the target polynucleotide from the polynucleotide binding site of the motor protein. This is described in more detail below. Once the target polynucleotide is unbound from the polynucleotide binding site of the motor protein, the target polynucleotide moves in a second direction with respect to the detector. The second direction is typically opposite to the first direction.

Thus, in some embodiments of the methods wherein the detector is or comprises a nanopore, the first direction in which the target polynucleotide moves with respect to the detector is into the nanopore, and the second direction in which the target polynucleotide moves with respect to the detector is out of the nanopore. In other embodiments, the first direction in which the target polynucleotide moves with respect to the detector is out of the nanopore, and the second direction in which the target polynucleotide moves with respect to the detector is into the nanopore.

The provided methods may then comprise re-binding the target polynucleotide to the polynucleotide binding site of the motor protein. The motor protein then controls the movement of the target polynucleotide in the first direction with respect to as one or more measurements characteristic of the polynucleotide are taken. The first direction is the same as the first direction described above.

Thus, in one embodiment, provided herein is a method of characterising a target polynucleotide, the method comprising:

(i) contacting the first opening of a transmembrane nanopore having a first opening and a second opening with the target polynucleotide having a motor protein bound thereto, wherein said target polynucleotide is bound to the motor protein at a polynucleotide binding site of the motor protein;
(ii) taking one or more measurements characteristic of the target polynucleotide as the motor protein controls the movement of the target polynucleotide in the direction from the first opening of the nanopore to the second opening of the nanopore;
(iii) unbinding the target polynucleotide from the polynucleotide binding site of the motor protein, such that the target polynucleotide moves in the direction from the second opening of the nanopore to the first opening of the nanopore;
(iv) re-binding the target polynucleotide to the polynucleotide binding site of the motor protein; and taking one or more measurements characteristic of the target polynucleotide as the motor protein controls the movement of the target polynucleotide in the direction from the first opening of the nanopore to the second opening of the nanopore;

thereby characterising the target polynucleotide. Characterising the target polynucleotide may for example comprise determining the sequence of the target polynucleotide.

For example, in some embodiments the nanopore spans a membrane having a cis side and a trans side, the first opening of the nanopore is at the cis side of the membrane and the second opening of the nanopore is at the trans side, and the motor protein controls the movement of the target polynucleotide through the nanopore from the cis side to the trans side of the membrane. In other embodiments the first opening of the nanopore is at the trans side of the membrane and the second opening of the nanopore is at the cis side and the motor protein controls the movement of the target polynucleotide through the nanopore from the trans side to the cis side of the membrane. In some embodiments the method comprises applying a force (e.g. a voltage potential) across the nanopore, and the motor protein controls the movement of the target polynucleotide through the nanopore in the same direction as the applied force.

In another embodiment, provided herein is a method of characterising a target polynucleotide, the method comprising:

(i) contacting the first opening of a transmembrane nanopore having a first opening and a second opening with the target polynucleotide having a motor protein bound thereto, wherein said target polynucleotide is bound to the motor protein at a polynucleotide binding site of the motor protein;
(ii) taking one or more measurements characteristic of the target polynucleotide as the motor protein controls the movement of the target polynucleotide in the direction from the second opening of the nanopore to the first opening of the nanopore;
(iii) unbinding the target polynucleotide from the polynucleotide binding site of the motor protein, such that the target polynucleotide moves in the direction from the first opening of the nanopore to the second opening of the nanopore;
(iv) re-binding the target polynucleotide to the polynucleotide binding site of the motor protein; and taking one or more measurements characteristic of the target polynucleotide as the motor protein controls the movement of the target polynucleotide in the direction from the second opening of the nanopore to the first opening of the nanopore;

thereby characterising the target polynucleotide. Characterising the target polynucleotide may for example comprise determining the sequence of the target polynucleotide.

For example, in some embodiments the nanopore spans a membrane having a cis side and a trans side, the first opening of the nanopore is at the cis side of the membrane and the second opening of the nanopore is at the trans side, and the motor protein controls the movement of the target polynucleotide through the nanopore from the trans side to the cisside of the membrane. In other embodiments the first opening of the nanopore is at the trans side of the membrane and the second opening of the nanopore is at the cis side and the motor protein controls the movement of the target polynucleotide through the nanopore from the cis side to the trans side of the membrane. In some embodiments the method comprises applying a force (e.g. a voltage potential) across the nanopore, and the motor protein controls the movement of the target polynucleotide through the nanopore in the direction opposite to the applied force.

It is important to distinguish the movement of the polynucleotide in the second direction with respect to the detector from spontaneous slipping that may occur. A slip of one or two bases, for example, is not an example of re-reading as described herein. Typically, in in step (iii) the distance the target polynucleotide moves with respect to the detector is at least 10 nucleotides in length. In some embodiments the distance the target polynucleotide moves with respect to the detector is at least 20 nucleotides in length, e.g. at least 30 nucleotides in length, such as at least 40 nucleotides in length, e.g. at least 50 nucleotides in length, such as at least 100 nucleotides in length. Longer distances may be used. In some embodiments in step (iii) the distance the target polynucleotide moves with respect to the detector is at least 1000 nucleotides (1 kb) in length, such as at least 2 kb, e.g. at least 5 kb or at least 10 kb in length, e.g. at least 100 kb or at least 1000 kb in length.

Steps (iii) and (iv) of the method may be repeated multiple times in order to re-read the target polynucleotide multiple times. Steps (iii) and (iv) may be repeated at least once, such as at least 2 times, such as at least 3 times, e.g. at least 4 times, for example at least 5 times, e.g. at least 10 times, such as at least 20 times, for example at least 50 times, such as at least 100 times, e.g. at least 1000 times, such as at least 10,000 times, e.g. at least 100,000 times or more. Thus, the method may comprise “flossing” the polynucleotide back and forward with respect to the detector.

Thus, if steps (iii) and (iv) are repeated 1 time (and only 1 time) so that the method comprises steps (iii) and (iv) twice and only twice, the method will comprise steps (i), (ii), (iii), (iv), (iii₁), and (iv₁), and measurements characteristic of three portions of the polynucleotide will be taken: a first portion in step (ii); a second portion in step (iii) and (iv); and a third portion in steps (iii₁) and (iv₁). If steps (iii) and (iv) are repeated 2 times (and only 2 times) so that the method comprises steps (iii) and (iv) three times and only three times, the method will comprise steps (i), (ii), (iii), (iv), (iii₁), (iv₁), (iii₂) and (iv₂); and measurements characteristic of four portions of the polynucleotide will be taken: a first portion in step (ii); a second portion in steps (iii) and (iv); a third portion in steps (iii₁) and (iv₁) and a fourth portion in steps (iii₂) and (iv₂). In other words, if steps (iii) and (iv) are repeated n times, then each repeat leads to measurements characteristic of (n+2) portions of the polynucleotide being taken. Repeating steps (iii) and (iv) multiple times can lead to improved characterisation, because the portion of the polynucleotide that is being interrogated by the nanopore is sampled multiple times, and thus any stochastic errors that may be recorded in the analysis become less statistically significant. The accuracy of the characterising data thus obtained can therefore be improved. The methods allow very high accuracy levels to be reached, for example at least 99% accuracy, at least 99.9% accuracy, or at least 99.99% accuracy. Thus, in some embodiments steps (iii) and (iv) are repeated until an accuracy level of at least 99%, such as at least 99.9%, or at least 99.99% accuracy has been reached.

The portion of the polynucleotide which is read in step (ii) and the portion of the polynucleotide which is read in step (iv) of the methods typically overlap. In other words, the method involves re-reading at least a part of the polynucleotide multiple times. Thus, in some embodiments in step (ii) the motor protein controls the movement of a first portion of the target polynucleotide in the first direction with respect to the detector; and in step (iv) the motor protein controls the movement of a second portion of the target polynucleotide in the first direction with respect to the detector; and the first portion at least partially overlaps with the second portion. In some embodiments the second portion overlaps with at least 10%, at least 20%, at least 30%, at least 40%, at least 50%, at least 60%, at least 70%, at least 80%, at least 90%, at least 95%, at least 97%, at least 98% or at least 99% of the first portion. In some embodiments the first portion is the same as the second portion. Thus in some embodiments a portion of the polynucleotide is repeatedly characterised in the provided methods. When at each repeat the second portion of the polynucleotide partially but not wholly overlaps with the first portion of the polynucleotide of the preceding repeat the polynucleotide is ratcheted in a zig-zag manner with respect to the detector. When at each repeat the second portion of the polynucleotide wholly overlaps with the first portion of the polynucleotide of the preceding repeat the same portion of the polynucleotide is flossed back and forwards with respect to the detector.

### Applied Forces During Movement

In some embodiments of the disclosed methods, a force may be applied across the detector, e.g. across the nanopore. The force can be controlled in order to control the methods. For example, by increasing the force the movement of the polynucleotide through the detector (e.g. nanopore) can be increased or decreased, e.g. the rate at which the polynucleotide moves through the pore can be controlled.

In the methods provided herein, any suitable force can be applied. The force may be a potential applied across the detector, for example across the nanopore. In some embodiments, no external force is applied across the nanopore. For example, in some embodiments no electrical potential is applied. Such embodiments are in some embodiments particularly suited to methods in which optical measurements are taken as the polynucleotide moves with respect to the nanopore.

In other embodiments, the force may be a voltage force applied across the nanopore. A voltage force may be applied using any suitable apparatus, such as an apparatus described herein. Suitable voltage potentials are described in more detail herein.

In some embodiments the force is applied across a membrane in which the nanopore is embedded. The force is typically applied from the cis side to the trans side of the membrane; i.e. from the cis side to the trans side of the nanopore. The force may be a positive voltage applied across the nanopore or a negative voltage applied across the nanopore.

Typically the force is a positive voltage applied across the nanopore such that the trans side of the pore is positive relative to the cis side of the pore. In such embodiments, the force thus attracts the negatively charged polynucleotides to move from the cis side to the trans side of the pore. In such embodiments, the methods provided herein typically comprise using the motor protein at the cis side of the pore to control the movement of the polynucleotide in the direction from the trans side of the pore to the cis side of the pore against the applied force; i.e. in the direction opposite to the applied force. However, in some embodiments, the methods provided herein (e.g. methods of re-reading a polynucleotide) may comprise using the motor protein at the cis side of the pore to control the movement of the polynucleotide in the direction from the cis side of the pore to the trans side of the pore in the same direction as the applied force.

In other embodiments the force is a negative voltage applied across the nanopore such that the trans side of the pore is negative relative to the cis side of the pore. In such embodiments, the force thus attracts the negatively charged polynucleotides to move from the trans side to the cis side of the pore. In such embodiments, the methods provided herein typically comprise using the motor protein at the trans side of the pore to control the movement of the polynucleotide in the direction from the cis side of the pore to the trans side of the pore against the applied force; i.e. in the direction opposite to the applied force. However, in some embodiments, the methods provided herein (e.g. methods of re-reading a polynucleotide) may comprise using the motor protein at the trans side of the pore to control the movement of the polynucleotide in the direction from the trans side of the pore to the cis side of the pore in the same direction as the applied force.

As explained below, however, the methods provided herein do not rely on moving the polynucleotide in the direction opposite to an applied force. The direction of movement can in some embodiments be in the same direction as any applied force, whilst still being in the direction out of the pore. In such embodiments, the motor protein typically controls the movement of the polynucleotide out of the pore at a speed greater than that which would arise from the applied force alone.

Accordingly, in some embodiments the force is a positive voltage applied across the nanopore such that the trans side of the pore is positive relative to the cis side of the pore; and the methods may comprise using the motor protein at the trans side of the pore to control the movement of the polynucleotide in the direction from the cis side of the pore to the trans side of the pore with the applied force. In other embodiments, the force is a negative voltage applied across the nanopore such that the trans side of the pore is negative relative to the cis side of the pore; and the methods may comprise using the motor protein at the cis side of the pore to control the movement of the polynucleotide in the direction from the trans side of the pore to the cis side of the pore with the applied force.

### Setup

In some embodiments of the provided methods, a leader sequence is comprised in the target polynucleotide or attached to the polynucleotide. The leader sequence may be captured by the detector (e.g. the nanopore) in the methods provided herein.

Leader sequences are described in more detail herein. Typically, a leader sequence is a single-stranded polynucleotide region without significant secondary structure. For example, the leader sequence typically does not form a hairpin or G-quadruplex and thus is amenable to being captured by a nanopore.

The leader sequence is typically provided at the first end of the polynucleotide or comprised in an adapter attached to the first end of the polynucleotide. Adapters are described in more detail herein.

Typically, a leader sequence is provided at a first end of the polynucleotide (e.g. by being comprised in the first end of the target polynucleotide or by being comprised in a polynucleotide adapter attached to the first end of the target polynucleotide) and the motor protein is stalled at a second end of the target polynucleotide or on an adapter attached to the second end of the target polynucleotide. For example, the leader sequence may be present at the 3′ end of a single-stranded polynucleotide and the motor protein may be located at the 5′ end of the single-stranded polynucleotide. Alternatively, the leader sequence may be present at the 5′ end of a single-stranded polynucleotide and the motor protein may be located at the 3′ end of the single-stranded polynucleotide. This setup allows the first end of the polynucleotide to be captured by the nanopore and threaded through the nanopore e.g. from the first end to the second end. The motor protein at the second end of the polynucleotide typically prevents the polynucleotide from fully translocating the nanopore. In methods provided herein, the motor protein at the second end of the polynucleotide can typically thus control the movement of the polynucleotide out of the nanopore towards the motor protein by processing the polynucleotide in the direction of from the second end to the first end.

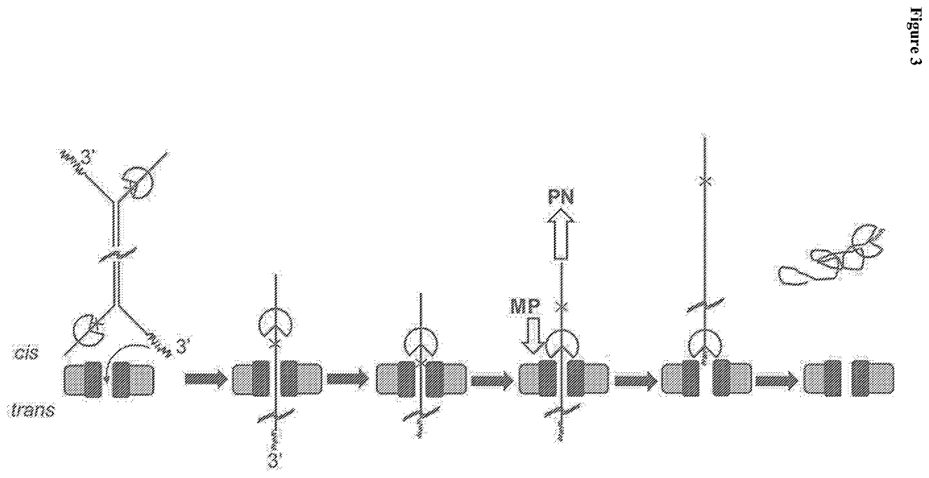

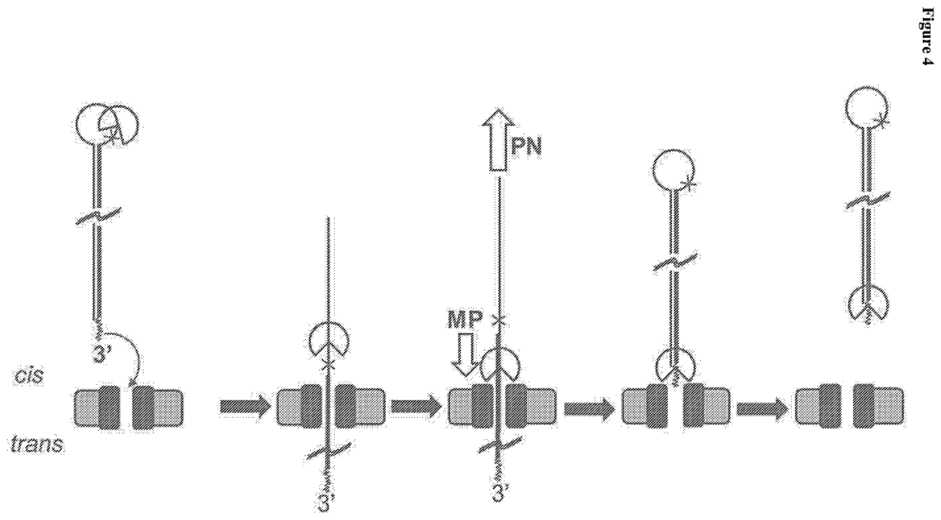

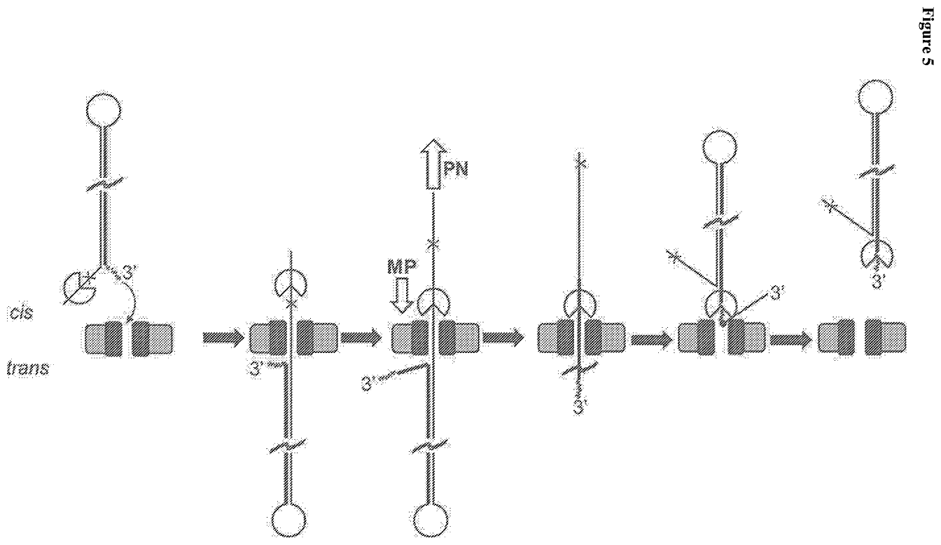

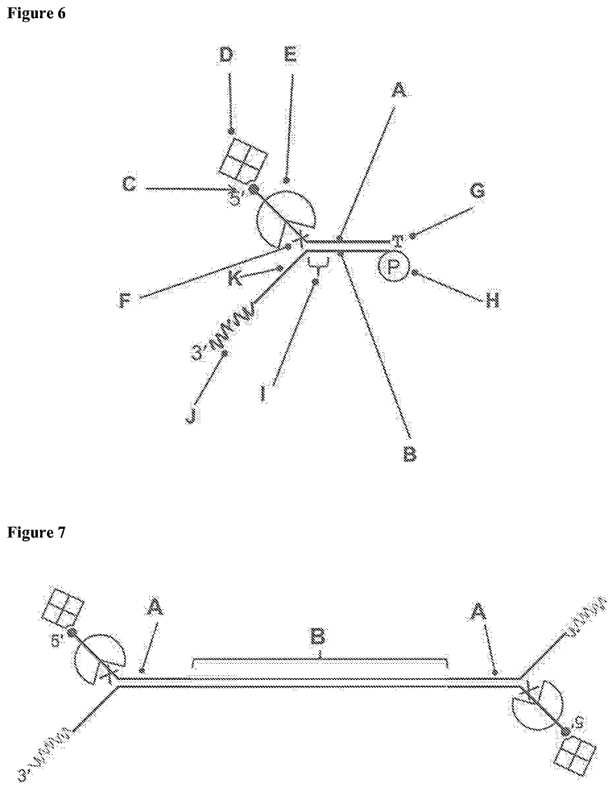

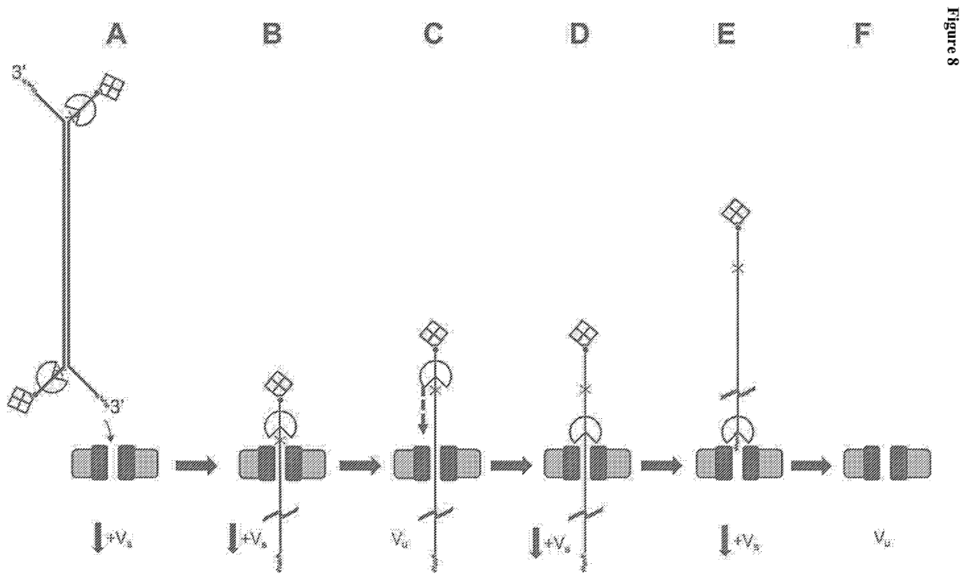

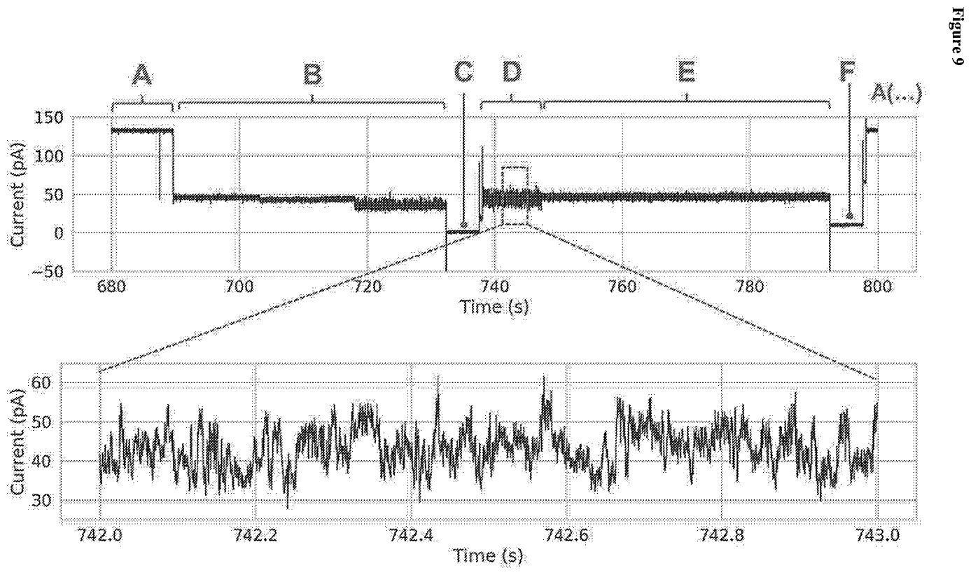

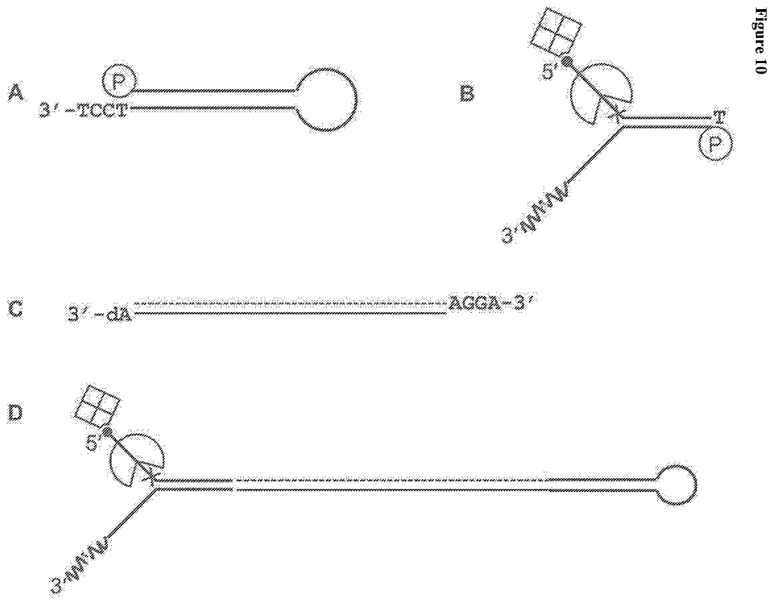

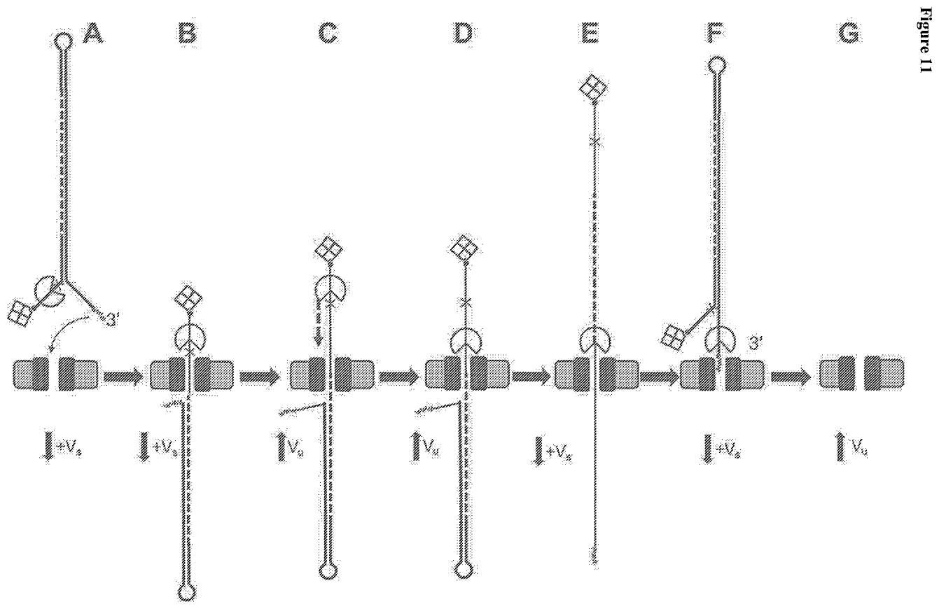

In some embodiments, the target polynucleotide is single-stranded; the target polynucleotide comprises a leader sequence, wherein the leader sequence is located at the first end of the target polynucleotide or is comprised in an adapter attached to the first end of the target polynucleotide; and the motor protein is stalled at the second end of the target polynucleotide or is stalled on an adapter at the second end of the target polynucleotide. In such embodiments, the leader sequence is typically captured by the nanopore and the single stranded polynucleotide translocates through the nanopore until the stalled motor protein is reached. Once destalled, the motor protein controls the movement of the polynucleotide out of the pore. This setup is illustrated schematically in FIG. 2.

In some embodiments, the target polynucleotide is double stranded.

In some embodiments, the target polynucleotide is double-stranded and comprises a first strand and a second strand; the target polynucleotide comprises a leader sequence, wherein the leader sequence is located at a first end of the polynucleotide and is comprised in the first strand or is comprised in an adapter attached to the first strand; and the motor protein is stalled at a second end of the target polynucleotide. This setup allows the first end of the first strand of the double-stranded polynucleotide to be captured by the nanopore and threaded through the nanopore from the first end to the second end. The motor protein at the second end of the polynucleotide typically prevents the polynucleotide from fully translocating the nanopore. The first strand of the double-stranded polynucleotide may be the template strand. The first strand of the double-stranded polynucleotide may be the complement strand.

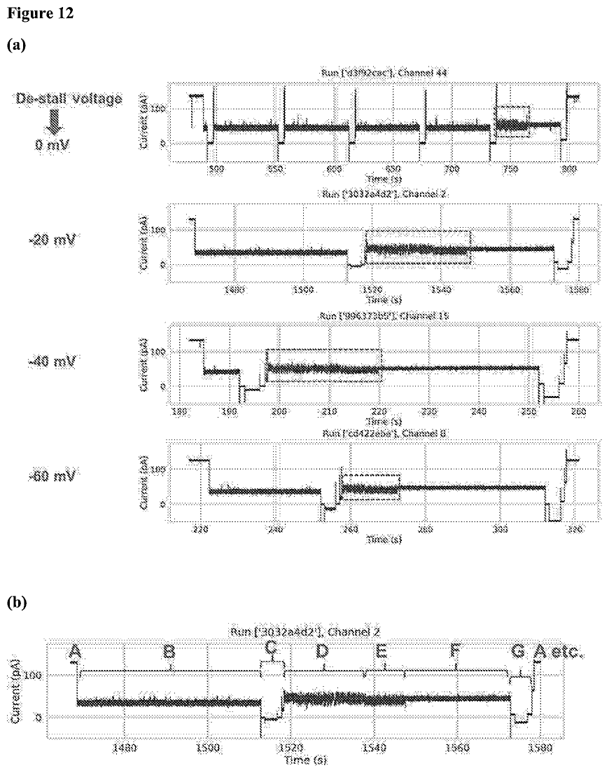

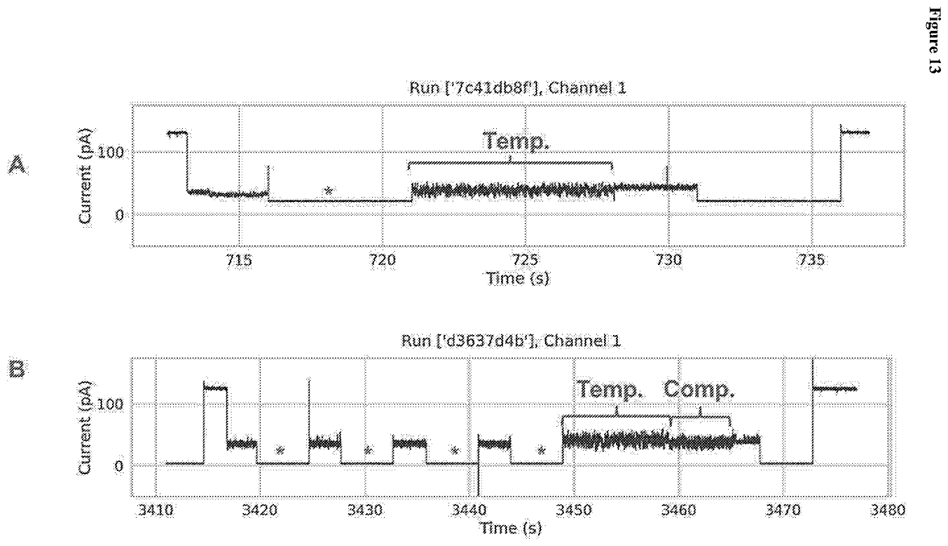

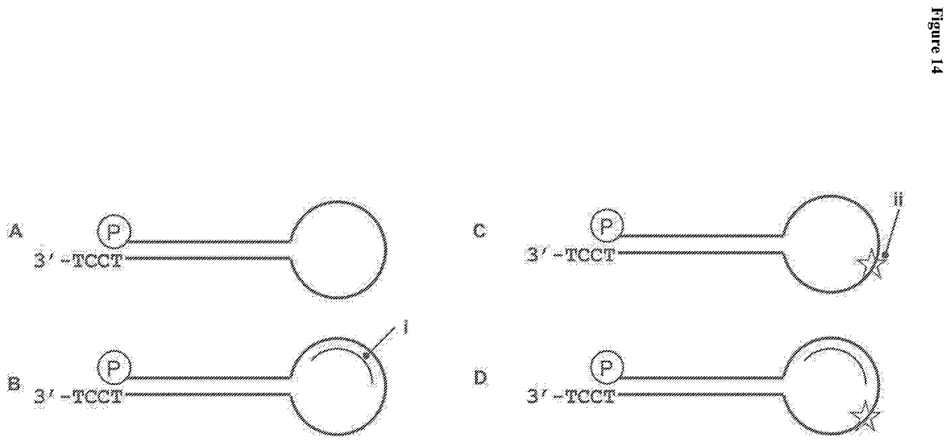

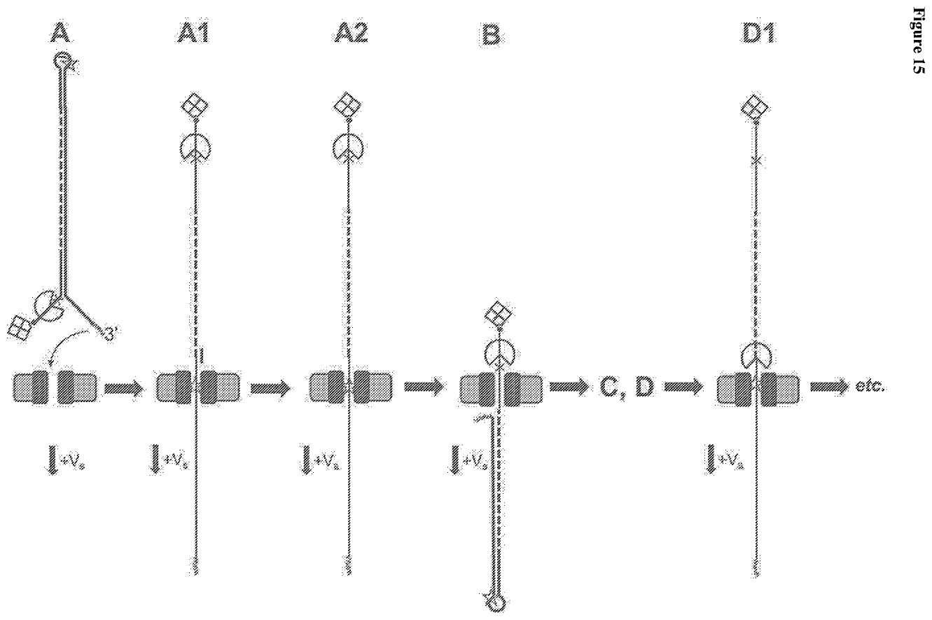

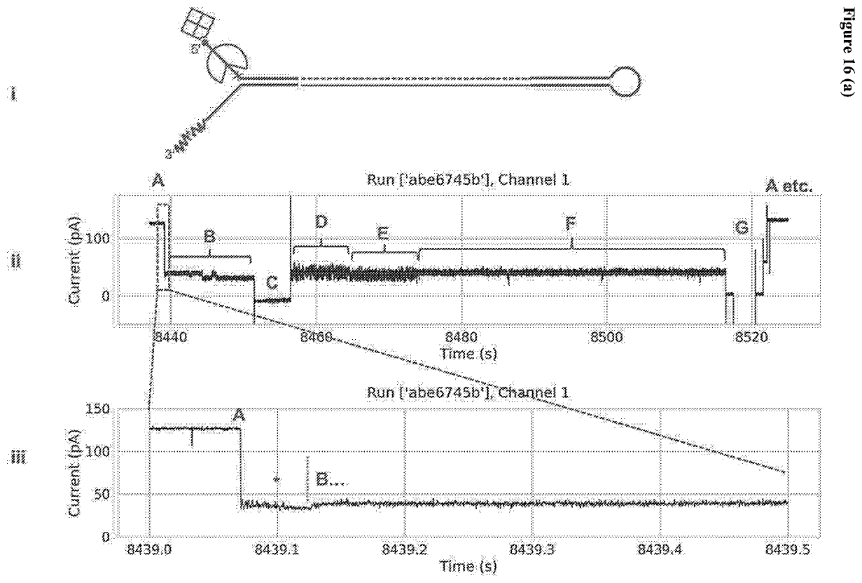

In some embodiments the motor protein is stalled at the second end of the first strand of the target polynucleotide or is stalled on an adapter at the second end of the first strand of the target polynucleotide. In some embodiments the target polynucleotide is double-stranded and comprises a first strand and a second strand; the target polynucleotide comprises a leader sequence, wherein the leader sequence is located at a first end of the polynucleotide and is comprised in the first strand or is comprised in an adapter attached to the first strand; and the motor protein is stalled at the second end of the first strand of the target polynucleotide or is stalled on an adapter at the second end of the first strand of the target polynucleotide. For example, the leader sequence may be present at the 3′ end of the first strand of the double-stranded polynucleotide and the motor protein may be located at the 5′ end of the first strand of the double-stranded polynucleotide. Alternatively, the leader sequence may be present at the 5′ end of the first strand of the double-stranded polynucleotide and the motor protein may be located at the 3′ end of the first strand of the double-stranded polynucleotide. In such embodiments, the leader sequence is typically captured by the nanopore and the single stranded polynucleotide translocates through the nanopore until the stalled motor protein is reached. Once destalled, the motor protein controls the movement of the first strand of the polynucleotide out of the pore. This setup is illustrated schematically in FIG. 3.

In some embodiments the first strand and the second strand are attached together by a hairpin adapter at the second end of the first strand; and the motor protein is stalled at the hairpin adapter. In some embodiments the hairpin adapter is attached at its 5′ end to the 3′ end of the first strand and is attached at its 3′ end to the 5′ end of the second strand of the target double-stranded polynucleotide. In some embodiments the hairpin adapter is attached at its 3′ end to the 5′ end of the first strand and is attached at its 5′ end to the 3′ end of the second strand of the target double-stranded polynucleotide. Accordingly, the hairpin adapter connects the first strand to the second strand. The hairpin adapter typically connects the second end of the first strand of the double-stranded polynucleotide to the first end of the second strand of the double-stranded polynucleotide.

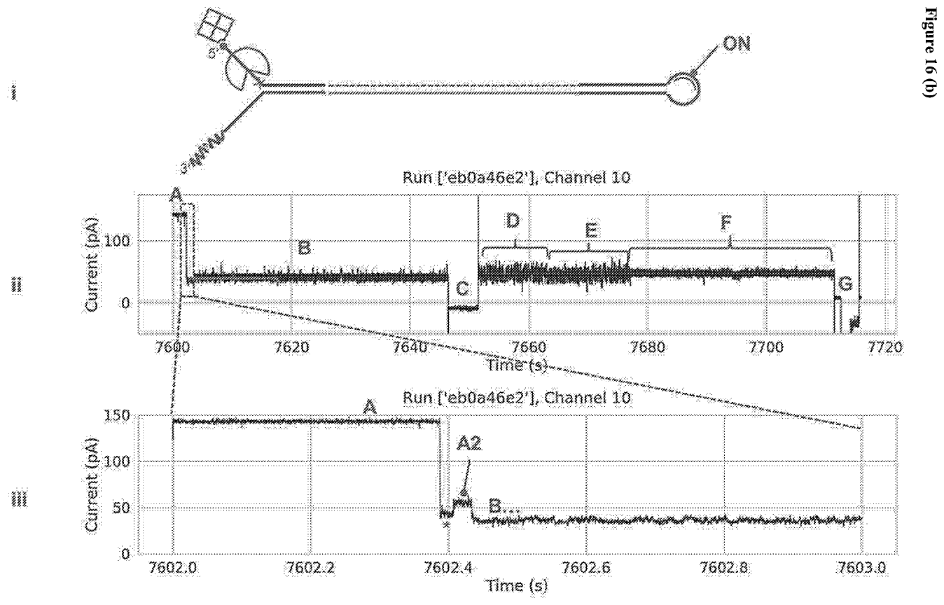

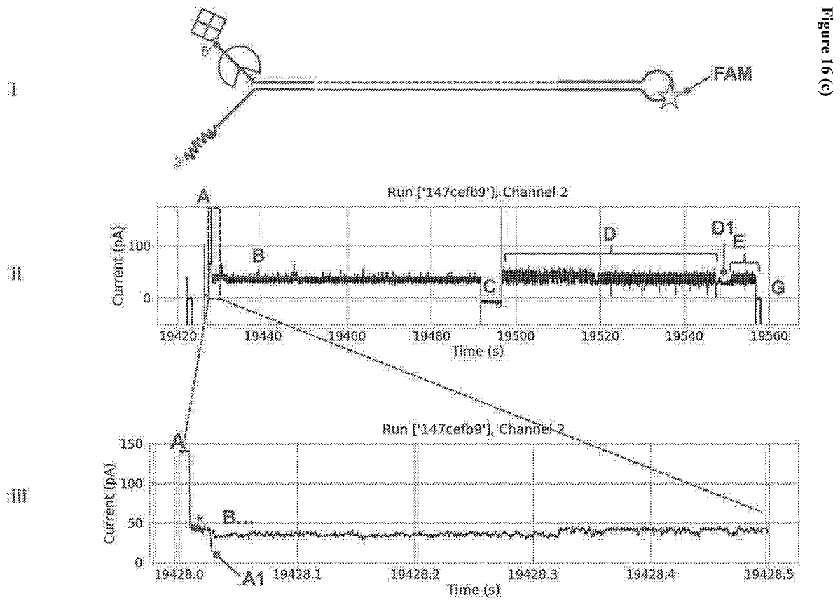

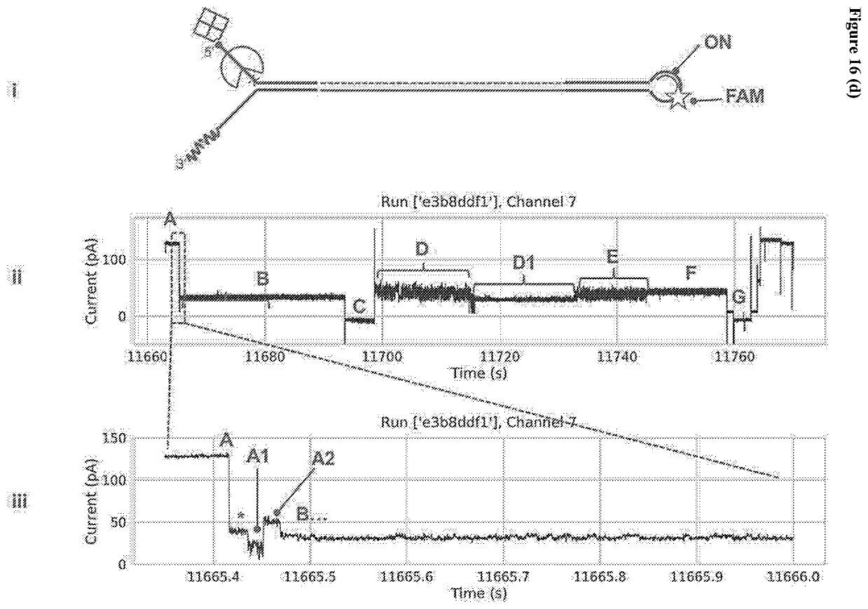

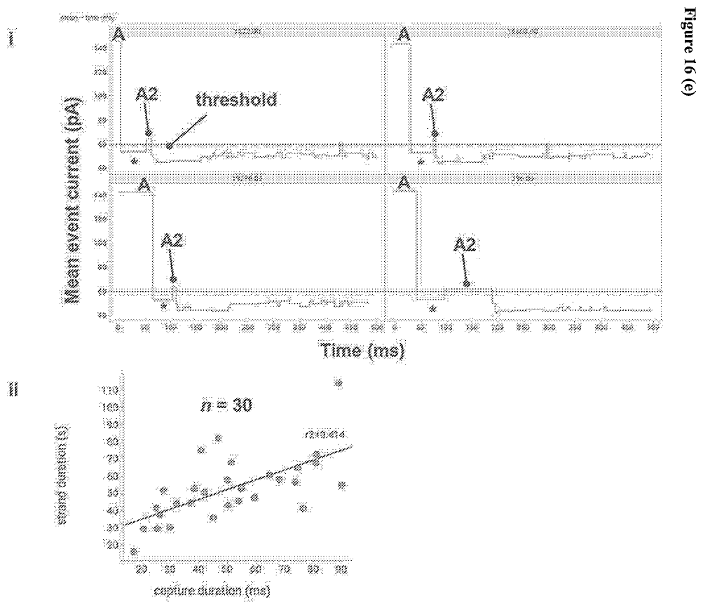

In some embodiments the target polynucleotide is double-stranded and comprises a first strand and a second strand; the target polynucleotide comprises a leader sequence, wherein the leader sequence is located at a first end of the polynucleotide and is comprised in the first strand or is comprised in an adapter attached to the first strand; the first strand and the second strand are attached together by a hairpin adapter at the second end of the first strand; and the motor protein is stalled at the hairpin adapter. In such embodiments, the leader sequence is typically captured by the nanopore and the first strand of the double-stranded polynucleotide translocates through the nanopore until the stalled motor protein is reached. Once destalled, the motor protein controls the movement of the first strand of the double-stranded polynucleotide out of the pore. This setup is illustrated schematically in FIG. 4.

In some embodiments the first strand and the second strand are attached together by a hairpin adapter attached to (i) the second end of the first strand and (ii) a first end of the second strand; and the motor protein is stalled at a second end of the second strand or is stalled on an adapter at the second end of the second strand of the double-stranded polynucleotide. In some embodiments the hairpin adapter is attached at its 5′ end to the 3′ end of the first strand and is attached at its 3′ end to the 5′ end of the second strand of the target double-stranded polynucleotide; and the motor protein is stalled at the 3′ end of the second strand. In some embodiments the hairpin adapter is attached at its 3′ end to the 5′ end of the first strand and is attached at its 5′ end to the 3′ end of the second strand of the target double-stranded polynucleotide, and the motor protein is stalled at the 5′ end of the second strand. Accordingly, the hairpin adapter connects the first strand to the second strand.

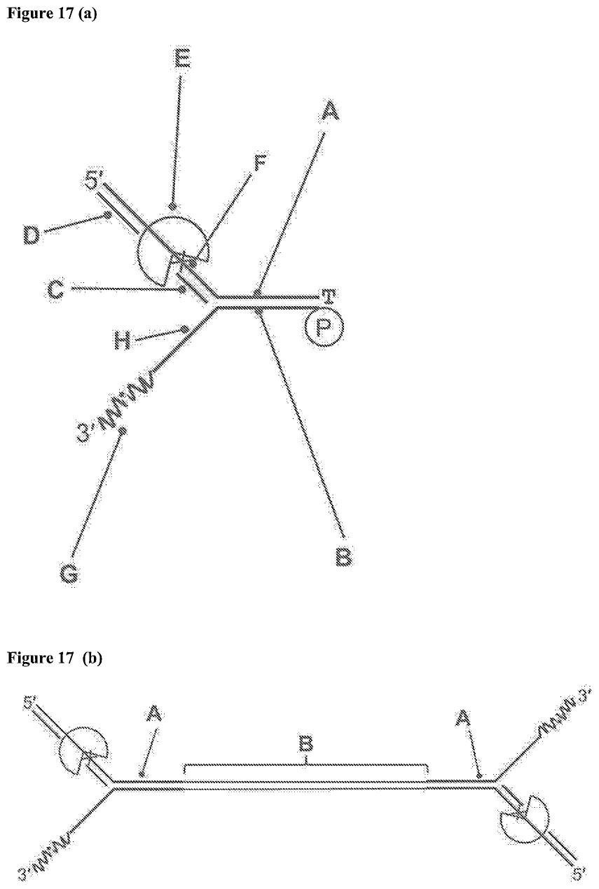

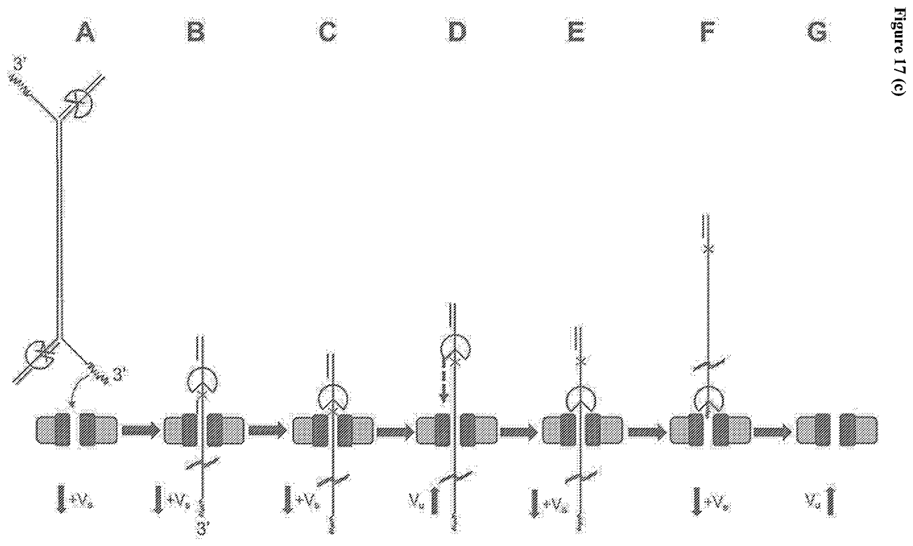

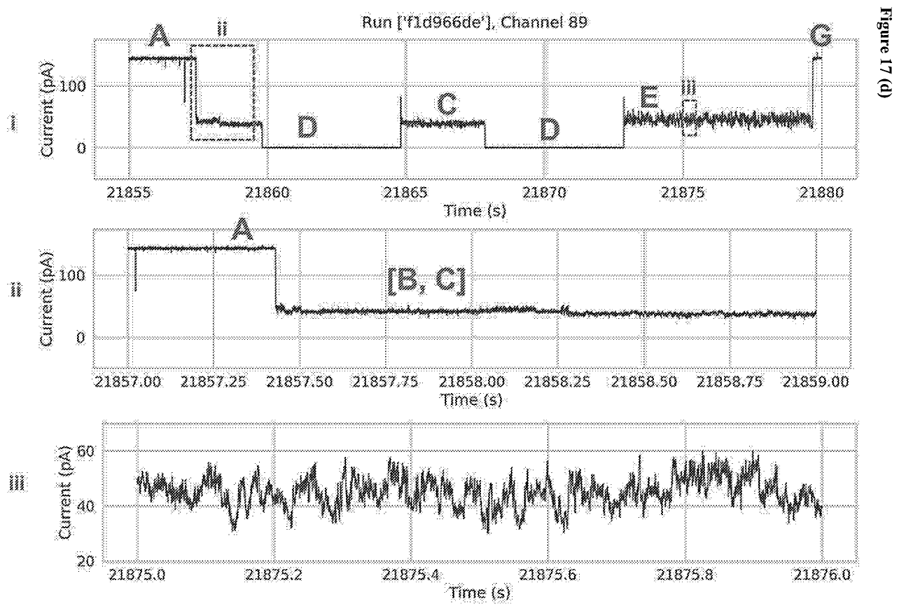

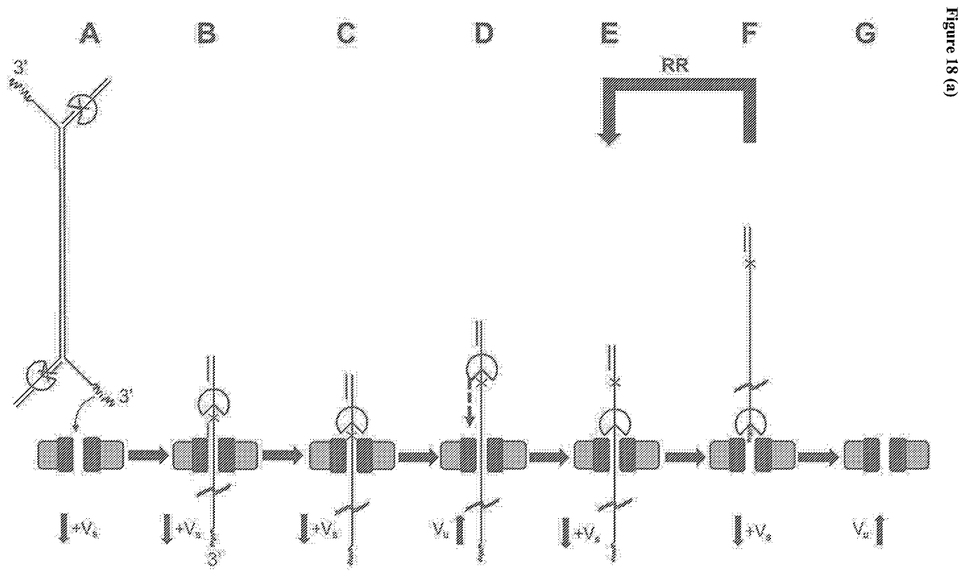

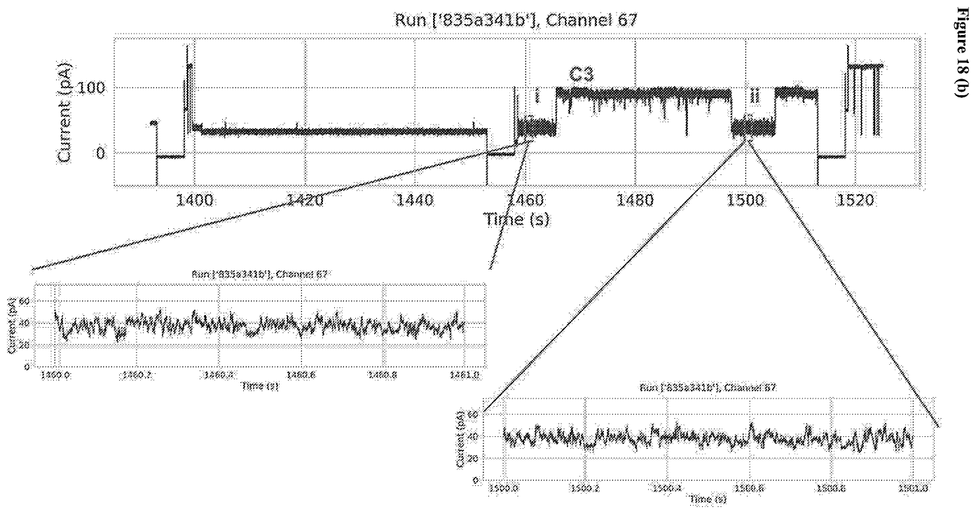

In some embodiments the target polynucleotide is double-stranded and comprises a first strand and a second strand; the target polynucleotide comprises a leader sequence, wherein the leader sequence is located at a first end of the polynucleotide and is comprised in the first strand or is comprised in an adapter attached to the first strand; the first strand and the second strand are attached together by a hairpin adapter attached to (i) the second end of the first strand and (ii) a first end of the second strand; and the motor protein is stalled at a second end of the second strand or is stalled on an adapter at the second end of the second strand of the double-stranded polynucleotide. In such embodiments, the leader sequence is typically captured by the nanopore and the first strand of the double-stranded polynucleotide, the hairpin adapter and the second strand of the double-stranded polynucleotide translocate through the nanopore until the stalled motor protein is reached. Once destalled, the motor protein controls the movement of the second strand, and optionally also the hairpin adapter and further optionally the first strand of the double-stranded polynucleotide out of the pore. This setup is illustrated schematically in FIG. 5.

It will be apparent that the motor protein may not be stalled at the terminus of the polynucleotide but may be stalled part way along the polynucleotide. As used herein, the motor protein is in such embodiments stalled at the end of the portion of the polynucleotide to be characterised in the methods provided herein. Those skilled in the art will appreciate that in the methods provided herein, the portion of the polynucleotide that is characterised can be determined by the positioning of the motor protein on the polynucleotide, and this is a parameter that can be controlled by the user of the method.

In embodiments of the disclosed methods which comprise re-reading the target polynucleotide (e.g. in methods which comprise taking one or more measurements characteristic of the target polynucleotide as the motor protein controls the movement of the target polynucleotide in a first direction with respect to the detector; unbinding the target polynucleotide from the polynucleotide binding site of the motor protein, such that the target polynucleotide moves in a second direction with respect to the detector; and re-binding the target polynucleotide to the polynucleotide binding site of the motor protein; and taking one or more measurements characteristic of the target polynucleotide as the motor protein controls the movement of the target polynucleotide in the first direction with respect to the detector), a leader sequence may be configured or designed in order to promote the unbinding of the target polynucleotide from the polynucleotide binding site of the motor protein when the motor protein is in the vicinity of the leader sequence, e.g. when the motor protein contacts the leader sequence.

In such embodiments, the motor protein typically has a lower affinity for the leader sequence than for the target polynucleotide, i.e. than for the portion of the target polynucleotide to be characterised. In some embodiments the leader has a different structure to the target polynucleotide. In some embodiments the leader comprises a different type of nucleotide to the target polynucleotide.

For example, in some embodiments the target polynucleotide comprises deoxyribonucleotides (DNA). In such embodiments, the leader may comprises one or more nucleotides lacking both nucleobase and sugar moieties (e.g. a spacer moiety). Suitable spacer moieties are described in more detail herein, and include C2 spacers, C3 spacers, C6 spacers, iSp9 spacers, iSp18 spacers etc. Alternatively or additionally, the leader may comprise ribonucleotides (RNA), peptide nucleotides (PNA), glycerol nucleotides (GNA), threose nucleotides (TNA), locked nucleotides (LNA), bridged nucleotides (BNA), or abasic nucleotides. In some embodiments the leader may comprise one or more nucleotides having a modified phosphate linkage (e.g. comprising a methylphosphonate or phosphothioate linkage).

In some other embodiments the target polynucleotide comprises ribonucleotides (RNA). In such embodiments, the leader may comprises one or more spacers as defined above, deoxyribonucleotides (DNA), peptide nucleotides (PNA), glycerol nucleotides (GNA), threose nucleotides (TNA), locked nucleotides (LNA), bridged nucleotides (BNA), abasic nucleotides or nucleotides comprising a modified phosphate linkage.

Typically, the target polynucleotide comprises deoxyribonucleotides (DNA) and the leader comprises one or more spacer moieties (e.g. C3 spacers) and/or one or more ribonucleotides.

The leader may comprise just one type of polynucleotide that is different to the target polynucleotide. For example, when the target polynucleotide is DNA the leader may comprise spacer moieties or RNA. The leader may comprise more than one type of polynucleotide that is different to the target polynucleotide. For example, when the target polynucleotide is DNA the leader may comprise spacer moieties and RNA. The leader may comprise portions which are of the same type of polynucleotide as the target polynucleotide. For example, when the target polynucleotide is DNA, the leader may comprise in addition to spacer polynucleotides or RNA, portions of DNA. Such portions may be referred to as “traps”; i.e. a leader based on spacer (e.g. C3 spacer) and/or RNA (e.g. 2′-methoxy uridine) polynucleotides may comprise one or more DNA traps. A trap typically comprises from 1 to 10 nucleotides, such as from 1 to 6 nucleotides e.g. 1, 2, 3, 4 or 5 nucleotides such as from 1 to 3 nucleotides. When the target polynucleotide is DNA, the leader may therefore comprise one or more RNA (e.g. 2′-methoxyuridine) and/or spacer (e.g. C3 spacer) moieties and one or more DNA (e.g. thymidine) traps of from 1 to 10 nucleotides in length.

Those skilled in the art will also appreciate that when the leader comprises a polynucleotide strand, the sequence of the leader is typically not determinative and can be controlled or chosen according to the motor protein and other experimental conditions such as any polynucleotide to be characterised. Exemplary sequences are provided solely by way of illustration in the examples, such as in example 10. For example, the leader may comprise a sequence such as one or more of SEQ ID NOs: 70, 71 or 72, or a polynucleotide sequence having at least 20%, such as at least 30%, e.g. at least 40% such as at least 50%, e.g. at least 60% such as at least 70%, e.g. at least 80%, for example at least 90% e.g. at least 95% sequence similarity or identity to one or more of SEQ ID NOs: 70, 71 or 72. The sequence of the leader can typically be altered without negatively affecting the efficacy of the methods provided herein.

### Stalling Motor Proteins

As explained above, the methods provided herein comprise characterisation of a target polynucleotide having a motor protein stalled thereon at a stalling moiety.

Any suitable stalling moiety can be used in the methods provided herein. In some embodiments the stalling moiety comprises a stalling site as described herein. In some embodiments the stalling site comprises one or more stalling units.

Any suitable stalling units can be used. A stalling unit typically provides an energy barrier which impedes movement of the motor protein. For example, a stalling unit may stall a motor protein by reducing the traction of the motor protein on the polynucleotide. This may be achieved for instance by using an abasic “spacer” i.e. a stalling unit in which the bases are removed from one or more nucleotides. A stalling unit may physically block movement of a motor protein, for instance by introducing a bulky chemical group to physically impede the movement of the protein.

In some embodiments, a stalling unit may comprise a linear molecule, such as a polymer. Typically, such a stalling unit has a different structure from the target polynucleotide. For instance, if the target polynucleotide is DNA, the or each stalling unit typically does not comprise DNA. In particular, if the target polynucleotide is deoxyribonucleic acid (DNA) or ribonucleic acid (RNA), the or each stalling unit preferably comprises peptide nucleic acid (PNA), glycerol nucleic acid (GNA), threose nucleic acid (TNA), locked nucleic acid (LNA), bridged nucleic acid (BNA) or a synthetic polymer with nucleotide side chains. In some embodiments, a stalling unit may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A stalling site may comprise any combination of these groups. Many of these groups are commercially available from IDT® (Integrated DNA Technologies®). For example, C3, iSp9 and iSp18 spacers are all available from IDT®. A stalling site may comprise any number of the above groups as stalling units. For instance, a stalling site may comprise from 1 to about 12 or more (e.g. from about 1 to about 8, for instance from 1 to about 6 such as from 1 to about 4) of such stalling units.

In some embodiments, a stalling unit may comprise one or more chemical groups which cause the a motor protein to stall. In some embodiments, suitable chemical groups are one or more pendant chemical groups. The one or more chemical groups may be attached to one or more nucleobases in the polynucleotide. The one or more chemical groups may be attached to the backbone of the polynucleotide. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

In some embodiments, a stalling unit may comprise a polymer. In some embodiments the stalling unit may comprise a polymer which is a polypeptide or a polyethylene glycol (PEG).

In some embodiments, a stalling unit may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic residues can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more stalling units do not comprise any abasic nucleotides.

Suitable stalling units can be designed or selected depending on the nature of the polynucleotide/polynucleotide adapter, the motor protein and the conditions under which the method is to be carried out. For example, many polynucleotide processing proteins process DNA in vivo and such proteins may typically be stalled using anything that is not DNA.

In some embodiments of the provided methods the motor protein is thus stalled at a stalling site comprising one or more stalling units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
spacer units selected from nitroindoles, inosines, acridines, 2-aminopurines, 2-6-diaminopurines, 5-bromo-deoxyuridines, inverted thymidines (inverted dTs), inverted dideoxy-thymidines (ddTs), dideoxy-cytidines (ddCs), 5-methylcytidines, 5-hydroxymethylcytidines, 2′-O-Methyl RNA bases, Iso-deoxycytidines (Iso-dCs), Iso-deoxyguanosines (Iso-dGs), C3 (OC₃H₆OPO₃) groups, photo-cleavable (PC) [OC₃H₆—C(O)NHCH₂—C₆H₃NO₂—CH(CH₃)OPO₃] groups, hexandiol groups, spacer 9 (iSp9) [(OCH₂CH₂)₃OPO₃] groups, spacer 18 (iSp18) [(OCH₂CH₂)₆OPO₃] groups; and thiol connections; and
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups.

Stalling moieties as described herein can also be used to configure a leader to be suitable for use in the disclosed re-reading methods. As explained above, in some embodiments of such methods a leader sequence as described herein is configured or designed in order to promote the unbinding of the target polynucleotide from the polynucleotide binding site of the motor protein when the motor protein is in the vicinity of the leader sequence, e.g. when the motor protein contacts the leader sequence. In some embodiments the leader sequence may comprise any of the spacer moieties described above.

### Destalling Motor Proteins

In some embodiments, methods provided herein comprise contacting the stalling moiety with the detector (e.g. the nanopore) thereby destalling the motor protein. Once destalled, the motor protein can control the movement of the polynucleotide out of the detector (e.g. out of the nanopore) as described in more detail herein.

In its simplest form, contacting the stalling moiety with the detector e.g. the nanopore may destall the motor protein from the stalling moiety. However, in some embodiments the method comprises actively destalling the motor protein as described herein.

In some embodiments, destalling the motor protein comprises applying a destalling force to the polynucleotide, wherein the destalling force is lower in magnitude and/or of opposite direction to a read force, wherein the read force is the force applied whilst the motor protein controls the movement of the target polynucleotide and the measurements to determine one or more characteristics of the polynucleotide are taken.

For example, the read force may typically be provided as a voltage potential of from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. The destalling force is typically lower in magnitude than the read force. For example, the destalling force may be from about −100 mV to +100 mV, such as from about −50 mV to about +50 mV, e.g. from about −25 mV to about +25 mV.

For example, in some embodiments the read force is a voltage potential of from +50 mV to +300 mV, more preferably in the range of +100 mV to +200 mV such as from +120 mV to +150 mV, and the destalling force is a voltage potential of from −50 to +50 mV such as from −40 mV to +40 mV, e.g. from −20 mV to +20 mV such as 0 mV.

In some embodiments the destalling force is opposite in direction to the read force. For example in some embodiments the read force is applied as a positive voltage potential and the destalling force is applied as a negative voltage potential. In other embodiments the read force is applied as a negative voltage potential and the destalling force is applied as a positive potential. When the destalling force is of opposite direction to the read force it may be of equal magnitude to the read force or may be of lower magnitude to the read force.

In some embodiments the destalling force is applied at zero potential. For example in some embodiments the read force is applied as a positive voltage potential and the destalling force is applied at zero applied potential. In other embodiments the read force is applied as a negative voltage potential and the destalling force is applied at zero applied potential.

In some embodiments the destalling force is applied for a time sufficient for the motor protein to destall from the stalling moiety. In some embodiments the destalling force is applied for between 1 ms to about 10 s, such as from about 10 ms to about 1 s, e.g. from about 100 ms to about 700 ms such as from about 300 ms to about 500 ms.

In some embodiments destalling the motor protein comprises altering the applied force one or more times between the destalling force and the read force. In some embodiments altering the applied force in this manner comprises stepping or ramping the applied potential between the destalling force and the read force. When ramped, any suitable waveform can be used, e.g. the ramp may be a linear ramp, an exponential ramp or a sigmoidal ramp.

In some embodiments the applied force is stepped between a single destalling force and the read force. In some embodiments the applied force is stepped between a series of different destalling forces and the read force. In some embodiments the applied force is stepped between a series of increasing destalling forces and the read force. The destalling forces at each step may be any suitable destalling force, e.g. any of the destalling forces described herein; and at each step may be applied for any suitable time duration e.g. any time duration as described herein.

In some embodiments the destalling force is the same as the read force. This is also referred to as destalling in a “free running” setup.

In some embodiments the motor protein is stalled at a stalling site comprising one or more stalling units and one or more pausing moieties; and wherein contacting the one or more pausing moieties with the nanopore retards the movement of the polynucleotide through the nanopore thereby causing the motor protein to destall from the one or more stalling units. Such embodiments are suitable for use in a free running setup.

In some embodiments the pausing moiety provides an energy barrier which impedes movement of the polynucleotide through the nanopore. For example, a pausing moiety may impede movement of the polynucleotide through the nanopore by providing a physical block that needs to be removed before the polynucleotide can pass through the nanopore.

Without being bound by theory, the inventors believe that the pausing moiety retards the movement of the polynucleotide through the nanopore for sufficient time for the motor protein to overcome the stalling unit(s) and destall.

In some embodiments the pausing moiety comprises one or more pausing units comprising a polynucleotide secondary structure, preferably a hairpin or G-quadruplex (TBA). Such secondary structures prevent the free passage of the polynucleotide through the nanopore. Contacting the pausing moiety with the nanopore causes the secondary structure to dissociate (e.g. to unwind). The time taken for the secondary structure to dissociate allows the motor protein to destall from the stalling unit(s).

In some embodiments the pausing moiety comprises one or more pausing units comprising a hybridised oligonucleotide. The oligonucleotide may hybridise to the target polynucleotide and prevent movement of the target polynucleotide through the nanopore. Contacting the pausing moiety with the nanopore causes the dissociation of the hybridised oligonucleotide from the target polynucleotide. The time taken for the hybridised oligonucleotide to dissociate from the target polynucleotide allows the motor protein to destall from the stalling unit(s).

In some embodiments the pausing moiety comprises one or more pausing units comprising a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides. The nucleic acid analog may be provided in line with the target polynucleotide or may be hybridised or otherwise attached to the target polynucleotide. When the nucleic acid analog is provided in line with the target polynucleotide, contacting the pausing moiety with the nanopore causes the nucleic acid analog to pass through the nanopore. The time taken for the nucleic acid analog to pass through the pore allows the motor protein to destall from the stalling unit(s). When the nucleic acid analog is hybridised to the target polynucleotide, contacting the pausing moiety with the nanopore typically causes the nucleic acid analog to dissociate from the target polynucleotide such that the target polynucleotide can pass through the nanopore. The time taken for the nucleic acid analog to dissociate from the polynucleotide allows the motor protein to destall from the stalling unit(s).

In some embodiments the pausing moiety comprises one or more pausing units comprising a chemical group such as fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups. The chemical groups may be attached to the target polynucleotide and prevent the movement of the target polynucleotide through the nanopore. In some embodiments contacting the pausing moiety with the nanopore causes the chemical group to be removed from the target polynucleotide. In some embodiments contacting the pausing moiety with the nanopore causes the chemical group to be passed through the nanopore. The time taken for the chemical group to be removed from the target polynucleotide and/or to pass through the nanopore allows the motor protein to destall from the stalling unit(s).

In some embodiments the pausing moiety comprises one or more pausing units comprising a polynucleotide binding protein. Suitable polynucleotide binding proteins are described in more detail herein. The polynucleotide binding protein may be bound to the polynucleotide and prevent movement of the polynucleotide through the nanopore. Contacting the pausing moiety with the nanopore retards the movement of the polynucleotide through the nanopore, e.g. as the polynucleotide binding protein moves to contact the motor protein. The time taken to do allows the motor protein to destall from the stalling unit(s).

Without being bound by theory the inventors also believe that the pausing moiety often determines the conformation of the polynucleotide at the stalling unit(s). This is particularly the case when the stalling moiety comprises a linear group such as one or more spacer 18 (iSp18) [(OCH₂CH₂)₆OPO₃]. Without being bound by theory, it is believed that when such stalling unit(s) are in contact with a nanopore any applied force across the pore (e.g. an applied voltage field) can cause the stalling moiety to stretch out in an approximately linear manner. In this conformation, the motor protein is typically incapable of passing over the stalling moiety to destall. However, when the target polynucleotide is paused at a pausing moiety the environment at the stalling unit is believed to be similar to that in solution and the stalling unit may adopt a more compact pseudo-random coil configuration. In this configuration it may be easier for the motor protein to overcome the stalling unit and destall.

Accordingly, in some embodiments the motor protein is stalled at a stalling site comprising one or more stalling units and one or more pausing units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups; and
a polynucleotide binding protein;
and contacting the one or more pausing moieties with the nanopore retards the movement of the polynucleotide through the nanopore thereby causing the motor protein to destall from the one or more stalling units.

### Motor Protein

As those skilled in the art will appreciate, any suitable motor protein can be used in the methods and products provided herein.

The motor protein may be any protein that is capable of binding to a polynucleotide and controlling its movement with respect to a detector, e.g. a nanopore, e.g. through the pore.

In more detail, motor proteins such as helicases can typically control the movement of DNA in at least two active modes of operation (when is provided with all the necessary components to facilitate movement e.g. ATP and Mg²⁺) and one inactive mode of operation (when not provided with the necessary components to facilitate movement; or when the motor protein is modified in order to prevent the active mode).

When provided with all the necessary components to facilitate movement, a motor protein may move along a polynucleotide such as DNA in either a 5′-3′ direction or a 3′-5′ direction. Many motor proteins process polynucleotides such as DNA in a 5′-3′ direction. Motor proteins which control the movement of polynucleotides in this manner are typically suitable for use in the methods provided herein.

However, when a motor protein is not provided with the necessary components to facilitate movement, or is modified in order to prevent it from actively controlling the movement of the polynucleotide with respect to the nanopore, it can still passively control the movement of the polynucleotide with respect to the nanopore. For example, the motor protein can bind to the polynucleotide and act as a brake slowing the movement of the polynucleotide when it is pulled into the pore by an applied field (e.g. by the first force in the methods provided herein). In the “inactive” mode it typically does not matter whether the DNA is captured either 3′ or 5′ down (i.e. moves through the nanopore in a 5′-3′ direction or in a 3′-5′ direction), as the applied force provides the impetus to move the polynucleotide through the nanopore. However, in such embodiments the motor protein may still control the movement of the polynucleotide with respect to the nanopore e.g. by acting as a brake. When in the inactive mode the movement control of a polynucleotide by a motor protein can be described in a number of ways including ratcheting, sliding and braking. Typically the methods provided herein do not comprise the use of a motor protein operating in the passive mode. However in embodiments of the methods provided herein which use a polynucleotide binding protein the polynucleotide binding protein may be a motor protein operating in the passive mode.

As explained above, some embodiments of the methods provided herein also comprise the use of a polynucleotide binding protein as a pausing moiety to impede the movement of the polynucleotide strand through the nanopore. In some embodiments a polynucleotide binding protein may be a motor protein as described herein. In other embodiments a polynucleotide binding protein may be a protein which binds to polynucleotides but which does not have polynucleotide processing capacity; i.e. in some embodiments it is not a motor protein.

A polynucleotide-handling enzyme is a polypeptide that is capable of interacting with a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. A motor protein as used herein may be, or may be derived from a polynucleotide handling enzyme. A polynucleotide binding protein may be, or may be derived from a polynucleotide-handling enzyme.

In one embodiment, the motor protein and/or polynucleotide binding protein is derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

Typically, the motor protein and/or polynucleotide binding protein is a helicase, a polymerase, an exonuclease, a topoisomerase, or a variant thereof.

In some embodiments, the motor protein and/or polynucleotide binding protein may be modified to prevent the motor protein disengaging from the polynucleotide. This is particularly useful in the methods disclosed herein which comprise re-reading the target polynucleotide. Thus, in some embodiments of such methods, the target polynucleotide does not disengage from the motor protein.

As used herein, the term “disengaging” refers to the dissociation of the motor protein from the target polynucleotide. Thus, a motor protein may be modified to prevent it from dissociating from the target polynucleotide, e.g. into the reaction medium. It is important to distinguish potential “disengagement” of a motor protein from “unbinding” of a motor protein from a target polynucleotide. As used herein, “unbinding” refers to the transient release of the target polynucleotide the active site of the motor protein (described in more detail herein) but does not imply disengagement. Thus, for example, a motor protein may be modified to prevent the motor protein from disengaging from a polynucleotide, but without preventing the motor protein from unbinding from the polynucleotide. When unbound, the motor protein remains engaged with the target polynucleotide. For example, the motor protein may remain engaged with the target polynucleotide (i.e. it may be prevented from disengaging from the target polynucleotide). because it is topologically closed around the target polynucleotide. The polynucleotide binding site may remain free to bind or unbind the target polynucleotide such that the motor protein may bind or unbind to the target polynucleotide, whilst the motor protein remains engaged with the target polynucleotide. When the motor protein is unbound from the target polynucleotide it may be able to move on (e.g., along) the target polynucleotide under an applied force and may be capable of re-binding to the target polynucleotide. When engaged on the target polynucleotide but unbound from the target polynucleotide, the motor protein is not capable of dissociating from the target polynucleotide.

The motor protein and/or polynucleotide binding protein can be adapted to prevent disengagement in any suitable way. For example, the motor protein and/or polynucleotide binding protein can be loaded on the polynucleotide and then modified in order to prevent it from disengaging from the polynucleotide. Alternatively, the motor protein and/or polynucleotide binding protein can be modified to prevent it from disengaging from the polynucleotide before it is loaded onto the polynucleotide. Modification of a motor protein and/or a polynucleotide binding protein in order to prevent it from disengaging from a polynucleotide can be achieved using methods known in the art, such as those discussed in WO 2014/013260, which is hereby incorporated by reference in its entirety, and with particular reference to passages describing the modification of motor proteins such as helicases in order to prevent them from disengaging with polynucleotide strands. For example, a motor protein and/or polynucleotide binding protein can be modified by treating with tetramethylazodicarboxamide (TMAD). Various other closing moieties are described in more detail herein.

For example, a motor protein and/or a polynucleotide binding protein may have a polynucleotide-unbinding opening; e.g. a cavity, cleft or void through which a polynucleotide strand may pass when the motor protein/polynucleotide binding protein disengages from the strand. In some embodiments, the polynucleotide-unbinding opening is the opening through which a polynucleotide may pass when the motor protein/polynucleotide binding protein disengages from the polynucleotide. In some embodiments, the polynucleotide-unbinding opening for a given motor protein/polynucleotide binding protein can be determined by reference to its structure, e.g. by reference to its X-ray crystal structure. The X-ray crystal structure may be obtained in the presence and/or the absence of a polynucleotide substrate. In some embodiments, the location of a polynucleotide-unbinding opening in a given motor protein/polynucleotide binding protein may be deduced or confirmed by molecular modelling using standard packages known in the art. In some embodiments, the polynucleotide-unbinding opening may be transiently produced by movement of one or more parts e.g. one or more domains of the motor protein.

The motor protein/polynucleotide binding protein may be modified by closing the polynucleotide-unbinding opening. The polynucleotide-unbinding opening may be closed with a closing moiety. Closing the polynucleotide-unbinding opening may therefore prevent the motor protein/polynucleotide binding protein from disengaging from the polynucleotide. For example, the motor protein and/or polynucleotide binding protein may be modified by covalently closing the polynucleotide-unbinding opening. However, as explained above closing the polynucleotide-unbinding opening does not necessarily prevent the target polynucleotide from unbinding from the polynucleotide binding site of the motor protein. In some embodiments, a preferred protein for addressing in this way is a helicase.

In some embodiments, especially in embodiments of the disclosed methods which comprise re-reading the target polynucleotide, the motor protein may be modified to prevent the target polynucleotide disengaging from the target polynucleotide. The motor protein may be modified in any suitable manner.

Without being bound by theory, the inventors believe that promoting unbinding and retarding re-binding may promote re-reading. Without being bound by theory, the inventors believe that this may be because each step that a motor protein takes on a target polynucleotide is associated with a probability of the motor protein unbinding from the polynucleotide. The likelihood of such unbinding may be identified with the so-called off-rate. Increasing the off-rate is believed to promote drop-back of the motor protein with respect to the polynucleotide strand. Similarly, and again without being bound by theory, the inventors believe that once unbound from a target polynucleotide, the distance that the motor protein may move along the target polynucleotide before re-binding is associated with the on-rate. Thus, re-reading may be promoted by increasing the off-rate and decreasing the on-rate of the motor protein with respect to the target polynucleotide. Tailoring the off- and on-rates of the motor protein for a given type of polynucleotide is within the abilities of those of skill in the art in view of the disclosure herein. Accordingly, the motor protein may be modified to promote unbinding of the target polynucleotide from the polynucleotide binding site of the motor protein and/or to retard re-binding of the target polynucleotide to the polynucleotide binding site of the motor protein. In some embodiments the motor protein is modified to both promote unbinding of the target polynucleotide from the polynucleotide binding site of the motor protein and to retard re-binding of the target polynucleotide to the polynucleotide binding site of the motor protein.

In some embodiments the motor protein may be modified with a closing moiety for (i) topologically closing the polynucleotide binding site of the motor protein around the target polynucleotide and (ii) promoting unbinding of the target polynucleotide from the polynucleotide binding site of the motor protein and/or retarding re-binding of the target polynucleotide to the polynucleotide binding site of the motor protein. The motor protein may be modified in any suitable manner to facilitate attachment of such a closing moiety.

In some embodiments a closing moiety may comprise a bifunctional cross-linking moiety. The closing moiety may comprise a bifunctional cross-linker. The bifunctional crosslinker may attach at two points on the motor protein and close the polynucleotide-unbinding opening of the motor protein thereby preventing disengagement of the polynucleotide from the motor protein whilst allowing unbinding of the polynucleotide from the polynucleotide-binding site of the motor protein.

The closing moiety may attach at any suitable positions on the motor protein. For example, the closing moiety may crosslink two amino acid residues of the motor protein. Typically, at least one amino acid crosslinked by the closing moiety is a cysteine or a non-natural amino acid. The cysteine or non-natural amino acid may be introduced into the motor protein by substitution or modification of a naturally occurring amino acid residue of the motor protein. Methods for introducing non-natural amino acids are well known in the art and include for example native chemical ligation with synthetic polypeptide strands comprising such non-natural amino acids. Methods for introducing cysteines into a motor protein are likewise within the capability of one of skill in the art, for example using techniques disclosed in references such as Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016).

In some embodiments the closing moiety has a length of from about 1 Å to about 100 Å. The length of the closing moiety may be calculated according to static bond lengths or more preferably using molecular dynamics simulations. The length may for example be from about 2 Å to about 80 Å, such as from about 5 Å to about 50 Å, e.g. from about 8 to about 30 Å such as from about 10 to about 25 Å or about 20 Å, e.g. about 1, 2, 3, 4, 5, 6, 7, 8, 9, 10, 11, 12, 13, 14, 15, 16, 17, 18, or 19 Å.

Without in any way being bound by theory, the inventors consider that in general longer closing moieties may increase the off-rate of the motor protein from the polynucleotide and thus promote re-reading.

In some embodiments the closing moiety comprises a bond. In some embodiments the closing moiety comprises a disulphide bond. A disulphide bond may be formed by treating the motor protein with any suitable reagent such as TMAD.

In some embodiments the closing moiety comprises a reagent which forms bonds between two click chemistry groups on the motor protein. Examples of click chemistry reagents are provided herein.

In some embodiments the closing moiety comprises a protein. For example, biotin groups may be present on the motor protein and the closing moiety may comprise streptavidin. Tags such as snoop-tag or spy-tag may be present on the motor protein and the closing moiety may comprise a protein such as snoop-catcher or spy-catcher, respectively.

In some embodiments the closing moiety comprises a structure of formula [A-B-C], wherein A and C are each independently reactive functional groups for reacting with amino acid residues in the motor protein and B is a linking moiety. In some embodiments the closing moiety comprises a link between thio groups e.g. thiol groups on cysteine residues. In some embodiments therefore A and C are cysteine-reactive functional groups. In some embodiments linking moiety B comprises a linear or branched, unsubstituted or substituted alkylene, alkenylene, alkynylene, arylene, heteroarylene, carbocyclylene or heterocyclylene moiety, which moiety is optionally interrupted by and/or terminated in one or more atoms or groups selected from O, N(R), S, C(O), C(O)NR, C(O)O, unsubstituted or substituted arylene, arylene-alkylene, heteroarylene, heteroarylene-alkylene, carbocyclylene, carbocyclylene-alkylene, heterocyclylene and heterocyclylene-alkylene; wherein R is selected from H, unsubstituted or substituted alkyl, and unsubstituted or substituted aryl. Typically R is H or methyl, more typically H.

Typically, an alkylene group is a C₁₋₂₀ alkylene group. Typically, an alkenylene group is a C₂₋₂₀ alkenylene group. Typically, an alkynylene group is a C₂₋₂₀ alkynylene group. Typically, an arylene group is a C₆₋₁₂ arylene group. Typically, a heteroarylene group is a 5- to 12-membered heteroarylene group. Typically, a carbocyclylene group is a C₅₋₁₂ carbocyclylene group. Typically, a heterocyclylene group is a 5- to 12-membered heterocyclylene group.

Typically, an alkylene, alkenylene, or alkynylene moiety may be uninterrupted or interrupted by or terminate in one or more atoms or groups selected from O, N(R), S, C(O), C(O)NR, and C(O)O and unsubstituted or substituted arylene. Usually, an alkylene, alkenylene, or alkynylene moiety may be uninterrupted or interrupted by or terminate in one or more atoms or groups selected from O and N(R) and unsubstituted or substituted arylene. More often, an alkylene, alkenylene, or alkynylene moiety may be uninterrupted or interrupted by or terminate in one or more O atoms.

For example, a linking moiety is often an unsubstituted or substituted C₁₋₁₀ alkylene, C₂₋₁₀ alkenylene, or C₂₋₁₀ alkynylene moiety which is uninterrupted or interrupted by or terminates in one or more O atoms.

In some embodiments, linking moiety B comprises an alkylene, oxyalkylene or polyoxyalkylene group and/or wherein A and C are each maleimide groups. The alkylene, oxyalkylene or polyoxyalkylene group may for example have a length of from about 5 Å to about 50 Å, e.g. from about 8 to about 30 Å such as from about 10 to about 25 Å.

For example, a linking moiety may comprise a PEG moiety such as (CH₂CH₂O)ₓ wherein x is from 1 to 10, e.g. from 1 to 5, e.g. 1, 2 or 3. Exemplary linking moieties are described in example 9 and include for example BMOE (1,2-bismaleimidoethane), BMOP (1,3-bismaleimidopropane), BMB (1,4-bismaleimidobutane), BM(PEG)₂ (1,8-bismaleimido-diethyleneglycol) and BM(PEG)₃ (1,11-bismaleimido-triethyleneglycol).

Motor proteins suitable for being closed using a closing moiety as described above are discussed in more detail herein. In some preferred embodiments the motor protein is a helicase, e.g. a Dda helicase as described herein.

In one embodiment, the motor protein and/or the polynucleotide binding protein is or is derived from an exonuclease. Suitable enzymes include, but are not limited to, exonuclease I from E. coli (SEQ ID NO: 1), exonuclease III enzyme from E. coli (SEQ ID NO: 2), RecJ from T. thermophilus (SEQ ID NO: 3) and bacteriophage lambda exonuclease (SEQ ID NO: 4), TatD exonuclease and variants thereof. Three subunits comprising the sequence shown in SEQ ID NO: 3 or a variant thereof interact to form a trimer exonuclease.

In one embodiment, the motor protein and/or the polynucleotide binding protein is a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase (SEQ ID NO: 5) or a variant thereof. Modified versions of Phi29 polymerase that may be used in the invention are disclosed in U.S. Pat. No. 5,576,204.

In one embodiment the motor protein and/or the polynucleotide binding protein is a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

In one embodiment, the motor protein and/or the polynucleotide binding protein is a helicase. Any suitable helicase can be used in accordance with the methods provided herein. For example, the or each enzyme used in accordance with the present disclosure may be independently selected from a Hel308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep, PcrA, Pif1 and TraI. These helicases typically work on single stranded DNA. Examples of helicases that can move along both strands of a double stranded DNA include FtfK and hexameric enzyme complexes, or multisubunit complexes such as RecBCD. In one embodiment the motor protein is a Dda (DNA-dependent ATPase) helicase.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

In one embodiment a helicase may comprise the sequence shown in SEQ ID NO: 6 (Trwc Cba) or a variant thereof, the sequence shown in SEQ ID NO: 7 (Hel308 Mbu) or a variant thereof or the sequence shown in SEQ ID NO: 8 (Dda) or a variant thereof. Variants may differ from the native sequences in any of the ways discussed herein. An example variant of SEQ ID NO: 8 comprises E94C/A360C. A further example variant of SEQ ID NO: 8 comprises E94C/A360C and then (ΔM1)G1G2 (i.e. deletion of M1 and then addition of G1 and G2).

Typically, a motor protein or polynucleotide binding protein may have a fuel binding site. The active unwinding of DNA may be coupled to fuel hydrolysis, e.g. in the motor protein.

Fuel is typically free nucleotides or free nucleotide analogues. The free nucleotides may be one or more of, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are usually selected from AMP, TMP, GMP, CMP, UMP, dAMP, dTMP, dGMP or dCMP. The free nucleotides are typically adenosine triphosphate (ATP).

A cofactor for the motor protein is a factor that allows the motor protein to function. The cofactor is preferably a divalent metal cation. The divalent metal cation is preferably Mg²⁺, Mn²⁺, Ca²⁺ or Co²⁺. The cofactor is most preferably Mg²⁺.

In some embodiments the polynucleotide binding protein is other than a motor protein as used herein. As used herein, the term polynucleotide binding protein and polynucleotide binding moiety can be used interchangeable.

For example, the polynucleotide binding protein or polynucleotide binding moiety may comprise one or more domains independently selected from helix-hairpin-helix (HhH) domains, eukaryotic single-stranded binding proteins (SSBs), bacterial SSBs, archaeal SSBs, viral SSBs, double-stranded binding proteins, sliding clamps, processivity factors, DNA binding loops, replication initiation proteins, telomere binding proteins, repressors, zinc fingers and proliferating cell nuclear antigens (PCNAs).

Helix-hairpin-helix (HhH) domains are polypeptide motifs that bind DNA in a sequence non-specific manner. Suitable domains include domain H (residues 696-751) and domain HI (residues 696-802) from Topoisomerase V from Methanopyrus kandleri (SEQ ID NO: 54). The polynucleotide binding moiety may be domains H-L of SEQ ID NO: 54 as shown in SEQ ID NO: 55 or a polynucleotide-binding variant thereof. The HhH domain may comprise the sequence shown in SEQ ID NO: 40 or 48 or 49 or a polynucleotide-binding variant thereof.

SSBs bind single stranded DNA with high affinity in a sequence non-specific manner. SSBs fall into the following lineage: Class; All beta proteins, Fold; OB-fold, Superfamily: Nucleic acid-binding proteins, Family; Single strand DNA-binding domain, SSB. The SSB may be from a eukaryote, such as from humans, mice, rats, fungi, protozoa or plants, from a prokaryote, such as bacteria and archaea, or from a virus. Eukaryotic SSBs are also known as replication protein A (RPAs). In most cases, they are hetero-trimers formed of different size units. Some of the larger units (e.g. RPA70 of Saccharomyces cerevisiae) are stable and bind ssDNA in monomeric form. Bacterial SSBs bind DNA as stable homo-tetramers (e.g. E. coli, Mycobacterium smegmatis and Helicobacter pylori) or homo-dimers (e.g. Deinococcus radiodurans and Thermotoga maritima). A few, such as the SSB encoded by the crenarchaeote Sulfolobus solfataricus, are homo-tetramers. Some SSBs from other species have been shown to be monomeric (Methanococcus jannaschii and Methanothermobacter thermoautotrophicum). Still other species of Archaea, including Archaeoglobus fulgidus and Methanococcoides burtonii, contain two open reading frames with sequence similarity to RPAs. Viral SSBs bind DNA as monomers.

The SSB is typically chosen or modified to have a carboxy-terminal (C-terminal) region which as no net negative charge or has a reduced net negative charge relative to the wild-type protein. Such SSBs typically do not block transmembrane pores. The C-terminal region of the SSB is typically about the last third, quarter, fifth or eighth of the SSB at the C-terminal end. The C-terminal region is typically from about the last 10 to about the last 60 amino acids of the C-terminal end of the SSB, e.g. from about the last 20 to last 40 such as last 30 amino acids of the C-terminal end of the SSB.

Examples of SSBs comprising a C-terminal region which does not have a net negative charge include the human mitochondrial SSB (HsmtSSB; SEQ ID NO: 50, the human replication protein A 70 kDa subunit, the human replication protein A 14 kDa subunit, the telomere end binding protein alpha subunit from Oxytricha nova, the core domain of telomere end binding protein beta subunit from Oxytricha nova, the protection of telomeres protein 1 (Pot1) from Schizosaccharomyces pombe, the human Pot1, the OB-fold domains of BRCA2 from mouse or rat, the p5 protein from phi29 (SEQ ID NO: 51); and polynucleotide-binding variants thereof. Examples of SSBs which can be modified in their C-terminal region to decrease the net negative charge include the SSB of E. coli (EcoSSB; SEQ ID NO: 52, the SSB of Mycobacterium tuberculosis, the SSB of Deinococcus radiodurans, the SSB of Thermus thermophiles, the SSB from Sulfolobus solfataricus, the human replication protein A 32 kDa subunit (RPA32) fragment, the CDC13 SSB from Saccharomyces cerevisiae, the Primosomal replication protein N (PriB) from E. coli, the PriB from Arabidopsis thaliana, the hypothetical protein At4g28440, the SSB from T4 (gp32; SEQ ID NO: 53), the SSB from RB69 (gp32; SEQ ID NO: 41), the SSB from T7 (gp2.5; SEQ ID NO: 42), and polynucleotide-binding variants thereof. Suitable modifications for decreasing the net negative charge are disclosed in WO 2014/013259.

Double-stranded binding proteins bind double stranded DNA with high affinity. Suitable double-stranded binding proteins include, but are not limited to Mutator S (MutS; NCBI Reference Sequence: NP_417213.1; SEQ ID NO: 56), Sso7d (Sulfolobus solfataricus P2; NCBI Reference Sequence: NP_343889.1; SEQ ID NO: 57; Nucleic Acids Research, 2004, Vol 32, No. 3, 1197-1207), Sso10b1 (NCBI Reference Sequence: NP_342446.1; SEQ ID NO: 58), Sso10b2 (NCBI Reference Sequence: NP_342448.1; SEQ ID NO: 59), Tryptophan repressor (Trp repressor; NCBI Reference Sequence: NP_291006.1; SEQ ID NO: 60), Lambda repressor (NCBI Reference Sequence: NP_040628.1; SEQ ID NO: 61), Cren7 (NCBI Reference Sequence: NP_342459.1; SEQ ID NO: 62), major histone classes H1/H5, H2A, H2B, H3 and H4 (NCBI Reference Sequence: NP_066403.2, SEQ ID NO: 63), dsbA (NCBI Reference Sequence: NP_049858.1; SEQ ID NO: 64), Rad51 (NCBI Reference Sequence: NP_002866.2; SEQ ID NO: 65), sliding clamps and Topoisomerase V Mka (SEQ ID NO: 54) or a polynucleotide-binding variant of any of these proteins.

Other polynucleotide binding proteins include sliding clamps. Sliding clamps are typically multimeric proteins (homo-dimers or homo-trimers) that encircle dsDNA. Sliding clamps typically require accessory proteins (clamp loaders) to assemble them around the DNA helix in an ATP-dependent process. They also do not contact DNA directly, acting as a topological tether. Related to DNA sliding clamps are processivity factors, which are viral proteins that anchor their cognate polymerases to DNA, leading to a dramatic increase in the length of the fragments generated. They can be monomeric (as is the case for UL42 from Herpes simplex virus 1) or multimeric (UL44 from Cytomegalovirus is a dimer). UL42 typically comprises the sequence shown in SEQ ID NO: 43 or SEQ ID NO: 47 or a polynucleotide-binding variant thereof.

Another polynucleotide binding protein is the thioredoxin binding domain (TBD) of bacteriophage T7 DNA polymerase (residues 258-333). Binding of TBD to thioredoxin (e.g. from E. coli) causes the polypeptide to change conformation to one that binds DNA. Other polynucleotide binding proteins include the accessory proteins cisA from phage Φx174 and geneII protein from phage M13. These proteins have intrinsic DNA binding capabilities, some of them recognizing a specific DNA sequence. Other polynucleotide binding proteins include telomeric binding proteins.

Small DNA binding motifs (such as helix-turn-helix) recognize specific DNA sequences. In the case of the bacteriophage 434 repressor, a 62 residue fragment was engineered and shown to retain DNA binding abilities and specificity. Zinc fingers consist of around 30 amino-acids that bind DNA in a specific manner. Typically each zinc finger recognizes only three DNA bases, but multiple fingers can be linked to obtain recognition of a longer sequence.

Proliferating cell nuclear antigens (PCNAs) form a very tight clamp which slides up and down the dsDNA or ssDNA. The PCNA from crenarchaeota is a hetero-trimer of SEQ ID NOs: 44, 45 and 46. A polynucleotide binding protein may thus be a trimer comprising the sequences shown in SEQ ID NOs: 44, 45 and 46 or polynucleotide-binding variants thereof. Another PCNA sliding clamp (NCBI Reference Sequence: ZP_06863050.1; SEQ ID NO: 66) forms a dimer. The polynucleotide binding protein may thus be a dimer comprising SEQ ID NO: 66 or a polynucleotide-binding variant thereof.

The polynucleotide binding motif may be selected from any of the following:

| Name | Class | Organism | Structure | Sequence | Functional form | MW (Da) |
|---|---|---|---|---|---|---|
| SSBEco | ssb | Escherichia coli | 1QVC, 1EYG | P0AGE0 | homo-tetramer | 18975 |
| SSBBhe | ssb | Bartonella henselae | 3LGJ, 3PGZ | Q6G302 | homo-tetramer | 16737 |
| SSBCbu | ssb | Coxiella burnetii | 3TQY | Q83EP4 | homo-tetramer | 17437 |
| SSBTma | ssb | Thermathoga maritima | 1Z9F | Q9WZ73 | homo-dimer | 16298 |
| SSBHpy | ssb | Helicobacter pylori | 2VW9 | O25841 | homo-tetramer | 20143 |
| SSBDra | ssb | Deinococcus radiodurans | 1SE8 | Q9RY51 | homo-dimer | 32722 |
| SSBTaq | ssb | Thermus aquaticus | 2FXQ | Q9KH06 | homo-dimer | 30026 |
| SSBMsm | ssb | Mycobacterium smegmatis | 3A5U, 1X3E | Q9AFI5 | homo-tetramer | 17401 |
| SSBSso | ssb/RPA | Sulfolobus solfataricus | 1O7I | Q97W73 | homo-tetramer | 16138 |
| SSBMHsmt | ssb | Homo sapiens | 3ULL | Q04837 | homo-tetramer | 17260 |
| SSBMle | ssb | Mycobacterium leprae | 3AFP | P46390 | homo-tetramer | 17701 |
| gp32T4 | ssb | Bacteriohage T4 | 1GPC | P03695 | monomer | 33506 |
| gp32RB69 | ssb | Bacteriophage RB69 | 2A1K | Q7Y265 | monomer | 33118 |
| gp2.5T7 | ssb | Bacteriohage T7 | 1JE5 | P03696 | monomer | 25694 |
| UL42 | processivity factor | Herpes virus 1 | 1DML | P10226 | monomer | 51159 |
| UL44 | processivity factor | Herpes virus 5 (cytomegalovirus) | 1YYP | P16790 | homo-dimer | 46233 |
| pf8 | processivity factor | KSHV | 3I2M | Q77ZG5 | homo-dimer | 42378 |
| RPAMja | RPA | Methanococcus jannaschii | 3DM3 | Q58559 | monomer | 73842 |
| RPAMma | RPA | Methanococcus maripaludis | 3E0E, 2K5V | Q6LYF9 | monomer | 71388 |
| RPAMth | RPA | Methanothermobacter | | | monomer | 120000 |
| | | thermoautotrophicus | | | | |
| RPA70Sce | RPA | Saccharomyces cerevisiae | 1YNX | P22336 | hetero-trimer | 70348 |
| RPAMbu1 | RPA | Methanococcoides burtonii | | Q12V72 | | 41227 |
| RPAMbu2 | RPA | Methanococcoides burtonii | | Q12W96 | | 47082 |
| RPA70Hsa | RPA | Homo sapiens | 1JMC | P27694 | hetero-trimer | 68138 |
| RPA14Hsa | RPA | Homo sapiens | 3KDF | P35244 | hetero-trimer | 13569 |
| gp45T4 | sliding clamp | Bacteriophage T4 | 1CZD | P04525 | homo-trimer | 24858 |
| BetaEco | sliding clamp | E. coli | 3BEP | P0A988 | homo-dimer | 40587 |
| PCNASce | sliding clamp | Saccharomyces cerevisiae | 1PLQ, 3K4X | P15873 | homo-dimer | 28916 |
| PCNATko | sliding clamp | Thermococcus kodakaraensis | 3LX1 | Q5JF32 | homo-dimer | 28239 |
| PCNAHvo | sliding clamp | Haloferax volcanii | 3IFV | D0VWY8 | homo-dimer | 26672 |
| PCNAPfu | sliding clamp | Pyrococcus furiosus | 1GE8 | O73947 | homo-dimer | 28005 |
| PCNAMbu | sliding clamp | Methanococcoides burtonii | | Q12U18 | homo-dimer | 27121 |
| BetaMtu | sliding clamp | Mycobacterium tuberculosis | 3P16 | Q50790 | homo-dimer | 42113 |
| BetaTma | sliding clamp | Thermotoga maritima | 1VPK | Q9WYA0 | homo-dimer | 40948 |
| BetaSpy | sliding clamp | Streptococcus pyrogenes | 2AVT | Q9EVR1 | homo-dimer | 41867 |
| gp45RB69 | sliding clamp | Bacteriophage RB69 | 1B77 | O80164 | homo-trimer | 25111 |
| p55Hsa | DNA binding protein | Homo sapiens (mitochondrial) | 2G4C, 3IKL, 3IKM | Q9UHN | monomer | 54911 |
| p55Dme | DNA binding protein | Drosophylla melanogaster | | Q9VJV8 | monomer | 41027 |
| p55Xla | DNA binding protein | Xenopus laevis | | Q9W6G7 | monomer | 52283 |
| RepDSau | replication initiation protein | Staphylococcus aureus | | P08115 | homo-dimer | 37874 |
| G2P | replication initiation protein | Enterobacteria phage 1 | | P69546 | monomer | 46168 |
| MutLEco | mismatch repair protein | Escherichia coli | 1BKN, 1B62, 1B63 | P23367 | homo-dimer | 67924 |
| KuMtu | DNA repair protein | Mycobacterium tuberculosis | | O05866 | homo-dimer | 30904 |
| OnTEBP | telomere binding protein | Oxytricha nova-Alpha | 1OTC | P29549 | hetero-dimer | 56082 |
| | | Oxytricha nova-Beta | | P16458 | | 41446 |
| EcrTEBP | telomere binding protein | Euplotes crassus | | Q06183 | monomer | 53360 |
| TteTEBP | telomere binding protein | Tetrachymena termophila Alpha | | Q23FB9 | hetero-dimer | 53073 |
| | | Tetrachymena termophila Beta | | Q23FH0 | | 54757 |
| pot1Spo | telomere binding proteins | Schizosaccharomyces pombe | | O13988 | monomer | 64111 |
| Cdc13pSce | telomere binding proteins | Saccharomyces cerevisiae | | C7GSV7 | monomer | 104936 |
| C1 | repressor | Bacteriophage 434 | | P16117 | homo-dimer | 10426 |
| LexA | repressor | Escherichia coli | 1LEB | P0A7C2 | homo-dimer | 22358 |

### Polynucleotide

The methods of the invention involve characterising a target polynucleotide as it moves with respect to a detector such as a nanopore.

A polynucleotide, such as a nucleic acid, is a macromolecule comprising two or more nucleotides. A polynucleotide can be single-stranded or double-stranded. A double-stranded polynucleotide is made of two single stranded polynucleotides hybridised together. The target polynucleotide can be a single-stranded polynucleotide or a double-stranded polynucleotide.

A polynucleotide may comprise any combination of any nucleotides. The nucleotides can be naturally occurring or artificial.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or ribonucleic acid (RNA). The polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA), bridged nucleic acid (BNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety.

The polynucleotide is preferably DNA, RNA or a DNA or RNA hybrid, most preferably DNA. A DNA/RNA hybrid may comprise DNA and RNA on the same strand. Preferably, the DNA/RNA hybrid comprises one DNA strand hybridized to a RNA strand.

The backbone of the polynucleotide can be altered to reduce the possibility of strand scission. For example, DNA is known to be more stable than RNA under many conditions. The backbone of the polynucleotide strand can be modified to avoid damage caused by e.g. harsh chemicals such as free radicals.

DNA or RNA that contains unnatural or modified bases can be produced by amplifying natural DNA or RNA polynucleotides in the presence of modified NTPs using an appropriate polymerase.

The nucleotides in the polynucleotide may be modified. The nucleotides may be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified with a label or a tag.

A single-stranded polynucleotide may contain regions with strong secondary structures, such as hairpins, quadruplexes, or triplex DNA. Structures of these types can be used to control the movement of the polynucleotide with respect to the nanopore. For example, secondary structures can be used to pause the movement of the polynucleotide through a nanopore, as described in more detail herein. Each successive secondary structure along the strand pauses the movement of the strand with respect to the nanopore as it is unwound and translocated. The polynucleotide may reform secondary structures after it has translocated through the nanopore. Such secondary structures can be used to prevent the polynucleotide from moving back through the nanopore under low or no applied negative voltages (applied to the trans side of the nanopore) and therefore assist in controlling the movement of the polynucleotide so it only occurs in a controlled manner in the relevant steps of the methods provided herein.

As used herein, a double stranded polynucleotide may comprise single stranded regions and regions with other structures, such as hairpin loops, triplexes and/or quadruplexes. Such secondary structures can be useful as described above in the context of single-stranded polynucleotides.

The two strands of the double-stranded molecule may be covalently linked, for example at the ends of the molecules by joining the 5′ end of one strand to the 3′ end of the other with a hairpin structure.

The target polynucleotide can be any length. For example, the target polynucleotide can be at least 10, at least 50, at least 100, at least 150, at least 200, at least 250, at least 300, at least 400 or at least 500 nucleotides or nucleotide pairs in length. The target polynucleotide can be 1000 or more nucleotides or nucleotide pairs, 5000 or more nucleotides or nucleotide pairs in length or 100000 or more nucleotides or nucleotide pairs in length or 500,000 or more nucleotides or nucleotide pairs in length, or 1,000,000 or more nucleotides or nucleotide pairs in length, 10, 000,000 or more nucleotides or nucleotide pairs in length, or 100,000,000 or more nucleotides or nucleotide pairs in length, or 200,000,000 or more nucleotides or nucleotide pairs in length, or the entire length of a chromosome.

The target polynucleotide may be an oligonucleotide. Oligonucleotides are short nucleotide polymers which typically have 50 or fewer nucleotides, such 40 or fewer, 30 or fewer, 20 or fewer, 10 or fewer or 5 or fewer nucleotides. The target oligonucleotide is preferably from about 15 to about 30 nucleotides in length, such as from about 20 to about 25 nucleotides in length. For example, the oligonucleotide can be about 15, about 16, about 17, about 18, about 19, about 20, about 21, about 22, about 23, about 24, about 25, about 26, about 27, about 28, about 29 or about 30 nucleotides in length.

The target polynucleotide may be a fragment of a longer polynucleotide. In this embodiment, the longer polynucleotide is typically fragmented into multiple, such as two or more, shorter polynucleotides.

The target polynucleotide may comprise the products of a PCR reaction, genomic DNA, the products of an endonuclease digestion and/or a DNA library.

The target polynucleotide may be naturally occurring. The target polynucleotide may be secreted from cells. Alternatively, the target analyte can be an analyte that is present inside cells such that the analyte must be extracted from the cells before the method can be carried out.

The target polynucleotide may be sourced from common organisms such as viruses, bacteria, archaea, plants or animals. Such organisms may be selected or altered to adjust the sequence of the target polynucleotide, for example by adjusting the base composition, removing unwanted sequence elements, and the like. The selection and alteration of organisms in order to arrive at desired polynucleotide characteristics is routine for one of ordinary skill in the art.

The source organism for the target polynucleotide may be chosen based on desired characteristics of the sequence. Desired characteristics include the ratio of single-stranded vs double-stranded polynucleotides produced by the organism; the complexity of the sequences of polynucleotides produced by the organism, the composition of the polynucleotides produced by the organism (such as the GC composition), or the length of contiguous polynucleotide strands produced by the organism. For example, when a contiguous polynucleotide strand of around 50 kb is required, lambda phage DNA can be used. If longer contiguous strands are required, other organisms can be used to produce the polynucleotide; for example E. coli produces around 4.5 Mb of contiguous dsDNA.

The target polynucleotide is often obtained from a human or animal, e.g. from urine, lymph, saliva, mucus, seminal fluid or amniotic fluid, or from whole blood, plasma or serum. The target polynucleotide may be obtained from a plant e.g. a cereal, legume, fruit or vegetable. The target polynucleotide may comprise genomic DNA. The genomic DNA may be fragmented. The DNA may be fragmented by any suitable method. For example, methods of fragmenting DNA are known in the art, Such methods may use a transposase, such as a MuA transposase. Often the genomic DNA is not fragmented.

In some embodiments the polynucleotide is synthetic or semi-synthetic. For example, DNA or RNA may be purely synthetic, synthesised by conventional DNA synthesis methods such as phosphoramidite based chemistries. Synthetic polynucleotides subunits may be joined together by known means, such as ligation or chemical linkage, to produce longer strands. In some embodiments internal self-forming structures (e.g. hairpins, quadruplexes) can be designed into the substrate e.g. by ligating appropriate sequences. Synthetic polynucleotides can be copied and scaled up for production by means known in the art, including PCR, incorporation into bacterial factories, and the like.

In some embodiments, the polynucleotide may have a simplified nucleotide composition. In some embodiments the polynucleotide has a repeating pattern of the same subunit. For example, a repeating unit may be (AmGn)q, wherein m, n and q are positive integers. For example, m is often from 1 to 20, such as from 1 to 10 e.g. from 1 to 5, e.g. 1, 2, 3, 4 or 5. n is often from 1 to 20, such as from 1 to 10 e.g. from 1 to 5, e.g. 1, 2, 3, 4 or 5. m and n may be the same or different. q is often from 1 to about 100,000. A typical repeating unit may be for example (AAAAAAGGGGGG)q. Repeating polynucleotides can be made by many means known in the art, for example by concatenating together synthetic subunits with sticky ends that enable ligation. In some embodiments the polynucleotide may therefore be a concatenated polynucleotide. Methods of concatenating polynucleotides are described in PCT/GB2017/051493.

In some embodiments, the polynucleotide can comprise bases which contain a reactive side-chain. Any suitable reactive functional groups can be incorporated on the side chain as required. Suitable examples of reactive functional groups include click chemistry reagents. Suitable examples of click chemistry include, but are not limited to, the following:
(a) copper-free variant of the 1,3 dipolar cycloaddition reaction, where an azide reacts with an alkyne under strain, for example in a cyclooctane ring;
(b) the reaction of an oxygen nucleophile on one linker with an epoxide or aziridine reactive moiety on the other; and
(c) the Staudinger ligation, where the alkyne moiety can be replaced by an aryl phosphine, resulting in a specific reaction with the azide to give an amide bond.

### Polynucleotide Adapter

In some embodiments, the motor protein and/or polynucleotide binding protein if present may be provided on a polynucleotide adapter. WO 2015/110813 describes the loading of motor proteins onto a target polynucleotide such as an adapter, and is hereby incorporated by reference in its entirety.

An adapter typically comprises a polynucleotide strand capable of being attached to the end of a target polynucleotide. The target polynucleotide is typically intended for characterisation in accordance with methods disclosed herein.

A polynucleotide adapter may be added to both ends of the target polynucleotide. Alternatively, different adapters may be added to the two ends of the target polynucleotide. An adapter may be added to just one end of the target polynucleotide. Methods of adding adapters to polynucleotides are known in the art. Adapters may be attached to polynucleotides, for example, by ligation, by click chemistry, by tagmentation, by topoisomerisation or by any other suitable method.

An adapter may be synthetic or artificial. Typically, an adapter comprises a polymer as described herein. In some embodiments, the adapter comprises a polynucleotide. In some embodiments an adapter may comprise a single-stranded polynucleotide strand. In some embodiments an adapter may comprise a double-stranded polynucleotide. A polynucleotide adapter may comprise DNA, RNA, modified DNA (such as a basic DNA), RNA, PNA, LNA, BNA and/or PEG. Usually, the adapter comprises single stranded and/or double stranded DNA or RNA.

An adapter may comprise a stalling moiety as described herein. The adapter may comprise a loading site for a motor protein or polynucleotide binding protein. The adapter may comprise a tag.

An adapter may be a Y adapter. A Y adapter is typically double stranded and comprises (a) at one end, a region where the two strands are hybridised together and (b), at the other end, a region where the two strands are not complementary. The non-complementary parts of the strands form overhangs. The hybridised stem of the adapter typically attaches to the 5′ end of a first strand of a double-stranded polynucleotide and the 3′ end of a second strand of a double-stranded polynucleotide; or to the 3′ end of a first strand of a double-stranded polynucleotide and the 5′ end of a second strand of a double-stranded polynucleotide. The presence of a non-complementary region in the Y adapter gives the adapter its Y shape since the two strands typically do not hybridise to each other unlike the double stranded portion. A motor protein or polynucleotide binding protein may bind to an overhang of an adapter such as a Y adapter. In another embodiment, a motor protein or polynucleotide binding protein may bind to the double stranded region. In other embodiments, a motor protein or polynucleotide binding protein may bind to a single-stranded and/or a double-stranded region of the adapter. In other embodiments, a first motor protein or polynucleotide binding protein may bind to the single-stranded region of such an adapter and a second motor protein or polynucleotide binding protein may bind to the double-stranded region of the adapter.

In one embodiment the adapter comprises a membrane anchor or a pore anchor. In some embodiments, the anchor may be attached to a polynucleotide that is complementary to and hence that is hybridised to the overhang to which a motor protein or polynucleotide binding protein is bound.

In some embodiments, one of the non-complementary strands of a polynucleotide adapter such as a Y adapter may comprise a leader sequence, which when contacted with a transmembrane pore is capable of threading into a nanopore.

The leader sequence typically comprises a polymer such as a polynucleotide, for instance DNA or RNA, a modified polynucleotide (such as abasic DNA), PNA, LNA, polyethylene glycol (PEG) or a polypeptide. In some embodiments, the leader sequence comprises a single strand of DNA, such as a poly dT section. The leader sequence can be any length, but is typically 10 to 150 nucleotides in length, such as from 20 to 120, 30 to 100, 40 to 80 or 50 to 70 nucleotides in length.

In one embodiment, a polynucleotide adapter is a hairpin loop adapter. A hairpin loop adapter is an adapter comprising a single polynucleotide strand, wherein the ends of the polynucleotide strand are capable of hybridising to each other, or are hybridized to each other, and wherein the middle section of the polynucleotide forms a loop. Suitable hairpin loop adapters can be designed using methods known in the art. Typically, the 3′ end of a hairpin loop adapter attaches to the 5′ end of a first strand of a double-stranded polynucleotide and the 5′ end of the hairpin loop adapter attaches to the 3′ end of a second strand of a double-stranded polynucleotide; or the 5′ end of a hairpin loop adapter attaches to the 3′ end of a first strand of a double-stranded polynucleotide and the 3′ end of the hairpin loop adapter attaches to the 5′ end of a second strand of a double-stranded polynucleotide. As explained in more detail below, a polynucleotide adapter can be attached to a target polynucleotide in order to characterise the target polynucleotide.

Those skilled in the art will also appreciate that when the adapter comprises a polynucleotide strand, the sequence of the adapter is typically not determinative and can be controlled or chosen according to the motor protein and other experimental conditions such as any polynucleotide to be characterised. Exemplary sequences are provided solely by way of illustration in the examples. For example, the adapter may comprise a sequence such as one or more of SEQ ID NOs: 21-26 or 28-33, or a polynucleotide sequence having at least 20%, such as at least 30%, e.g. at least 40% such as at least 50%, e.g. at least 60% such as at least 70%, e.g. at least 80%, for example at least 90% e.g. at least 95% sequence similarity or identity to one or more of SEQ ID NOs: 21-26 or 28-33. The sequence of the adapter can typically be altered without negatively affecting the efficacy of the methods provided herein.

In some embodiments a polynucleotide adapter may comprise a loading site for loading the motor protein and/or polynucleotide binding protein. The loading site may be for instance a single-stranded region which can targeted by the motor protein or polynucleotide binding protein. The loading site may be a region of the polynucleotide adapter to which a exogenous polynucleotide strand comprising the motor protein or polynucleotide binding protein can bind in order to transfer the motor protein or polynucleotide binding protein to the polynucleotide to be assessed in the methods provided herein.

The motor protein used in the methods provided herein may thus be stalled on the polynucleotide adapter. In other embodiments the motor protein is stalled on the target polynucleotide but is not stalled on the polynucleotide adapter.

### Blocking Moiety

In some embodiments a blocking moiety may be used to prevent the motor protein from disengaging from the target polynucleotide.

In some embodiments the blocking moiety is comprised in the target polynucleotide. In some embodiments the blocking moiety is comprised in a polynucleotide adapter attached to the target polynucleotide. In some embodiments a polynucleotide adapter, e.g. a polynucleotide adapter as described herein comprises a blocking moiety.

A blocking moiety may be used to prevent the motor protein from disengaging from the target polynucleotide. For example, if the motor protein is present at the 3′ end of a polynucleotide strand in the target polynucleotide or polynucleotide adapter, the blocking moiety is typically positioned between the motor protein and the 3′ terminus of the strand. If the motor protein is present at the 5′ end of a polynucleotide strand in the target polynucleotide or polynucleotide adapter, the blocking moiety is typically positioned between the motor protein and the 5′ terminus of the strand.

For example, in some embodiments a polynucleotide adapter may comprise a first end comprising an attachment point for attaching to a target polynucleotide analyte, and a second end; and a motor protein may be stalled on the polynucleotide adapter in an orientation for processing the adapter in the direction of the attachment point. In such embodiments, a blocking moiety may be positioned between the motor protein and the second end of the adapter in order to prevent the motor protein from disengaging from the second end of the polynucleotide adapter.

For example, in some embodiments a polynucleotide adapter may comprise a 3′ end comprising an attachment point for attaching to a 5′ end of a target polynucleotide analyte, and a 5′ end; and a motor protein may be stalled on the polynucleotide adapter in an orientation for processing the adapter in the direction of the 3′ end, i.e. in the 5′→3′direction. In such embodiments, a blocking moiety may be positioned between the motor protein and the 5′ end of the adapter in order to prevent the motor protein from disengaging from the 5′ end of the polynucleotide adapter. In other embodiments a polynucleotide adapter may comprise a 5′ end comprising an attachment point for attaching to a 3′ end of a target polynucleotide analyte, and a 3′ end; and a motor protein may be stalled on the polynucleotide adapter in an orientation for processing the adapter in the direction of the 5′ end, i.e. in the 3′→5′direction. In such embodiments, a blocking moiety may be positioned between the motor protein and the 3′ end of the adapter in order to prevent the motor protein from disengaging from the 3′ end of the polynucleotide adapter.

In some embodiments the target polynucleotide comprises a leader sequence at a first end of the target polynucleotide and the motor protein is stalled at a second end of the target polynucleotide or on an adapter attached to the second end of the target polynucleotide; and the blocking moiety is positioned between the motor protein and the second end of the polynucleotide (i.e. the terminus of the polynucleotide at the second end of the polynucleotide) thereby preventing the motor protein from disengaging from the target polynucleotide at the second end of the target polynucleotide.

For example, in some embodiments the target polynucleotide comprises a leader sequence at the 5′ end of a first strand and the motor protein is stalled at the 3′ end of the first strand on an adapter attached to the 3′ end of the first strand of the target polynucleotide; and the blocking moiety is positioned between the motor protein and the 3′ terminus of the first strand of the polynucleotide thereby preventing the motor protein from disengaging from the target polynucleotide at the 3′ end of the first strand of the target polynucleotide. In other embodiments the target polynucleotide comprises a leader sequence at the 3′ end of a first strand and the motor protein is stalled at the 5′ end of the first strand on an adapter attached to the 5′ end of the first strand of the target polynucleotide; and the blocking moiety is positioned between the motor protein and the 5′ terminus of the first strand of the polynucleotide thereby preventing the motor protein from disengaging from the target polynucleotide at the 5′ end of the first strand of the target polynucleotide. Of course the polynucleotide adapter may be attached to a double-stranded polynucleotide or a single stranded polynucleotide. When the target polynucleotide is a double-stranded polynucleotide the blocking moiety is typically positioned on the same strand as the motor protein. If a motor protein is present on each strand of the double-stranded polynucleotide (e.g. when the double-stranded polynucleotide is rotationally symmetrical) a blocking moiety is typically present on each strand of the polynucleotide.

Any suitable blocking moiety can be used in the provided methods. Suitable blocking moieties include many of the same groups that can be used as pausing moieties as described herein. For example, a blocking moiety may comprise one or more of:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups; and
a polynucleotide binding protein.
   These elements are described in more detail herein in the context of pausing moieties.

### Spacers

In some embodiments, a polynucleotide or polynucleotide adapter may comprise one or more spacers, e.g. from one to about 10 spacers, e.g. from 1 to about 5 spacers, e.g. 1, 2, 3, 4 or 5 spacers. The spacer may comprise any suitable number of spacer units. A spacer typically provides an energy barrier which impedes movement of a polynucleotide binding protein. For example, a spacer may impede movement of a motor protein or polynucleotide binding protein by reducing the traction of the protein, e.g. using an abasic spacer. A spacer may physically block movement of the protein, for instance by introducing a bulky chemical group to physically impede the movement of the polynucleotide binding protein.

In some embodiments, one or more spacers are included in the polynucleotide or in a polynucleotide adapter to provide a distinctive signal when they pass through or across a nanopore. One or more spacers may be used to define or separate one or more regions of a polynucleotide; e.g. to separate an adapter from the target polynucleotide.

In some embodiments, a spacer may comprise a linear molecule, such as a polymer, e.g. a polypeptide or a polyethylene glycol (PEG). Typically, such a spacer has a different structure from the target polynucleotide. For instance, if the target polynucleotide is DNA, the or each spacer typically does not comprise DNA. In particular, if the target polynucleotide is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA) or a synthetic polymer with nucleotide side chains. In some embodiments, a spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units.

In some embodiments, a spacer may comprise one or more chemical groups, e.g. one or more pendant chemical groups. The one or more chemical groups may be attached to one or more nucleobases in a polynucleotide adapter. The one or more chemical groups may be attached to the backbone of a polynucleotide adapter. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

In some embodiments, a spacer may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

Suitable spacers can be designed or selected depending on the nature of the polynucleotide or polynucleotide adapter, the motor protein and the conditions under which the method is to be carried out.

### Tags

In some embodiments a polynucleotide or polynucleotide adapter may comprise a tag or tether. For example, a polynucleotide can bind to a tag on a nanopore, e.g., via its adaptor, and release at some point, e.g., during characterization of the polynucleotide by the nanopore. A strong non-covalent bond (e.g., biotin/avidin) is still reversible and can be useful in some embodiments of the methods described herein.

In some embodiments, the pair of pore tag and polynucleotide adaptor can be configured such that the binding strength or affinity of a binding site on the polynucleotide (e.g., a binding site provided by an anchor or a leader sequence of an adaptor or by a capture sequence within the duplex stem of an adaptor) to a tag on a nanopore is sufficient to maintain the coupling between the nanopore and polynucleotide until an applied force is placed on it to release the bound polynucleotide from the nanopore.

In some embodiments, the tags or tethers are uncharged. This can ensure that the tags or tethers are not drawn into the nanopore under the influence of a potential difference.

One or more molecules that attract or bind the polynucleotide or adaptor may be linked to the detector (e.g. the pore). Any molecule that hybridizes to the adaptor and/or target polynucleotide may be used. The molecule attached to the pore may be selected from a PNA tag, a PEG linker, a short oligonucleotide, a positively charged amino acid and an aptamer. Pores having such molecules linked to them are known in the art. For example, pores having short oligonucleotides attached thereto are disclosed in Howarka et al (2001) Nature Biotech. 19: 636-639 and WO 2010/086620, and pores comprising PEG attached within the lumen of the pore are disclosed in Howarka et al (2000) J. Am. Chem. Soc. 122(11): 2411-2416.

A short oligonucleotide attached to the detector (e.g. a transmembrane pore), which oligonucleotide comprises a sequence complementary to a sequence in the leader sequence or another single stranded sequence in the adaptor may be used to enhance capture of the target polynucleotide in the methods described herein.

In some embodiments, the tag or tether may comprise or be an oligonucleotide (e.g., DNA, RNA, LNA, BNA, PNA, or morpholino). The oligonucleotide (e.g., DNA, RNA, LNA, BNA, PNA, or morpholino) can have about 10-30 nucleotides in length or about 10-20 nucleotides in length. In some embodiments, the oligonucleotide (e.g., DNA, RNA, LNA, BNA, PNA, or morpholino) for use in the tag or tether can have at least one end (e.g., 3′- or 5′-end) modified for conjugation to other modifications or to a solid substrate surface including, e.g., a bead. The end modifiers may add a reactive functional group which can be used for conjugation. Examples of functional groups that can be added include, but are not limited to amino, carboxyl, thiol, maleimide, aminooxy, and any combinations thereof. The functional groups can be combined with different length of spacers (e.g., C3, C9, C12, Spacer 9 and 18) to add physical distance of the functional group from the end of the oligonucleotide sequence.

In some embodiments, the tag or tether may comprise or be a morpholino oligonucleotide. The morpholino oligonucleotide can have about 10-30 nucleotides in length or about 10-20 nucleotides in length. The morpholino oligonucleotides can be modified or unmodified. For example, in some embodiments, the morpholino oligonucleotide can be modified on the 3′ and/or 5′ ends of the oligonucleotides. Examples of modifications on the 3′ and/or 5′ end of the morpholino oligonucleotides include, but are not limited to 3′ affinity tag and functional groups for chemical linkage (including, e.g., 3′-biotin, 3′-primary amine, 3′-disulfide amide, 3′-pyridyl dithio, and any combinations thereof); 5′ end modifications (including, e.g., 5′-primary ammine, and/or 5′-dabcyl), modifications for click chemistry (including, e.g., 3′-azide, 3′-alkyne, 5′-azide, 5′-alkyne), and any combinations thereof.

In some embodiments, the tag or tether may further comprise a polymeric linker, e.g., to facilitate coupling to a detector e.g. a nanopore. An exemplary polymeric linker includes, but is not limited to polyethylene glycol (PEG). The polymeric linker may have a molecular weight of about 500 Da to about 10 kDa (inclusive), or about 1 kDa to about 5 kDa (inclusive). The polymeric linker (e.g., PEG) can be functionalized with different functional groups including, e.g., but not limited to maleimide, NHS ester, dibenzocyclooctyne (DBCO), azide, biotin, amine, alkyne, aldehyde, and any combinations thereof. In some embodiments, the tag or tether may further comprise a 1 kDa PEG with a 5′-maleimide group and a 3′-DBCO group. In some embodiments, the tag or tether may further comprise a 2 kDa PEG with a 5′-maleimide group and a 3′-DBCO group. In some embodiments, the tag or tether may further comprise a 3 kDa PEG with a 5′-maleimide group and a 3′-DBCO group. In some embodiments, the tag or tether may further comprise a 5 kDa PEG with a 5′-maleimide group and a 3′-DBCO group.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of a nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444.

In some embodiments where one or more tags or tethers are attached to a nanopore via cysteine linkage(s), the one or more cysteines can be introduced to one or more monomers that form the nanopore by substitution. In some embodiments, the nanopore may be chemically modified by attachment of (i) Maleimides including diabromomaleimides such as: 4-phenylazomaleinanil, 1.N-(2-Hydroxyethyl)maleimide, N-Cyclohexylmaleimide, 1.3-Maleimidopropionic Acid, 1.1-4-Aminophenyl-1H-pyrrole,2,5,dione, 1.1-4-Hydroxyphenyl-1H-pyrrole,2,5,dione, N-Ethylmaleimide, N-Methoxycarbonylmaleimide, N-tert-Butylmaleimide, N-(2-Aminoethyl)maleimide, 3-Maleimido-PROXYL, N-(4-Chlorophenyl)maleimide, 1-[4-(dimethylamino)-3,5-dinitrophenyl]-1H-pyrrole-2,5-dione, N-[4-(2-Benzimidazolyl)phenyl]maleimide, N-[4-(2-benzoxazolyl)phenyl]maleimide, N-(1-naphthyl)-maleimide, N-(2,4-xylyl)maleimide, N-(2,4-difluorophenyl)maleimide, N-(3-chloro-para-tolyl)-maleimide, 1-(2-amino-ethyl)-pyrrole-2,5-dione hydrochloride, 1-cyclopentyl-3-methyl-2,5-dihydro-1H-pyrrole-2,5-dione, 1-(3-aminopropyl)-2,5-dihydro-1H-pyrrole-2,5-dione hydrochloride, 3-methyl-1-[2-oxo-2-(piperazin-1-yl)ethyl]-2,5-dihydro-1H-pyrrole-2,5-dione hydrochloride, 1-benzyl-2,5-dihydro-1H-pyrrole-2,5-dione, 3-methyl-1-(3,3,3-trifluropropyl)-2,5-dihydro-1H-pyrrole-2,5-dione, 1-[4-(methylamino)cyclohexyl]-2,5-dihydro-1H-pyrrole-2,5-dione trifluroacetic acid, SMILES O═C1C═CC(═O)N1CC=2C═CN═CC2, SMILES O═C1C═CC(═O)N1CN2CCNCC2, 1-benzyl-3-methyl-2,5-dihydro-1H-pyrrole-2,5-dione, 1-(2-fluorophenyl)-3-methyl-2,5-dihydro 1H-pyrrole-2,5-dione, N-(4-phenoxyphenyl)maleimide, N-(4-nitrophenyl)maleimide (ii) lodocetamides such as: 3-(2-Iodoacetamido)-proxyl, N-(cyclopropylmethyl)-2-iodoacetamide, 2-iodo-N-(2-phenylethyl)acetamide, 2-iodo-N-(2,2,2-trifluoroethyl)acetamide, N-(4-acetylphenyl)-2-iodoacetamide, N-(4-(aminosulfonyl)phenyl)-2-iodoacetamide, N-(1,3-benzothiazol-2-yl)-2-iodoacetamide, N-(2,6-diethylphenyl)-2-iodoacetamide, N-(2-benzoyl-4-chlorophenyl)-2-iodoacetamide, (iii) Bromoacetamides: such as N-(4-(acetylamino)phenyl)-2-bromoacetamide, N-(2-acetylphenyl)-2-bromoacetamide, 2-bromo-n-(2-cyanophenyl)acetamide, 2-bromo-N-(3-(trifluoromethyl)phenyl)acetamide, N-(2-benzoylphenyl)-2-bromoacetamide, 2-bromo-N-(4-fluorophenyl)-3-methylbutanamide, N-Benzyl-2-bromo-N-phenylpropionamide, N-(2-bromo-butyryl)-4-chloro-benzenesulfonamide, 2-Bromo-N-methyl-N-phenylacetamide, 2-bromo-N-phenethyl-acetamide, 2-adamantan-1-yl-2-bromo-N-cyclohexyl-acetamide, 2-bromo-N-(2-methylphenyl)butanamide, Monobromoacetanilide, (iv) Disulphides such as: aldrithiol-2, aldrithiol-4, isopropyl disulfide, 1-(Isobutyldisulfanyl)-2-methylpropane, Dibenzyl disulfide, 4-aminophenyl disulfide, 3-(2-Pyridyldithio)propionic acid, 3-(2-Pyridyldithio)propionic acid hydrazide, 3-(2-Pyridyldithio)propionic acid N-succinimidyl ester, am6amPDP1-βCD and (v) Thiols such as: 4-Phenylthiazole-2-thiol, Purpald, 5,6,7,8-tetrahydro-quinazoline-2-thiol.

In some embodiments, the tag or tether may be attached directly to a nanopore or via one or more linkers. The tag or tether may be attached to the nanopore using the hybridization linkers described in WO 2010/086602. Alternatively, peptide linkers may be used. Peptide linkers are amino acid sequences. The length, flexibility and hydrophilicity of the peptide linker are typically designed such that it does not to disturb the functions of the monomer and pore. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

### Anchor

In one embodiment, a polynucleotide or polynucleotide adapter may comprise a membrane anchor or a transmembrane pore anchor. In one embodiment the anchor assists in the characterisation of a target polynucleotide in accordance with the methods disclosed herein. For example, a membrane anchor or transmembrane pore anchor may promote localisation of the selected polynucleotides around a nanopore.

The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol. The anchor may comprise thiol, biotin or a surfactant. In one aspect the anchor may be biotin (for binding to streptavidin), amylose (for binding to maltose binding protein or a fusion protein), Ni-NTA (for binding to poly-histidine or poly-histidine tagged proteins) or peptides (such as an antigen).

In one embodiment, the anchor comprises a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The adapter may hybridise to a complementary sequence on a circular polynucleotide linker. The one or more anchors or one or more linkers may comprise a component that can be cut or broken down, such as a restriction site or a photolabile group. The linker may be functionalised with maleimide groups to attach to cysteine residues in proteins. Suitable linkers are described in WO 2010/086602.

In one embodiment, the anchor is cholesterol or a fatty acyl chain. For example, any fatty acyl chain having a length of from 6 to 30 carbon atom, such as hexadecanoic acid, may be used. Examples of suitable anchors and methods of attaching anchors to adapters are disclosed in WO 2012/164270 and WO 2015/150786.

In another embodiment the anchor may consist or comprise a hydrophobic modification to the polynucleotide or polynucleotide adapter. The hydrophobic modification may comprise a modified phosphate group comprised within the polynucleotide or polynucleotide anchor. The hydrophobic modification may for example comprise a phosphorothioate such as a charge-neutralized alkyl-phosphorothioate (PPT) as described in Jones et al, J. Am. Chem. Soc. 2021, 143, 22, 8305, the entire contents of which are hereby incorporated by reference. Suitable alkyl groups include for example C₁-C₁₀ alkyl groups such as C₂—C₆ alkyl groups; e.g. methyl, ethyl, propyl, butyl, pentyl and hexyl groups. Incorporation of the charge-neutralized alkyl-phosphorothioate into a polynucleotide allows for the polynucleotide to anchor to a hydrophobic region such as a lipid bilayer.

### Detector

In the methods provided herein, the polynucleotide is moved with respect to a detector such as a nanopore. The detector may be selected from (i) a zero-mode waveguide, (ii) a field-effect transistor, optionally a nanowire field-effect transistor; (iii) an AFM tip; (iv) a nanotube, optionally a carbon nanotube; and (v) a nanopore. Preferably, the detector is a nanopore.

The polynucleotide may be characterised in the methods provided herein in any suitable manner. In one embodiment the polynucleotide is characterised by detecting an ionic current or optical signal as the polynucleotide moves with respect to a nanopore. This is described in more detail herein. The method is amenable to these and other methods of detecting polynucleotides.

In another non-limiting example, in one embodiment the polynucleotide is characterised by detecting the by-products of a polynucleotide-processing reaction, such as a sequencing by synthesis reaction. The method may thus involve detecting the product of the sequential addition of (poly)nucleotides by an enzyme such as a polymerase to the nucleic acid strand. The product may be a change in one or more properties of the enzyme such as in the conformation of the enzyme. Such methods may thus comprise subjecting an enzyme such as polymerase or a reverse transcriptase to a double-stranded polynucleotide under conditions such that the template-dependent incorporation of nucleotide bases into a growing oligonucleotide strand causes conformational changes in the enzyme in response to sequentially encountering template strand nucleic acid bases and/or incorporating template-specified natural or analog bases (i.e., an incorporation event), detecting the conformational changes in the enzyme in response to such incorporation events, and thereby detecting the sequence of the template strand. In such methods the polynucleotide strand may be moved in accordance with the methods provided herein. Such methods may involve detecting and/or measuring incorporation events using methods known to those skilled in the art, such as those described in US 2017/0044605.

In another embodiment, by-products may be labelled so that a phosphate labelled species is released upon the addition of a nucleotide to a synthesised nucleic acid strand that is complementary to the template strand, and the phosphate labelled species is detected e.g. using a detector as described herein. The polynucleotide being characterised in this way may be moved in accordance with the methods herein. Suitable labels may be optical labels that are detected using a nanopore, or a zero mode wave guide, or by Raman spectroscopy, or other detectors. Suitable labels may be non-optical labels that are detected using a nanopore, or other detectors.

In another approach, nucleoside phosphates (nucleotides) are not labelled and upon the addition of a nucleotide to a synthesised nucleic acid strand that is complementary to the template strand, a natural by-product species is detected. Suitable detectors may be ion-sensitive field-effect transistors, or other detectors.

These and other detection methods are suitable for use in the methods described herein. Any suitable measurements can be taken using a detector as the polynucleotide moves with respect to the detector.

### Nanopore

In embodiments of the disclosed methods wherein the detector is a nanopore, any suitable nanopore can be used. In one embodiment a nanopore is a transmembrane pore.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

In the methods provided herein, the nanopore typically has a first opening and a second opening. The first opening is typically the cis opening and the second opening is typically the trans opening. However in some embodiments the first opening is the trans opening and the second opening is the cis opening. The motor protein used in the methods provided herein is typically provided at the first opening of the nanopore and thus controls the movement of the target polynucleotide in the direction from the second opening of the nanopore towards the first opening of the nanopore.

Any transmembrane pore may be used in the methods provided herein. The pore may be biological or artificial. Suitable pores include, but are not limited to, protein pores, polynucleotide pores and solid state pores. The pore may be a DNA origami pore (Langecker et al., Science, 2012; 338: 932-936). Suitable DNA origami pores are disclosed in WO2013/083983.

In one embodiment, the nanopore is a transmembrane protein pore. A transmembrane protein pore is a polypeptide or a collection of polypeptides that permits hydrated ions, such as polynucleotide, to flow from one side of a membrane to the other side of the membrane. In the methods provided herein, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

In one embodiment, the nanopore is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

In one embodiment, the transmembrane protein pore comprises a barrel or channel through which the ions may flow. The subunits of the pore typically surround a central axis and contribute strands to a transmembrane (3-barrel or channel or a transmembrane α-helix bundle or channel.

Typically, the barrel or channel of the transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

In one embodiment, the nanopore is a transmembrane protein pore derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porn F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and a outer membrane proteins, such as WZA and ClyA toxin.

In one embodiment the nanopore is a transmembrane pore derived from or based on Msp, α-hemolysin (α-HL), lysenin, CsgG, ClyA, Sp1 or haemolytic protein fragaceatoxin C (FraC).

In one embodiment, the nanopore is a transmembrane protein pore derived from CsgG, e.g. from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591.

In one embodiment, the nanopore is a transmembrane pore derived from lysenin. Examples of suitable pores derived from lysenin are disclosed in WO 2013/153359.

In one embodiment, the nanopore is a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

In one embodiment, the nanopore is a transmembrane protein pore derived from Msp, e.g. from MspA. Examples of suitable pores derived from MspA are disclosed in WO 2012/107778.

In one embodiment, the nanopore is a transmembrane pore derived from or based on ClyA.

### Membrane

In the disclosed methods, the detector is typically a nanopore present in a membrane. Any suitable membrane may be used.

The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

In some embodiments, the membrane is one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In some embodiments, a lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The methods disclosed herein are typically carried out using (i) an artificial amphiphilic layer comprising a pore, (ii) an isolated, naturally-occurring lipid bilayer comprising a pore, or (iii) a cell having a pore inserted therein. The methods are typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### General Methods

As mentioned above, the methods provided herein may be operated using any suitable detector, and as such any suitable apparatus for detecting polynucleotides can be used.

The methods provided herein may in some embodiments be carried out using any apparatus that is suitable for transmembrane pore sensing. For example, the apparatus may comprise a chamber comprising an aqueous solution and a barrier that separates the chamber into two sections. The barrier may have an aperture in which a membrane containing a transmembrane pore is formed. Transmembrane pores are described herein.

The methods may be carried out using the apparatus described in WO 2008/102120, WO 2010/122293 or WO 00/28312. In brief. the binding of a molecule (e.g. a target polynucleotide) in the channel of a pore will have an effect on the open-channel ion flow through the pore, which is the essence of “molecular sensing” of pore channels. Variation in the open-channel ion flow can be measured using suitable measurement techniques by the change in electrical current. The degree of reduction in ion flow, as measured by the reduction in electrical current, is related to the size of the obstruction within, or in the vicinity of, the pore. Binding of a molecule of interest (e.g. the target polynucleotide) in or near the pore therefore provides a detectable and measurable event, thereby forming the basis of a “biological sensor”. Detecting the presence of biological molecules finds application in personalised drug development, medicine, diagnostics, life science research, environmental monitoring and in the security and/or the defence industry.

When used to characterize the polynucleotide, the presence, absence or one or more characteristics of the target polynucleotide are determined. The methods may be for determining the presence, absence or one or more characteristics of at least one target polynucleotide. The methods may concern determining the presence, absence or one or more characteristics of two or more target polynucleotide. The methods may comprise determining the presence, absence or one or more characteristics of any number of target polynucleotides, such as 2, 5, 10, 15, 20, 30, 40, 50, 100 or more target polynucleotides. Any number of characteristics of the one or more target polynucleotides may be determined, such as 1, 2, 3, 4, 5, 10 or more characteristics. Characteristics amenable to being detected in the methods provide herein include the identity or sequence of the polynucleotide, the length, of the polynucleotide, whether or not the polynucleotide is modified, etc. In some embodiments the methods provided herein are methods of sequencing a target polynucleotide. In some embodiments a polynucleotide sequence may be determined in real-time by aligning real-time signal or basecalling to known references. Exemplary methods of determining a polynucleotide sequence are described in WO 2016/059427, incorporated by reference herein.

When used to characterize the polynucleotide, the methods may involve measuring the ion current flow through the pore, typically by measurement of a current. Alternatively, the ion flow through the pore may be measured optically, such as disclosed by Heron et al: J. Am. Chem. Soc. 9 Vol. 131, No. 5, 2009. Therefore the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The methods may involve measuring an optical signal as described in Chen et al, Nature Communications (2018) 9:1733, the entire contents of which are hereby incorporated by reference. For example, a nanopore such as an optically engineered nanopore structure (e.g. a plasmonic nanoslit) may be used to locally enable single-molecule surface enhanced Raman spectroscopy (SERS) to allow the characterisation of the polynucleotide through direct Raman spectroscopic detection.

The methods may be carried out on a silicon-based array of wells where each array comprises 128, 256, 512, 1024, 2000, 3000, 4000, 6000, 10000, 12000, 15000 or more wells.

The methods may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

In some embodiments of the disclosed methods, in particular those methods which involve re-reading a target polynucleotide as described herein, the methods comprise providing a condition for promoting the unbinding of the target polynucleotide from the polynucleotide binding site of the motor protein and/or for retarding re-binding of the target polynucleotide to the polynucleotide binding site of the motor protein.

The methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt may be an alkaline earth metal salt such as calcium chloride (CaCl2). The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of binding/no binding to be identified against the background of normal current fluctuations.

In some embodiments providing said condition comprises providing a salt concentration so as to increase the rate of unbinding of the target polynucleotide from the polynucleotide binding site of the motor protein. In some embodiments providing said condition comprises providing a salt concentration so as to reduce the rate of re-binding of the target polynucleotide to the polynucleotide binding site of the motor protein. Determining a suitable salt concentration to promote unbinding of a target polynucleotide from the polynucleotide-binding site of a motor protein and/or for retarding re-binding is within the capacity of one skilled in the art in view of the disclosure herein.

In some embodiments providing said condition comprises providing an osmolarity so as to increase the rate of unbinding of the target polynucleotide from the polynucleotide binding site of the motor protein. In some embodiments providing said condition comprises providing an osmolarity so as to reduce the rate of re-binding of the target polynucleotide to the polynucleotide binding site of the motor protein. Determining a suitable osmolarity to promote unbinding of a target polynucleotide from the polynucleotide-binding site of a motor protein and/or for retarding re-binding is within the capacity of one skilled in the art in view of the disclosure herein.

The methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

In some embodiments providing said condition comprises increasing the temperature so as to increase the rate of unbinding of the target polynucleotide from the polynucleotide binding site of the motor protein. In some embodiments providing said condition comprises increasing the temperature so as to reduce the rate of re-binding of the target polynucleotide to the polynucleotide binding site of the motor protein. Without being bound in any way by theory, the inventors consider that increasing the temperature may promote re-reading by e.g. increasing the off-rate of the motor protein from the polynucleotide. Determining a suitable temperature to promote unbinding of a target polynucleotide from the polynucleotide-binding site of a motor protein and/or for retarding re-binding is within the capacity of one skilled in the art in view of the disclosure herein.

Examples of providing a condition to promote re-reading by providing a temperature for promoting re-reading are provided herein, e.g. see Example 11. In some embodiments, providing a condition for promoting the unbinding of the target polynucleotide from the polynucleotide binding site of the motor protein and/or for retarding re-binding of the target polynucleotide to the polynucleotide binding site of the motor protein may comprise providing a temperature of from about 20° C. to about 50° C. such as from about 30° C. to about 45° C. e.g. from about 34° C. to about 40° C., e.g. about 31, 32, 33, 34, 35, 36, 37, 38, or 39° C.

### Further Aspects of the Disclosed Methods

The following are further aspects of the disclosed methods:
1. A method of characterising a target polynucleotide, the method comprising:
(i) contacting a detector with the target polynucleotide having a motor protein bound thereto, wherein said target polynucleotide is bound to the motor protein at a polynucleotide binding site of the motor protein;
(ii) taking one or more measurements characteristic of the target polynucleotide as the motor protein controls the movement of the target polynucleotide in a first direction with respect to the detector;
(iii) unbinding the target polynucleotide from the polynucleotide binding site of the motor protein, such that the target polynucleotide moves in a second direction with respect to the detector;
(iv) re-binding the target polynucleotide to the polynucleotide binding site of the motor protein; and taking one or more measurements characteristic of the target polynucleotide as the motor protein controls the movement of the target polynucleotide in the first direction with respect to the detector;

thereby characterising the target polynucleotide.

2. A method according to aspect 1, comprising repeating steps (iii) and (iv) multiple times.
3. A method according to aspect 1 or 2, wherein in step (ii) the motor protein controls the movement of a first portion of the target polynucleotide in the first direction with respect to the detector; and in step (iv) the motor protein controls the movement of a second portion of the target polynucleotide in the first direction with respect to the detector; and wherein the first portion at least partially overlaps with the second portion.
4. A method according to any one of the preceding aspects, wherein the first portion is the same as the second portion.
5. A method according to any one of the preceding aspects, wherein in step (iii) the distance the target polynucleotide moves with respect to the detector is at least 100 nucleotides in length.
6. A method according to any one of the preceding aspects, wherein the detector is comprised in a structure having a first opening and a second opening, or comprises a transmembrane nanopore having a first opening and a second opening; and step (i) comprises contracting the first opening with the target polynucleotide.
7. A method according to aspect 6, wherein (i) the motor protein controls the movement of the target polynucleotide in the direction from the second opening to the first opening; and (ii) when the target polynucleotide is unbound from the polynucleotide binding site of the motor protein, the target polynucleotide moves in the direction from the first opening to the second opening.
8. A method according to any one of the preceding aspects, comprising applying a force across the detector, and wherein the motor protein controls the movement of the target polynucleotide with respect to the detector in the direction opposite to the applied force.
9. A method according to any one of the preceding aspects, wherein the detector comprises a transmembrane nanopore spanning a membrane having a cis side and a trans side, and:

(i) the first opening of the nanopore is at the cis side of the membrane and the second opening of the nanopore is at the trans side; the motor protein controls the movement of the target polynucleotide through the nanopore from the trans side to the cis side of the membrane; and when the target polynucleotide is unbound from the polynucleotide binding site of the motor protein, the target polynucleotide moves through the nanopore from the cis side to the trans side of the membrane; or

(ii) the first opening of the nanopore is at the trans side of the membrane and the second opening of the nanopore is at the cis side; the motor protein controls the movement of the target polynucleotide through the nanopore from the cis side to the trans side of the membrane; and when the target polynucleotide is unbound from the polynucleotide binding site of the motor protein, the target polynucleotide moves through the nanopore from the trans side to the cis side of the membrane

10. A method according to any one of the preceding aspects, wherein the target polynucleotide is attached to or comprises a leader configured to promote unbinding of the polynucleotide binding site of the motor protein from the target polynucleotide in the vicinity of the leader.
11. A method according to aspect 10, wherein the target polynucleotide unbinds from the polynucleotide binding site of the motor protein when the motor protein contacts the leader.
12. A method according to aspect 10 or aspect 11, wherein the motor protein has a lower affinity for the leader than for the nucleotides of the target polynucleotide.
13. A method according to any one of aspects 10 to 12, wherein the leader comprises a different type of nucleotide to the target polynucleotide.
14. A method according to any one of aspects 10 to 13, wherein (i) the target polynucleotide comprises deoxyribonucleotides (DNA) and the leader comprises one or more nucleotides lacking both nucleobase and sugar moieties (spacer moieties), ribonucleotides (RNA), peptide nucleotides (PNA), glycerol nucleotides (GNA), threose nucleotides (TNA), locked nucleotides (LNA), bridged nucleotides (BNA), abasic nucleotides or nucleotides having a modified phosphate linkage; or (ii) the target polynucleotide comprises ribonucleotides (RNA) and the leader comprises one or more nucleotides lacking both nucleobase and sugar moieties (spacer moities), deoxyribonucleotides (DNA), peptide nucleotides (PNA), glycerol nucleotides (GNA), threose nucleotides (TNA), locked nucleotides (LNA), bridged nucleotides (BNA), abasic nucleotides or nucleotides having a modified phosphate linkage.
15. A method according to any one of aspects 10 to 14, wherein the target polynucleotide comprises deoxyribonucleotides (DNA) and the leader comprises one or more spacer moieties and/or one or more ribonucleotides.
16. A method according to any one of the preceding aspects, wherein the target polynucleotide does not disengage from the motor protein.
17. A method according to any one of the preceding aspects, wherein the motor protein is modified to prevent the target polynucleotide disengaging from the target polynucleotide.
18. A method according to any one of the preceding aspects, wherein the motor protein is modified to promote unbinding of the target polynucleotide from the polynucleotide binding site of the motor protein and/or to retard re-binding of the target polynucleotide to the polynucleotide binding site of the motor protein.
19. A method according to aspect any one of the preceding aspects, wherein the motor protein is modified with a closing moiety for (i) topologically closing the polynucleotide binding site of the motor protein around the target polynucleotide and (ii) promoting unbinding of the target polynucleotide from the polynucleotide binding site of the motor protein and/or retarding re-binding of the target polynucleotide to the polynucleotide binding site of the motor protein
20. A method according to aspect 19, wherein the motor protein is modified to facilitate attachment of the closing moiety to the motor protein.
21. A method according to aspect 20, wherein the motor protein is modified by substituting at least one amino acid in the motor protein for cysteine or for a non-natural amino acid.
22. A method according to any one of aspects 19 to 21, wherein the closing moiety comprises a bifunctional crosslinker.
23. A method according to any one of aspects 19 to 22, wherein the closing moiety crosslinks two amino acid residues of the motor protein, wherein at least one amino acid crosslinked by the closing moiety is a cysteine or a non-natural amino acid.
24. A method according to any one of aspects 19 to 23, wherein the closing moiety has a length of from about 1 Å to about 100 Å.
25. A method according to any one of aspects 19 to 21, wherein the closing moiety comprises a bond, preferably a disulphide bond.
26. A method according to any one of aspects 19 to 24, wherein the closing moiety comprises a structure of formula [A-B-C], wherein A and C are each independently reactive functional groups for reacting with amino acid residues in the motor protein and B is a linking moiety.
27. A method according to aspect 26, wherein A and C are each independently a cysteine-reactive functional group.
28. A method according to aspect 26 or 27, wherein linking moiety B comprises a linear or branched, unsubstituted or substituted alkylene, alkenylene, alkynylene, arylene, heteroarylene, carbocyclylene or heterocyclylene moiety, which moiety is optionally interrupted by and/or terminated in one or more atoms or groups selected from O, N(R), S, C(O), C(O)NR, C(O)O, unsubstituted or substituted arylene, arylene-alkylene, heteroarylene, heteroarylene-alkylene, carbocyclylene, carbocyclylene-alkylene, heterocyclylene and heterocyclylene-alkylene; wherein R is selected from H, unsubstituted or substituted alkyl, and unsubstituted or substituted aryl.
29. A method according to any one of aspects 26 to 28, wherein linking moiety B comprises an alkylene, oxyalkylene or polyoxyalkylene group and/or wherein A and C are each maleimide groups.
30. A method according to any one of aspects 19 to 25 or 26 to 29, wherein the closing moiety has a length of from about 5 Å to about 50 Å.
31. A method according to any one of the preceding aspects, comprising providing a condition for promoting the unbinding of the target polynucleotide from the polynucleotide binding site of the motor protein and/or for retarding re-binding of the target polynucleotide to the polynucleotide binding site of the motor protein.
32. A method according to aspect 31, wherein providing said condition comprises increasing the temperature so as to increase the rate of unbinding of the target polynucleotide from the polynucleotide binding site of the motor protein.
33. A method according to aspect 31 or 32, wherein providing said condition comprises increasing the temperature so as to reduce the rate of re-binding of the target polynucleotide to the polynucleotide binding site of the motor protein.
34. A method according to any one of the preceding aspects, wherein the motor protein is a helicase.
These aspects relate to features described in more detail herein.

### Polynucleotide Adapters

Also provided are polynucleotide adapters comprising motor proteins. It will be understood that any of the polynucleotide adapters disclosed herein can be applied in the embodiments of the methods discussed herein and above.

In one embodiment, provided herein is a polynucleotide adapter having a first end comprising an attachment point for attaching to a double-stranded polynucleotide analyte, and a second end; wherein said polynucleotide adapter comprises (i) a motor protein stalled thereon in an orientation for processing the adapter in the direction of the attachment point, and (ii) a blocking moiety positioned between the motor protein and the second end of the adapter.

In one embodiment, the polynucleotide adapter is a polynucleotide adapter as described in more detailed herein. In one embodiment, the motor protein is a motor protein as described herein. In one embodiment the blocking moiety is a blocking moiety as described herein.

The motor protein is oriented to process the polynucleotide adapter in the direction towards an attachment point on the adapter for attaching to a double-stranded polynucleotide. The motor protein may be orientated on the polynucleotide adapter to control the movement of the target polynucleotide in the trans-to-cis direction.

The motor protein is oriented on the polynucleotide adapter to control the movement of the target polynucleotide with respect to a detector such as a nanopore in a direction towards the motor protein; i.e., out of the detector e.g. out of the nanopore as described in more detail herein.

In some embodiments the polynucleotide adapter comprises a stalling moiety as described herein. In some embodiments the polynucleotide adapter comprises a pausing moiety as described herein.

### Kit

Also provided are kits comprising polynucleotide adapters and motor proteins. It will be understood that any of the polynucleotide adapters disclosed herein can be applied in the embodiments of the kits discussed herein and above.

In one embodiment, provided is a kit for modifying a target polynucleotide, comprising a first polynucleotide adapter as provided herein; and a second adapter comprising a single-stranded leader sequence at a first end and an attachment point for attaching to a double-stranded polynucleotide analyte at a second end.

In some embodiments the second adapter is an adapter as described in more detail herein.

### System

Also provided are systems comprising polynucleotide adapters, motor proteins, and nanopores. It will be understood that any of the polynucleotide adapters disclosed herein can be applied in the embodiments of the systems discussed herein and above.

In one embodiment provided is a system for characterising a target double-stranded polynucleotide comprising:
a polynucleotide adapter comprising a stalling moiety and optionally a pausing moiety;
a nanopore for characterising the target polynucleotide as the target polynucleotide moves with respect to the nanopore; and
a motor protein for moving the double-stranded polynucleotide in a first direction relative to the nanopore

In one embodiment, the polynucleotide adapter is a polynucleotide adapter as described in more detailed herein. In one embodiment, the motor protein is a motor protein as described herein. In one embodiment the nanopore is a nanopore as described herein. The system may further comprise a membrane; control equipment; etc as defined herein.

It is to be understood that although particular embodiments, specific configurations as well as materials and/or molecules, have been discussed herein for methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

This example demonstrates the controlled translocation of a DNA polynucleotide strand through a nanopore using a DNA motor which unwinds dsDNA whilst it translocates 5′-3′ on ssDNA. The DNA motor was initially stalled on a Y-adapter ligated to the polynucleotide. The polynucleotide was translocated through the nanopore in distinct phases: (1) an enzyme-free phase, in which the 3′ end of the polynucleotide was captured by the nanopore, and the nanopore translocated and separated the duplex under positive applied potential until it reached the DNA motor stalled on the distal 5′ end; (2) a ‘de-stalling’ phase, in which the DNA motor initially could not move over the stall under positive bias but was activated (‘de-stalled’) by applying a reverse potential; (3) a DNA motor-controlled phase, in which the motor began to move the DNA 5′-3′ out of the nanopore against the applied potential; (4) upon reaching the end of the polynucleotide, a constant blockade level was seen that could be cleared by reversing the potential to eject the strand.

An asymmetric 3.6-kilobase double-stranded DNA analyte (a fragment of bacteriophage lambda DNA; SEQ ID NO: 20) was obtained by PCR and was end-repaired and dA-tailed by NEBNext end repair and NEBNext dA-tailing modules (New England Biolabs (NEB)) and USER digest, to generate a 3′ dA overhang at one end and leaving a 3′ AGGA overhang at the opposite end.

A Y-adapter was prepared by annealing DNA oligonucleotides (SEQ ID NO: 21, SEQ ID NO: 22). A DNA motor (Dda helicase) was loaded onto the adapter. Monomeric traptavidin was added to the adapter to bind to the 5′ biotin moiety as blocker, to (1) prevent diffusion of the DNA motor backwards off the 5′ end and (2) prevent unintentional capture of the 5′ end of the library by the nanopore.

The double-stranded DNA analyte was ligated to the dA-tailed end of the Y-adapter using LNB from Oxford Nanopore Technologies sequencing kit SKQ-LSK109 (also referred to herein as LSK-SQK109; see https://community.nanoporetech.com/protocols/gDNA-sqk-lsk109/v/gde_9063_v109_revt_14 aug2019 for details) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘DNA library’.

Electrical measurements were acquired on a FLO-MIN106 MinION flow cell and MinION Mk1b from Oxford Nanopore Technologies. To 1200 μL FB (from Oxford Nanopore Technologies sequencing kit (SQK-LSK109)), 50 nM of DNA tether was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library, 0.7 μL excess monomeric traptavidin (˜100 nM tetramer) and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

A custom sequencing script was prepared to control the applied potential as follows: 10 sec capture phase (+120 mV); 0.5 sec de-stalling phase (0 mV); 85.5 seconds sequencing (+120 mV); eject phase (varied between 0 mV and −120 mV, 1 sec; −120 mV, 3 sec). This sequence of applied potentials was repeated multiple times.

Raw data was collected in a bulk FASTS file using MinKNOW software (Oxford Nanopore Technologies).

FIG. 6 shows the adapter used in this Example. FIG. 7 shows the adapter ligated to a double-stranded polynucleotide analyte. FIG. 8 shows the experimental schematic in this Example showing the pattern of applied potentials required to capture, destall and characterise the polynucleotide analyte. FIG. 9 shows an example current vs. time trace for this Example. The data show the capture of the polynucleotide analyte by the nanopore, followed by the controlled, stepwise movement of DNA out of the nanopore after it is ‘de-stalled’ by reducing the applied potential to between 0 and −120 mV. Few enzyme-mediated events were recorded above a destall potential of −40 mV, suggesting that between 0 and −40 mV, the single-strand is retained in the nanopore during the destall phase.

### Example 2

This example demonstrates the controlled translocation of both strands of a DNA polynucleotide duplex through a nanopore using a DNA motor which unwinds dsDNA whilst it translocates 5′-3′ on ssDNA. The DNA motor was initially stalled on a Y-adapter ligated to the polynucleotide. The template and complement strands were linked together via a hairpin moiety. The polynucleotide was translocated through the nanopore in distinct phases: (1) an enzyme-free phase, in which the 3′ end of the polynucleotide was captured by the nanopore, and the nanopore translocated and separated the duplex, first passing the complement strand, then the template strand, under positive applied potential until it reached the DNA motor stalled on the distal 5′ end; (2) a ‘de-stalling’ phase, in which the DNA motor initially could not move over the stall under positive bias but was activated (‘de-stalled’) by applying a reverse potential; (3) a DNA motor-controlled phase, in which the motor began to move the DNA 5′-3′ out of the nanopore against the applied potential; the DNA motor initially moved over the template strand, passed over the hairpin, and moved over the complement strand; (4) upon reaching the end of the polynucleotide, a constant blockade level was seen that could be cleared by reversing the potential to eject the strand.

An asymmetric 3.6-kilobase double-stranded DNA analyte (a fragment of bacteriophage lambda DNA; SEQ ID NO: 20) was obtained by PCR and was end-repaired and dA-tailed by NEBNext end repair and NEBNext dA-tailing modules (New England Biolabs (NEB)) and USER digest, to generate a 3′ dA overhang at one end and leaving a 3′ AGGA overhang at the opposite end.

A Y-adapter was prepared by annealing DNA oligonucleotides (SEQ ID NO: 21; SEQ ID NO: 22). A DNA motor (Dda helicase) was loaded onto the adapter. Monomeric traptavidin was added to the adapter to bind to the 5′ biotin moiety as blocker, to (1) prevent diffusion of the DNA motor backwards off the 5′ end and (2) prevent unintentional capture of the 5′ end of the library by the nanopore.

A hairpin bearing 3′-TCCT overhang was prepared by heating a DNA oligonucleotide (SEQ ID NO: 23) at 1 μM to 95° C. for 2 min in duplex-annealing buffer (Integrated DNA Technologies, Inc.), followed by snap-cooling on wet ice.

The double-stranded DNA analyte and the hairpin were ligated to the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘DNA library’.

Electrical measurements were acquired on a FLO-MIN106 MinION flow cell and MinION Mk1b from Oxford Nanopore Technologies. To 1200 μL FB (from Oxford Nanopore Technologies sequencing kit (SQK-LSK109)), 50 nM of DNA tether was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library, 0.7 μL excess monomeric traptavidin (˜100 nM tetramer) and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

A custom sequencing script was prepared to control the applied potential as follows: 10 sec capture phase (+120 mV); 0.5 sec de-stalling phase (variable according to experiment, ranging from 0 mV to −120 mV); 85.5 seconds sequencing (+120 mV); eject phase (0 mV, 1 sec; −120 mV, 3 sec). This sequence of applied potentials was repeated multiple times. Raw data was collected in a bulk FASTS file using MinKNOW software (Oxford Nanopore Technologies).

FIG. 10 shows the components used in this Example: hairpin (A), adapter (B) and polynucleotide analyte (C); (D) shows all the components ligated together. FIG. 11 shows the experimental schematic in this Example showing the pattern of applied potentials required to capture, destall and characterise the hairpin-derivatised polynucleotide analyte. FIG. 12a shows several example current vs. time traces for this Example. The data show the capture of the polynucleotide analyte by the nanopore, followed by the controlled, stepwise movement of DNA out of the nanopore after it is ‘de-stalled’. The de-stall potential was varied between 0 mV and −120 mV; however, no enzyme-mediated events were seen beyond −60 mV, suggesting the hairpin folded in the trans compartment confers resistance to ejection during destalling up to a potential of −60 mV, and additional resistance compared to single-stranded DNA alone (per Example 1). FIG. 12b shows the assignment of states A through G to an example current trace from FIG. 11. When compared with Example 1, an additional state E is observed in FIG. 12b, which can be ascribed to enzyme-mediated movement of the complement portion of the polynucleotide out of the nanopore, immediately following template portion D.

### Example 3

This example demonstrates the controlled de-stalling of the DNA motor by an ‘active de-stalling’ process. One or both strands of a DNA polynucleotide duplex were passed through a nanopore using a DNA motor which unwinds dsDNA whilst it translocates 5′-3′ on ssDNA. The DNA motor was initially stalled on a Y-adapter ligated to the polynucleotide. Optionally, at the distal end of the polynucleotide, the template and complement strands were linked via a hairpin moiety; otherwise, template and complement strands were left unlinked by omitting the hairpin. The polynucleotide translocated through the nanopore in distinct phases: (1) an enzyme-free phase, in which the 3′ end of the polynucleotide was captured by the nanopore, and the nanopore translocated and separated the duplex until it reached the DNA motor stalled on the distal 5′ end; (2) an active ‘de-stalling’ phase, in which the DNA motor initially could not move over the stall under positive bias but was activated (‘de-stalled’) by repeatedly applying an eject potential, followed by a return to the sequencing potential; (3) a DNA motor-controlled phase, in which the motor began to move the DNA 5′-3′ out of the nanopore against the applied potential; and (4) upon reaching the end of the polynucleotide, a constant blockade level was seen that could be cleared by reversing the potential to eject the strand.

An asymmetric 3.6-kilobase double-stranded DNA analyte (a fragment of bacteriophage lambda DNA; SEQ ID NO: 20) was obtained by PCR and was end-repaired and dA-tailed by NEBNext end repair and NEBNext dA-tailing modules (New England Biolabs (NEB)) and USER digest, to generate a 3′ dA overhang at one end and leaving a 3′ AGGA overhang at the opposite end.

A symmetric 3.6-kilobase double-stranded DNA analyte (a fragment of bacteriophage lambda DNA; SEQ ID NO: 20) was obtained by PCR and end-repaired and dA-tailed by NEBNext end repair and NEBNext dA-tailing modules (New England Biolabs (NEB)) to generate 3′ dA overhangs at both ends.

A Y-adapter was prepared by annealing DNA oligonucleotides (SEQ ID NO: 21, SEQ ID NO: 22). A DNA motor (Dda helicase) was loaded onto the adapter. Monomeric traptavidin was added to the adapter to bind to the 5′ biotin moiety as blocker, to (1) prevent diffusion of the DNA motor backwards off the 5′ end and (2) prevent unintentional capture of the 5′ end of the library by the nanopore.

A hairpin bearing 3′-TCCT overhang was prepared by heating a DNA oligonucleotide (SEQ ID NO: 23) at 1 μM to 95° C. for 2 min in duplex-annealing buffer (Integrated DNA Technologies, Inc.), followed by snap-cooling on wet ice.

The symmetric double-stranded DNA analyte was ligated to the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘1D DNA library’.

The asymmetric double-stranded DNA analyte was ligated to the Y-adapter and the hairpin using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘2D DNA library’.

Electrical measurements were acquired on a FLO-MIN106 MinION flow cell and MinION Mk1b from Oxford Nanopore Technologies. To 1200 μL FB (from Oxford Nanopore Technologies sequencing kit (SQK-LSK109)), 50 nM of DNA tether was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the 1D library or 2D DNA library, 0.7 μL excess monomeric traptavidin (˜100 nM tetramer) and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

A custom sequencing script was prepared to control the applied potential using the active unblock circuit of the MinION. The sequencing voltage was set to 120 mV and the active unblock potential (the ‘active de-stalling’ phase; phase (2), as described above) was set to −12 mV for the 1D library and −48 mV for the 2D library. Classifications for the stall level and strand (sequencing) level were programmed into a configuration file in the MinKNOW instrument control software that enabled the detection of the stalled species and applied an unblock potential that would not cause full ejection of the strand, using knowledge of the static unblock potentials from Examples 1 and 2. The script functioned as follows: if MinKNOW detected that a strand was at the stall level, it would apply the unblock potential first for 5 seconds, then return to the sequencing potential of 120 mV to check five times for actively sequencing strands. If the stall level was still present, it would apply the unblock potential for a further 25 seconds, and repeat five times. A rest period of 3 seconds was incorporated between each unblock attempt. If upon returning the sequencing potential, MinKNOW detected an actively sequencing strand, it would stop attempting to unblock and apply only the sequencing potential. If this entire process did not yield an actively sequencing strand, MinKNOW would turn off the channel. Every 15 minutes, a “mux scan” was applied to reset the system, which globally unblocked all channels on the flow cell and checked for active nanopores at 120 mV.

Raw data was collected in a bulk FASTS file using MinKNOW software (Oxford Nanopore Technologies).

FIGS. 7 and 10, D show the polynucleotide analytes used in this Example. Preparation of these was described in Examples 1 and 2. FIG. 13 shows example current traces for the 1D DNA library (A) and 2D DNA library (B). The portions during which destall attempts were made are marked with asterisks. The data show that both the 1D and 2D libraries could be destalled using these methods, and that several attempts could be made iteratively to destall the enzyme and then check for enzyme-controlled movement of the polynucleotide out of the nanopore.

### Example 4

This example demonstrates how the duration of the signal from the initial, enzyme-free portion of DNA translocation (3′-5′) through a nanopore may be used to estimate the size of a double-stranded DNA molecule whose template and complement strands are joined by a hairpin moiety, before a 5′-3′ DNA motor on the distal end actively translocates the DNA strand out of the nanopore in the opposite direction. Additionally, this example shows how markers added to the hairpin may be used to demarcate the signal.

The DNA motor was initially stalled on a Y-adapter ligated to the polynucleotide. The template and complement strands were linked together via a hairpin moiety per Example 2. Optionally, the hairpin moiety contained a bulky fluorophore group or an abasic group, and/or an additional oligonucleotide was hybridised to the hairpin.

An asymmetric 3.6-kilobase double-stranded DNA analyte (a fragment of bacteriophage lambda DNA; SEQ ID NO: 20) was obtained by PCR using primers, one of which contained multiple dUTP bases, and was end-repaired and dA-tailed by NEBNext end repair and NEBNext dA-tailing modules (New England Biolabs (NEB)), followed by NEB USER digest, to generate a 3′ dA overhang at one end and leaving a 3′ AGGA overhang at the opposite end.

A random library of Escherichia coli double-stranded DNA was generated by ligating generic adapters to E. coli SCS110 DNA which had been sheared using a Covaris gTube to a shear size of ˜20 kb and amplifying by PCR. Fragments were end-repaired and dA-tailed by NEBNext end repair and NEBNext dA-tailing modules (New England Biolabs (NEB)) to generate 3′ dA overhangs at both ends.

A Y-adapter was prepared by annealing DNA oligonucleotides (SEQ ID NO: 21, SEQ ID NO: 22). A DNA motor (Dda helicase) was loaded onto the adapter. Monomeric traptavidin was added to the adapter to bind to the 5′ biotin moiety as blocker, to (1) prevent diffusion of the DNA motor backwards off the 5′ end and (2) prevent unintentional capture of the 5′ end of the library by the nanopore.

Hairpins bearing 3′-TCCT or 3′-T overhangs were prepared by heating DNAs SEQ ID NO: 24, SEQ ID NO: 25 or SEQ ID NO: 26 at 1 μM to 95° C. for 2 min in duplex-annealing buffer (Integrated DNA Technologies, Inc.), followed by snap-cooling on wet ice.

The asymmetric 3.6-kilobase double-stranded DNA analyte and the hairpin (SEQ ID NO: 24 or SEQ ID NO: 26) were ligated to the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘3.6 kb DNA library’.

The Escherichia coli double-stranded DNA and the hairpin (SEQ ID NO: 25) were ligated to the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘random E. coli test library’.

Electrical measurements were acquired on a FLO-MIN106 MinION flow cell and MinION Mk1b from Oxford Nanopore Technologies. To 1200 μL FB (from Oxford Nanopore Technologies sequencing kit (SQK-LSK109)), 50 nM of DNA tether was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of either the 3.6 kb library or the random E. coli test library, 0.7 μL excess monomeric traptavidin (˜100 nM tetramer) and 22.5 μL of LB from Oxford Nanopore Technologies sequencing kit (SQK-LSK109) were mixed, yielding “sequencing mix”. To a portion of the reactions, oligonucleotide SEQ ID NO: 27 was also added at 50 nM. 75 μL of the sequencing mix were added to a MinION flowcell via the SpotON flow cell port.

The two libraries were tested with different run scripts. The 3.6 kb library was run with a custom sequencing script to control the applied potential as follows: 10 sec capture phase (+120 mV); 0.5 sec de-stalling phase (−40 mV); 85.5 seconds sequencing (+120 mV); eject phase (0 mV, 1 sec; −120 mV, 3 sec). This sequence of applied potentials was repeated multiple times. The random E. coli test library was run with the custom active de-stalling script described in Example 3, with capture/sequencing voltage of 120 mV and eject voltage of −48 mV.

Raw data was collected in a bulk FASTS file using MinKNOW software (Oxford Nanopore Technologies).

FIG. 14 shows the hairpin and oligonucleotide combinations which were used in this Example. The 3.6 kb DNA library was used to first characterise the capture-phase signals. FIG. 15 shows a schematic of the intermediates that would be expected to be detected in electrical measurements of enzyme-free and enzyme-mediated translocation. By comparison with FIG. 11, two additional states A1 and A2, corresponding to a bulky group in the nanopore and the blocker oligonucleotide atop the nanopore respectively (shown in FIG. 15), would be expected during the initial enzyme-free capture. An additional state D1, corresponding to the enzyme translocating over a bulky group in the hairpin moiety, would be expected between the template (D) and complement (E) phases of enzyme-mediated translocation. FIGS. 16a through 16d show example traces for each hairpin-oligonucleotide combination. A hairpin-only moiety (FIG. 16a) exhibited a relatively flat, yet detectable capture phase (marked by an asterisk). Addition of an oligonucleotide hybridised to the hairpin moiety introduced an additional uptick intermediate (marked as A2 in FIG. 16b), and the three bulky fluorescein-dT bases introduced a downtick (marked as A1 in FIG. 16c). The combination of oligonucleotide hybridised to the hairpin and the fluorescein-dT bases introduced both types of signal (seen in FIG. 16d). The introduction of an additional signal enabled the duration of the enzyme-free capture/entry phase of the polynucleotide to be measured (denoted by an asterisk in FIGS. 16a-d).

Examples using the scheme shown in FIG. 16b (hairpin plus hybridised oligonucleotide) were used to measure the enzyme-free capture phases for a random E. coli test library (FIG. 16e). FIG. 16e, i shows simplified (event-fitted) raw data for four examples. A threshold of 60 pA was used to measure the enzyme-free capture duration between states A and A2, denoted by an asterisk. FIG. 16e, ii shows the duration of the enzyme-mediated translocation plotted against the capture duration for thirty molecules. Linear regression analysis shows that the enzyme-free capture duration is correlated with the enzyme-mediated strand duration, confirming that it is possible to estimate the size of a strand using this method before decoding its sequence.

### Example 5

This example demonstrates the controlled translocation of a DNA polynucleotide strand through a nanopore using a DNA motor which unwinds dsDNA whilst it translocates 5′-3′ on ssDNA. This example describes an alternative adapter configuration to those described in previous examples. The DNA motor was initially stalled on a Y-adapter ligated to the polynucleotide. The polynucleotide was translocated through the nanopore in distinct phases: (1) an enzyme-free phase, in which the 3′ end of the polynucleotide was captured by the nanopore, and the nanopore translocated and separated the duplex under positive applied potential until it reaches the DNA motor stalled on the distal 5′ end; (2) a ‘de-stalling’ phase, in which the DNA motor initially could not move over the stall under positive bias but was activated (‘de-stalled’) by applying a reverse potential; (3) a DNA motor-controlled phase, in which the motor began to move the DNA 5′-3′ out of the nanopore against the applied potential; (4) upon reaching the end of the polynucleotide, a constant blockade level was seen that could be cleared by reversing the potential to eject the strand.

A Y-adapter was prepared by annealing DNA oligonucleotides (SEQ ID NO: 28, SEQ ID NO: 29, SEQ ID NO: 30 and SEQ ID NO: 31). A DNA motor (Dda helicase) was loaded onto the adapter. Compared to the previous examples, the oligonucleotide SEQ ID NO: 31 replaced the function of the biotin-streptavidin complex: the oligonucleotide formed a duplex region behind the enzyme which both prevented the enzyme diffusing backwards off the 5′ end of the strand upon which it was loaded, and prevented the capture of the 5′ terminated strand by the nanopore. The oligonucleotide SEQ ID NO: 30 acted as a forwards blocker to stall the enzyme in solution. A schematic of this adapter is shown in FIG. 17a.

A symmetric 3.6-kilobase double-stranded DNA analyte (a fragment of bacteriophage lambda DNA; SEQ ID NO: 20) obtained by PCR and end-repaired and dA-tailed by NEBNext end repair and NEBNext dA-tailing modules (New England Biolabs (NEB)) to generate 3′ dA overhangs at both ends.

The double-stranded DNA analyte was ligated to the dA-tailed end of the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘DNA library’. A schematic of the library is shown in FIG. 17b.

Electrical measurements were acquired on a FLO-MIN106 MinION flow cell and MinION Mk1b from Oxford Nanopore Technologies. To 1170 μL FB, 30 μL of FLT (from Oxford Nanopore Technologies sequencing kit (SQK-LSK109) were added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

The custom sequencing script described in Example 3 was used to control the de-stalling of the enzyme, with a sequencing voltage of 120 mV and eject voltage of 12 mV. Raw data was collected in a bulk FASTS file using MinKNOW software (Oxford Nanopore Technologies).

FIG. 17c shows a schematic of the intermediate steps expected to be seen during the capture of the polynucleotide and destalling of the enzyme. Compared to Example 1, an additional intermediate (blocker strand atop the nanopore, followed by blocker removal by the nanopore; state B in FIG. 17c) would be expected. FIG. 17d shows a representative current-time trace (i). The boxed portion (ii) corresponds to the capture/entry phase. In the example shown, the enzyme was destalled at the second five-second destall attempt (D), and the enzyme controlled movement of the polynucleotide out of the nanopore during E (expanded in iii). The data show that it is possible (a) to replace the function of the biotin-traptavidin linkage described in Example 1 with an oligonucleotide ‘back-blocker’, and (b) for the enzyme blocker oligonucleotide to be present as a separate piece that is removed by the nanopore.

### Example 6

This example demonstrates the controlled translocation of a DNA polynucleotide strand through a nanopore using a DNA motor which unwinds dsDNA whilst it translocates 5′-3′ on ssDNA. The DNA motor was initially stalled on a Y-adapter ligated to the polynucleotide. Compared to previous examples, the Y-adapter contained an oligonucleotide bearing a leader with thirty 3′-terminal C3 spacer residues. The polynucleotide was translocated through the nanopore in distinct phases: (1) an enzyme-free phase, in which the 3′ end of the polynucleotide was captured by the nanopore, and the nanopore translocated and separated the duplex under positive applied potential until it reaches the DNA motor stalled on the distal 5′ end; (2) a ‘de-stalling’ phase, in which the DNA motor initially could not move over the stall under positive bias but was activated (‘de-stalled’) by applying a reverse potential; (3) a DNA motor-controlled phase, in which the motor began to move the DNA 5′-3′ out of the nanopore against the applied potential; (4) upon reaching the end of the polynucleotide, a constant blockade level, distinctly different from the poly(dT) level in previous examples was seen that could be cleared by reversing the potential to eject the strand; and occasionally (5) under the force due to the applied sequencing potential, the enzyme would spontaneously slip backwards, rebind to upstream DNA, and repeat from step (3).

A Y-adapter was prepared by annealing DNA oligonucleotides (SEQ ID NO: 28, SEQ ID NO: 33, SEQ ID NO: 30 and SEQ ID NO: 32). A DNA motor (Dda helicase) was loaded onto the adapter. The SEQ ID NO: 33 oligonucleotide contained the C3 spacer residues described above.

A seven-fragment DNA library was derived via digest of bacteriophage lambda DNA using SnaBI and BamHI restriction enzymes, and end-repaired and dA-tailed by NEBNext end repair and NEBNext dA-tailing modules (New England Biolabs (NEB)) to generate 3′ dA overhangs at both ends of each fragment.

The seven-fragment DNA library was ligated to the dA-tailed end of the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘DNA library’.

Electrical measurements were acquired on a FLO-MIN106 MinION flow cell and MinION Mk1b from Oxford Nanopore Technologies. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

The DNA library was run with a custom sequencing script to control the applied potential as follows: 55 sec capture phase (+120 mV); 5 sec de-stalling phase (−20 mV); 55 seconds sequencing (+120 mV); eject phase (0 mV, 1 sec; −120 mV, 3 sec). This sequence of applied potentials was repeated multiple times. Raw data was collected in a bulk FASTS file using MinKNOW software (Oxford Nanopore Technologies).

FIG. 18a shows a schematic of the experiment. Compared with FIG. 17c, this experiment introduces an additional ‘rereading’ step (RR), wherein the enzyme unbinds and slips back from the 3′ C3 (non-DNA) leader to an earlier position on the DNA strand (E) and translocates 5′ to 3′ once more, resulting in multiple reads of the same DNA strand. The open-pore level is not seen between the re-reads, meaning it is unlikely that the molecule was ejected from the nanopore. FIG. 18b shows an example current-time trace of a molecule that was read twice (i and ii). A Hidden Markov Model was trained to map the enzyme-controlled portions against a reference for each restriction fragment (FIG. 18c). The data show that the reads map to the same fragment in the reference, and examples were recorded that partly mapped two or three times, confirming that the strand was read multiple times.

### Example 7

This example demonstrates how applied voltage may be used to control the speed of translocation of a DNA polynucleotide strand through a nanopore using a DNA motor which unwinds dsDNA whilst it translocates 5′-3′ on ssDNA, counter to the force applied on DNA by the electric field.

A Y-adapter was prepared by annealing DNA oligonucleotides (SEQ ID NO: 28, SEQ ID NO: 33, SEQ ID NO: 30 and SEQ ID NO: 32). A DNA motor (Dda helicase) was loaded onto the adapter.

A seven-fragment library of bacteriophage lambda was prepared per Example 6. The library was ligated to the dA-tailed end of the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘DNA library’.

Electrical measurements were acquired on a FLO-MIN106 MinION flow cell and MinION Mk1b from Oxford Nanopore Technologies. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

The DNA library was run with a following custom sequencing script to control the applied potential as follows: 55 sec capture phase (+120 to +200 mV); 5 sec de-stalling phase (−20 mV); 55 seconds sequencing (+120 mV); eject phase (0 mV, 1 sec; −120 mV, 3 sec). This sequence of applied potentials was repeated multiple times. Raw data was collected in a bulk FASTS file using MinKNOW software (Oxford Nanopore Technologies).

The experimental scheme was described in FIG. 17c; in this example, the capture/sequencing voltage was varied between 120 mV and 200 mV. Data were mapped using the HMM model described in Example 6. FIGS. 19a-d show HMM mappings for the data for 16 example reads for the data collected at 120 mV, 140 mV and 160 mV. The mappings were used to estimate the speed of the enzyme during the enzyme-controlled translocation phase. At 120 mV, the median speed of the enzyme was 319 bp/s; at 140 mV it was 259 bp/s; and at 160 mV it was 196 bp/s. The data demonstrate that an increase in the applied potential may be used to reduce the speed of the enzyme, in theory to zero.

### Example 8

This example demonstrates how the duration of the signal from the initial, enzyme-free portion of DNA translocation (3′-5′) through a nanopore may be used to estimate the size of one strand of a double-stranded DNA molecule before it is fully characterised, based solely on the duration of the capture/entry phase.

A Y-adapter was prepared by annealing DNA oligonucleotides (SEQ ID NO: 28, SEQ ID NO: 33, SEQ ID NO: 30 and SEQ ID NO: 32). A DNA motor (Dda helicase) was loaded onto the adapter.

A 10 kb fragment was obtained from bacteriophage lambda by PCR. Bacteriophages lambda DNA (˜48 kb) and T4 DNA (˜169 kb) were obtained from commercial sources. These double-stranded analytes were end-repaired and dA-tailed by NEBNext end repair and NEBNext dA-tailing modules (New England Biolabs (NEB)) to generate 3′ dA overhangs at both ends of each fragment. Each sample was ligated (separately) to the dA-tailed end of the Y-adapter using LNB from Oxford Nanopore Technologies sequencing kit (LSK-SQK109) and T4 DNA Ligase (NEB). The samples were purified using Agencourt AMPure XP (Beckman Coulter) beads, with two washes with LFB from Oxford Nanopore Technologies sequencing kit (LSK-SQK109). The ligated substrates were eluted into 10 mM Tris-Cl, 50 mM NaCl (pH 8.0), yielding a ‘10 kb library’, a ‘lambda library’ and a ‘T4 library’.

Electrical measurements were acquired on a FLO-MIN106 MinION flow cell and MinION Mk1b from Oxford Nanopore Technologies. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

Data were collected using a custom script similar to that described in Example 3, with a capture/sequencing voltage of 120 mV.

FIG. 20a shows the experimental schematic, similar to that of Example 5 above (FIG. 17c). The enzyme-free capture phase was measured by hand as the asterisked period between the open-pore level (A) and stall level (C), shown in more detail in FIG. 20b, bottom panel. The capture phase is discernible via its distinct noise and median current level characteristics. The enzyme-mediated translocation time (E) was also measured. FIG. 20b shows representative current-time traces for each of the three libraries described above, acquired on separate flow cells. For example, the 10 kb library had an enzyme-free capture duration of 1.6 sec and enzyme-mediated translocation time of 35.3 sec. Though long captures were obtained for the T4 library, no full-length examples were recorded, possibly owing to the increased likelihood of encountering nicks in the strand. FIG. 20c shows a plot of the log of the capture duration (A to C) vs. the log of the enzyme-mediated translocation duration. From 31 examples, a linear correlation (R²=0.74) was obtained, confirming that it is possible to estimate the size of a strand using this method before decoding its sequence.

### Example 9

This example demonstrates how a native DNA analyte may be re-read multiple times using motor proteins having different linker lengths of the disulfide closure.

Y-adapters bearing a leader arm containing 30 C3 Spacer units were prepared by annealing four DNA oligonucleotides with sequences SEQ ID NO: 67, SEQ ID NO: 68, SEQ ID NO: 69, and SEQ ID NO: 70. A DNA motor (a Dda helicase) was loaded onto each adapter, and the disulfide closed via reaction with one of the following linkers: diamide (TMAD), BMOE (1,2-bismaleimidoethane), BMOP (1,3-bismaleimidopropane), BMB (1,4-bismaleimidobutane), BM(PEG)₂ (1,8-bismaleimido-diethyleneglycol) or BM(PEG)₃ (1,11-bismaleimido-triethyleneglycol).

E. coli K12 PCR DNA was obtained by extraction from E. coli cells using a Qiagen genomic tip kit, sheared to a ˜10 kb cutoff using a Covaris gTube, end-repaired and dA-tailed using an Ultra II end-repair and dA-tailing kit (New England Biolabs), ligated to PCR adapters (PCA; Oxford Nanopore Technologies), and PCR-amplified using LongAmp Taq. The resultant double-stranded analyte was end-repaired and dA-tailed by NEBNext end repair and NEBNext dA-tailing modules (New England Biolabs (NEB)) to generate 3′ dA overhangs at both ends of each fragment. The sample was ligated to the T overhang of the Y-adapter using LNB from Oxford Nanopore Technologies sequencing kit (LSK-SQK109) and T4 DNA Ligase. The samples were purified using Agencourt AMPure XP (Beckman Coulter) beads, with two washes with LFB from Oxford Nanopore Technologies sequencing kit (LSK-SQK109). The ligated substrates were eluted into elution buffer (EB) from the same kit, yielding a ‘DNA library’. DNA libraries were prepared separately using adapters carrying Dda helicases closed with the disulfide linkers as described above.

Electrical measurements were acquired on a custom MinION flow cell and MinION Mk1b from Oxford Nanopore Technologies into which CsgG nanopores were inserted. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

Data were collected using a custom script similar to that described in Example 3, with a capture/sequencing voltage of 180 mV, except that the motor protein was destalled by switching the voltage to zero by disconnecting the channel for 5 sec when the enzyme stall level was detected. The active unblock was set to trigger upon recognising block levels not related to the terminal C3 level, nor the strand, nor open pore, nor enzyme stall levels.

Strand-level events from single-channel data that occurred immediately after the C3 level (“C3”, as marked in FIG. 18b) were scored as potential re-reads (e.g., “ii”, as marked in FIG. 18b). These re-reads were confirmed by base calling and comparing the sequence of the re-read to the original read (e.g., “i”, as marked in FIG. 18b), which occurs after the open pore and destalling events, as described in Example 6. Events that were in the same read orientation and within the span of the original read were classed as re-reads. The re-reading efficiency was quantified in two ways: (i) the proportion of reads which dropback and re-read within 30 seconds of reaching the C3 leader, and (ii) the dropback distance, which is the length of the re-read, i.e. the distance the enzyme was pushed back from the C3 leader.

The table below shows the results from this experiment. The results demonstrate re-reading with all linkers tested, and show that an increase in the linker length resulted in an increase in the proportion of reads that have an accompanying re-read within 30 seconds of reaching the C3 leader.

| Disulfide | Number of | Proportion of reads | Median |
|---|---|---|---|
| linker | re-read | which dropback | dropback |
| chemistry | events in run | within 30 seconds | distance (bases) |
| TMAD | 2987 | 0.024 | 747 |
| BMOE | 6334 | 0.049 | 498 |
| BMOP | 547 | 0.155 | 477 |
| BMB | 42 | 0.069 | 529 |
| BM(PEG)₂ | 596 | 0.165 | 427 |

### Example 10

This example demonstrates how a native DNA analyte may be re-read multiple using adapters with different sequences of the leader encountered by a Dda helicase at the 3′ terminus of the sequenced strand.

Y-adapters bearing a leader arm bearing RNA or C3 leader chemistry were prepared by annealing four DNA oligonucleotides with sequences SEQ ID NO: 67, SEQ ID NO: 68, and SEQ ID NO: 69, and a leader oligonucleotide selected from SEQ ID NO: 70, SEQ ID NO: 71 and SEQ ID NO: 72. A DNA motor (a Dda helicase) was loaded onto each adapter, and the disulfide closed via reaction with 1,2-bismaleimidoethane (BMOE).

DNA libraries were prepared by ligating the above Y-adapter to E. coli DNA prepared as described in Example 9.

Electrical measurements were acquired on a custom MinION flow cell and MinION Mk1b from Oxford Nanopore Technologies into which CsgG nanopores were inserted. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

Data were collected using a custom script similar to that described in Example 3, with a capture/sequencing voltage of 180 mV, except that the motor protein was destalled by switching the voltage to zero by disconnecting the channel for 5 sec when the enzyme stall level was detected. The active unblock was set to trigger upon recognising block levels not related to the terminal C3 level, nor the strand, open pore or enzyme stall levels.

Re-reading events were scored per Example 9, with some exceptions: in instances where the leader contained RNA, re-reads occurred from strand level.

The table below shows the results from this experiment. The results demonstrate re-reading with all leader oligonucleotides tested, and show that an optimum re-reading efficiency was obtained when leader oligonucleotide SEQ ID NO: 72 was used, as judged by a reduction in median time between re-reads.

| Leader | Number of | Median time | Median |
|---|---|---|---|
| oligonucleotide | re-read | between | dropback |
| in adapter | events in run | re-reads (s) | distance (bases) |
| SEQ ID NO: 70 | 209 | 104.0 | 817 |
| SEQ ID NO: 71 | 1719 | 122.6 | 812 |
| SEQ ID NO: 72 | 4016 | 79.4 | 724 |

### Example 11

This example demonstrates how a native DNA analyte may be re-read multiple times at a number of different sequencing run temperatures.

A Y-adapter bearing a leader arm bearing C3 leader chemistry was prepared by annealing four DNA oligonucleotides with sequences SEQ ID NO: 67, SEQ ID NO: 68, SEQ ID NO: 69 and SEQ ID NO: 70. A DNA motor (a Dda helicase) was loaded onto the adapter, and the disulfide closed via reaction with 1,2-bismaleimidoethane (BMOE).

DNA libraries were prepared by ligating the above Y-adapter to E. coli DNA prepared as described in Example 9.

Electrical measurements were acquired on a custom MinION flow cell and MinION Mk1b from Oxford Nanopore Technologies into which CsgG nanopores were inserted. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

Data were collected using a custom script similar to that described in Example 3, with a capture/sequencing voltage of 180 mV, except that the motor protein was destalled by switching the voltage to zero by disconnecting the channel for 5 sec when the enzyme stall level was detected. The active unblock was set to trigger upon recognising block levels not related to the terminal C3 level, nor the strand, open pore or enzyme stall levels. Re-reading events were scored per Example 9.

The table below shows the results from this experiment. The results demonstrate re-reading at all temperatures tested, and show that the re-reading efficiency increased as the temperature increased, as judged by increases in the proportion of reads that re-read within 30 seconds of reaching the C3 leader, and the median dropback distance.

| | % of strands that | |
|---|---|---|
| | re-read within 30 | |
| Run | sec of reaching | Median dropback |
| temperature | the C3 leader | distance (bases) |
| 34° C. | 5.3 | 467 |
| 37° C. | 9.1 | 584.5 |
| 40° C. | 11.6 | 787 |

### Description of the Sequence Listing

SEQ ID NO: 1 shows the amino acid sequence of (hexa-histidine tagged) exonuclease I (EcoExo I) from E. coli.

SEQ ID NO: 2 shows the amino acid sequence of the exonuclease III enzyme from E. coli.

SEQ ID NO: 3 shows the amino acid sequence of the RecJ enzyme from T. thermophilus (TthRecJ-cd).

SEQ ID NO: 4 shows the amino acid sequence of bacteriophage lambda exonuclease. The sequence is one of three identical subunits that assemble into a trimer. (neb.com/nebecomm/products/productM0262.asp).

SEQ ID NO: 5 shows the amino acid sequence of Phi29 DNA polymerase from Bacillus subtilis phage Phi29.

SEQ ID NO: 6 shows the amino acid sequence of Trwc Cba (Citromicrobium bathyomarinum) helicase.

SEQ ID NO: 7 shows the amino acid sequence of Hel308 Mbu (Methanococcoides burtonii) helicase.

SEQ ID NO: 8 shows the amino acid sequence of the Dda helicase 1993 from Enterobacteria phage T4.

SEQ ID NOs: 20-33 show the nucleotide sequences of DNA strands discussed in the examples.

SEQ ID NO: 40 shows the amino acid sequence of a preferred HhH domain.

SEQ ID NO: 41 shows the amino acid sequence of the ssb from the bacteriophage RB69, which is encoded by the gp32 gene.

SEQ ID NO: 42 shows the amino acid sequence of the ssb from the bacteriophage T7, which is encoded by the gp2.5 gene.

SEQ ID NO: 43 shows the amino acid sequence of the UL42 processivity factor from Herpes virus 1.

SEQ ID NO: 44 shows the amino acid sequence of subunit 1 of PCNA.

SEQ ID NO: 45 shows the amino acid sequence of subunit 2 of PCNA.

SEQ ID NO: 46 shows the amino acid sequence of subunit 3 of PCNA.

SEQ ID NO: 47 shows the amino acid sequence (from 1 to 319) of the UL42 processivity factor from the Herpes virus 1.

SEQ ID NO: 48 shows the amino acid sequence of the (HhH)2 domain.

SEQ ID NO: 49 shows the amino acid sequence of the (HhH)2-(HhH)2 domain.

SEQ ID NO: 50 shows the amino acid sequence of the human mitochondrial SSB (HsmtSSB).

SEQ ID NO: 51 shows the amino acid sequence of the p5 protein from Phi29 DNA polymerase.

SEQ ID NO: 52 shows the amino acid sequence of the wild-type SSB from E. coli. SEQ ID NO: 53 shows the amino acid sequence of the ssb from the bacteriophage T4, which is encoded by the gp32 gene.

SEQ ID NO: 54 shows the amino acid sequence of Topoisomerase V Mka (Methanopyrus kandleri).

SEQ ID NO: 55 shows the amino acid sequence of domains H-L of Topoisomerase V Mka (Methanopyrus kandleri).

SEQ ID NO: 56 shows the amino acid sequence of Mutant S (Escherichia coli).

SEQ ID NO: 57 shows the amino acid sequence of Sso7d (Sulfolobus solfataricus).

SEQ ID NO: 58 shows the amino acid sequence of Sso10b1 (Sulfolobus solfataricus P2).

SEQ ID NO: 59 shows the amino acid sequence of Sso10b2 (Sulfolobus solfataricus P2).

SEQ ID NO: 60 shows the amino acid sequence of Tryptophan repressor (Escherichia coli).

SEQ ID NO: 61 shows the amino acid sequence of Lambda repressor (Enterobacteria phage lambda).

SEQ ID NO: 62 shows the amino acid sequence of Cren7 (Histone crenarchaea Cren7 Sso).

SEQ ID NO: 63 shows the amino acid sequence of human histone (Homo sapiens).

SEQ ID NO: 64 shows the amino acid sequence of dsbA (Enterobacteria phage T4).

SEQ ID NO: 65 shows the amino acid sequence of Rad51 (Homo sapiens).

SEQ ID NO: 66 shows the amino acid sequence of PCNA sliding clamp (Citromicrobium bathyomarinum JL354).

SEQ ID NOs: 67 to 72 show the polynucleotide sequences of oligonucleotides described in Examples 9 to 11.

SEQUENCE LISTING

SEQ ID NO: 1-exonuclease 1 from E. coli
MMNDGKQQSTFLFHDYETFGTHPALDRPAQFAAIRTDSEFNVIGEPEVFYCKPADDYLPQ
PGAVLITGITPQEARAKGENEAAFAARIHSLFTVPKTCILGYNNVRFDDEVTRNIFYRNF
YDPYAWSWQHDNSRWDLLDVMRACYALRPEGINWPENDDGLPSFRLEHLTKANGIEHSNA
HDAMADVYATIAMAKLVKTRQPRLFDYLFTHRNKHKLMALIDVPQMKPLVHVSGMFGAWR
GNTSWVAPLAWHPENRNAVIMVDLAGDISPLLELDSDTLRERLYTAKTDLGDNAAVPVKL
VHINKCPVLAQANTLRPEDADRLGINRQHCLDNLKILRENPQVREKVVAIFAEAEPFTPS
DNVDAQLYNGFFSDADRAAMKIVLETEPRNLPALDITFVDKRIEKLLFNYRARNFPGTLD
YAEQQRWLEHRRQVFTPEFLQGYADELQMLVQQYADDKEKVALLKALWQYAEEIVSGSGH
HHHHH
SEQ ID NO: 2-exonuclease III enzyme from E. coli
MKFVSFNINGLRARPHQLEAIVEKHQPDVIGLQETKVHDDMFPLEEVAKLGYNVFYHGQK
GHYGVALLTKETPIAVRRGFPGDDEEAQRRIIMAEIPSLLGNVTVINGYFPQGESRDHPI
KFPAKAQFYQNLQNYLETELKRDNPVLIMGDMNISPTDLDIGIGEENRKRWLRTGKCSFL
PEEREWMDRLMSWGLVDTFRHANPQTADRFSWFDYRSKGFDDNRGLRIDLLLASQPLAEC
CVETGIDYEIRSMEKPSDHAPVWATFRR
SEQ ID NO: 3-RecJ enzyme from T. thermophilus
MFRRKEDLDPPLALLPLKGLREAAALLEEALRQGKRIRVHGDYDADGLTGTAILVRGLAA
LGADVHPFIPHRLEEGYGVLMERVPEHLEASDLFLTVDCGITNHAELRELLENGVEVIVT
DHHTPGKTPPPGLVVHPALTPDLKEKPTGAGVAFLLLWALHERLGLPPPLEYADLAAVGT
IADVAPLWGWNRALVKEGLARIPASSWVGLRLLAEAVGYTGKAVEVAFRIAPRINAASRL
GEAEKALRLLLTDDAAEAQALVGELHRLNARRQTLEEAMLRKLLPQADPEAKAIVLLDPE
GHPGVMGIVASRILEATLRPVFLVAQGKGTVRSLAPISAVEALRSAEDLLLRYGGHKEAA
GFAMDEALFPAFKARVEAYAARFPDPVREVALLDLLPEPGLLPQVFRELALLEPYGEGNP
EPLFL
SEQ ID NO: 4-bacteriophage lambda exonuclease
MTPDIILQRTGIDVRAVEQGDDAWHKLRLGVITASEVHNVIAKPRSGKKWPDMKMSYFHT
LLAEVCTGVAPEVNAKALAWGKQYENDARTLFEFTSGVNVTESPIIYRDESMRTACSPDG
LCSDGNGLELKCPFTSRDFMKFRLGGFEAIKSAYMAQVQYSMWVTRKNAWYFANYDPRMK
REGLHYVVIERDEKYMASFDEIVPEFIEKMDEALAEIGFVFGEQWR
SEQ ID NO: 5-Phi29 DNA polymerase
MKHMPRKMYSCAFETTTKVEDCRVWAYGYMNIEDHSEYKIGNSLDEFMAWVLKVQADLYF
HNLKFDGAFIINWLERNGFKWSADGLPNTYNTIISRMGQWYMIDICLGYKGKRKIHTVIY
DSLKKLPFPVKKIAKDFKLTVLKGDIDYHKERPVGYKITPEEYAYIKNDIQIIAEALLIQ
FKQGLDRMTAGSDSLKGFKDIITTKKFKKVFPTLSLGLDKEVRYAYRGGFTWLNDRFKEK
EIGEGMVFDVNSLYPAQMYSRLLPYGEPIVFEGKYVWDEDYPLHIQHIRCEFELKEGYIP
TIQIKRSRFYKGNEYLKSSGGEIADLWLSNVDLELMKEHYDLYNVEYISGLKFKATTGLF
KDFIDKWTYIKTTSEGAIKQLAKLMLNSLYGKFASNPDVTGKVPYLKENGALGFRLGEEE
TKDPVYTPMGVFITAWARYTTITAAQACYDRIIYCDTDSIHLTGTEIPDVIKDIVDPKKL
GYWAHESTFKRAKYLRQKTYIQDIYMKEVDGKLVEGSPDDYTDIKFSVKCAGMTDKIKKE
VTFENFKVGFSRKMKPKPVQVPGGVVLVDDTFTIKSGGSAWSHPQFEKGGGSGGGSGGSA
WSHPQFEK
SEQ ID NO: 6-Trwc Cba helicase
MLSVANVRSPSAAASYFASDNYYASADADRSGQWIGDGAKRLGLEGKVEARAFDALLRGE
LPDGSSVGNPGQAHRPGTDLTFSVPKSWSLLALVGKDERIIAAYREAVVEALHWAEKNAA
ETRVVEKGMVVTQATGNLAIGLFQHDTNRNQEPNLHFHAVIANVTQGKDGKWRTLKNDRL
WQLNTTLNSIAMARFRVAVEKLGYEPGPVLKHGNFEARGISREQVMAFSTRRKEVLEARR
GPGLDAGRIAALDTRASKEGIEDRATLSKQWSEAAQSIGLDLKPLVDRARTKALGQGMEA
TRIGSLVERGRAWLSRFAAHVRGDPADPLVPPSVLKQDRQTIAAAQAVASAVRHLSQREA
AFERTALYKAALDFGLPTTIADVEKRTRALVRSGDLIAGKGEHKGWLASRDAVVTEQRIL
SEVAAGKGDSSPAITPQKAAASVQAAALTGQGFRLNEGQLAAARLILISKDRTIAVQGIA
GAGKSSVLKPVAEVLRDEGHPVIGLAIQNTLVQMLERDTGIGSQTLARFLGGWNKLLDDP
GNVALRAEAQASLKDHVLVLDEASMVSNEDKEKLVRLANLAGVHRLVLIGDRKQLGAVDA
GKPFALLQRAGIARAEMATNLRARDPVVREAQAAAQAGDVRKALRHLKSHTVEARGDGAQ
VAAETWLALDKETRARTSIYASGRAIRSAVNAAVQQGLLASREIGPAKMKLEVLDRVNTT
REELRHLPAYRAGRVLEVSRKQQALGLFIGEYRVIGQDRKGKLVEVEDKRGKRFRFDPAR
IRAGKGDDNLTLLEPRKLEIHEGDRIRWTRNDHRRGLFNADQARVVEIANGKVTFETSKG
DLVELKKDDPMLKRIDLAYALNVHMAQGLTSDRGIAVMDSRERNLSNQKTFLVTVTRLRD
HLTLVVDSADKLGAAVARNKGEKASAIEVTGSVKPTATKGSGVDQPKSVEANKAEKELTR
SKSKTLDFGI
SEQ ID NO: 7-Hel308 Mbu helicase
MMIRELDIPRDIIGFYEDSGIKELYPPQAEAIEMGLLEKKNLLAAIPTASGKTLLAELAM
IKAIREGGKALYIVPLRALASEKFERFKELAPFGIKVGISTGDLDSRADWLGVNDIIVAT
SEKTDSLLRNGTSWMDEITTVVVDEIHLLDSKNRGPTLEVTITKLMRLNPDVQVVALSAT
VGNAREMADWLGAALVLSEWRPTDLHEGVLFGDAINFPGSQKKIDRLEKDDAVNLVLDTI
KAEGQCLVFESSRRNCAGFAKTASSKVAKILDNDIMIKLAGIAEEVESTGETDTAIVLAN
CIRKGVAFHHAGLNSNHRKLVENGFRQNLIKVISSTPTLAAGLNLPARRVIIRSYRRFDS
NFGMQPIPVLEYKQMAGRAGRPHLDPYGESVLLAKTYDEFAQLMENYVEADAEDIWSKLG
TENALRTHVLSTIVNGFASTRQELFDFFGATFFAYQQDKWMLEEVINDCLEFLIDKAMVS
ETEDIEDASKLFLRGTRLGSLVSMLYIDPLSGSKIVDGFKDIGKSTGGNMGSLEDDKGDD
ITVTDMTLLHLVCSTPDMRQLYLRNTDYTIVNEYIVAHSDEFHEIPDKLKETDYEWFMGE
VKTAMLLEEWVTEVSAEDITRHFNVGEGDIHALADTSEWLMHAAAKLAELLGVEYSSHAY
SLEKRIRYGSGLDLMELVGIRGVGRVRARKLYNAGFVSVAKLKGADISVLSKLVGPKVAY
NILSGIGVRVNDKHFNSAPISSNTLDTLLDKNQKTFNDFQ
SEQ ID NO: 8-Dda helicase
MTFDDLTEGQKNAFNIVMKAIKEKKHHVTINGPAGTGKTTLTKFIIEALISTGETGIILA
APTHAAKKILSKLSGKEASTIHSILKINPVTYEENVLFEQKEVPDLAKCRVLICDEVSMY
DRKLFKILLSTIPPWCTIIGIGDNKQIRPVDPGENTAYISPFFTHKDFYQCELTEVKRSN
APIIDVATDVRNGKWIYDKVVDGHGVRGFTGDTALRDFMVNYFSIVKSLDDLFENRVMAF
TNKSVDKLNSIIRKKIFETDKDFIVGEIIVMQEPLFKTYKIDGKPVSEIIFNNGQLVRII
EAEYTSTFVKARGVPGEYLIRHWDLTVETYGDDEYYREKIKIISSDEELYKFNLFLGKTA
ETYKNWNKGGKAPWSDFWDAKSQFSKVKALPASTFHKAQGMSVDRAFIYTPCIHYADVEL
AQQLLYVGVTRGRYDVFYV
SEQ ID NO: 20
GCCATCAGATTGTGTTTGTTAGTCGCTGCCATCAGATTGTGTTTGTTAGTCGCTTTTTTTTTTTGG
AATTTTTTTTTTGGAATTTTTTTTTTGCGCTAACAACCTCCTGCCGTTTTGCCCGTGCATATCGGT
CACGAACAAATCTGATTACTAAACACAGTAGCCTGGATTTGTTCTATCAGTAATCGACCTTATTCC
TAATTAAATAGAGCAAATCCCCTTATTGGGGGTAAGACATGAAGATGCCAGAAAAACATGACCTGT
TGGCCGCCATTCTCGCGGCAAAGGAACAAGGCATCGGGGCAATCCTTGCGTTTGCAATGGCGTACC
TTCGCGGCAGATATAATGGCGGTGCGTTTACAAAAACAGTAATCGACGCAACGATGTGCGCCATTA
TCGCCTAGTTCATTCGTGACCTTCTCGACTTCGCCGGACTAAGTAGCAATCTCGCTTATATAAGGA
GCGTGTTTATCGGCTACATCGGTACTGACTCGATTGGTTCGCTTATCAAACGCTTCGCTGGTAAAA
AAGCCGGAGTAGAAGATGGTAGAAATCAATAATCAACGTAAGGCGTTCCTCGATATGCTGGCGTGG
TCGGAGGGAACTGATAACGGACGTCAGAAAACCAGAAATCATGGTTATGACGTCATTGTAGGCGGA
GAGCTATTTACTGATTACTCCGATCACCCTCGCAAACTTGTCACGCTAAACCCAAAACTCAAATCA
ACAGGCGCCGGACGCTACCAGCTTCTTTCCCGTTGGTGGGATGCCTACCGCAAGCAGCTTGGCCTG
AAAGACTTCTCTCCGAAAAGTCAGGACGCTGTGGCATTGCAGCAGATTAAGGAGCGTGGCGCTTTA
CCTATGATTGATCGTGGTGATATCCGTCAGGCAATCGACCGTTGCAGCAATATCTGGGCTTCACTG
CCGGGCGCTGGTTATGGTCAGTTCGAGCATAAGGCTGACAGCCTGATTGCAAAATTCAAAGAAGGG
GGCGGAACGGTCAGAGAGATTGATGTATGAGCAGAGTCACCGGGATTATCTCCGCTCTGGTTATCT
GCATCATCGTCTGCCTGTCATGGGCTGTTAATCATTACCGTGATAACGCCATTACCTACAAAGCCC
AGCGCGACAAAAATGCCAGAGAACTGAAGCTGGCGAACGCGGCAATTACTGACATGCAGATGCGTC
AGCGTGATGTTGCTGCGCTCGATGCAAAATACACGAAGGAGTTAGCTGATGCTAAAGCTGAAAATG
ATGCTCTGCGTGATGATGTTGCCGCTGGTCGTCGTCGGTTGCACATCAAAGCAGTCTGTCAGTCAG
TGCGTGAAGCCACCACCGCCTCCGGCGTGGATAATGCAGCCTCCCCCCGACTGGCAGACACCGCTG
AACGGGATTATTTCACCCTCAGAGAGAGGCTGATCACTATGCAAAAACAACTGGAAGGAACCCAGA
AGTATATTAATGAGCAGTGCAGATAGAGTTGCCCATATCGATGGGCAACTCATGCAATTATTGTGA
GCAATACACACGCGCTTCCAGCGGAGTATAAATGCCTAAAGTAATAAAACCGAGCAATCCATTTAC
GAATGTTTGCTGGGTTTCTGTTTTAACAACATTTTCTGCGCCGCCACAAATTTTGGCTGCATCGAC
AGTTTTCTTCTGCCCAATTCCAGAAACGAAGAAATGATGGGTGATGGTTTCCTTTGGTGCTACTGC
TGCCGGTTTGTTTTGAACAGTAAACGTCTGTTGAGCACATCCTGTAATAAGCAGGGCCAGCGCAGT
AGCGAGTAGCATTTTTTTCATGGTGTTATTCCCGATGCTTTTTGAAGTTCGCAGAATCGTATGTGT
AGAAAATTAAACAAACCCTAAACAATGAGTTGAAATTTCATATTGTTAATATTTATTAATGTATGT
CAGGTGCGATGAATCGTCATTGTATTCCCGGATTAACTATGTCCACAGCCCTGACGGGGAACTTCT
CTGCGGGAGTGTCCGGGAATAATTAAAACGATGCACACAGGGTTTAGCGCGTACACGTATTGCATT
ATGCCAACGCCCCGGTGCTGACACGGAAGAAACCGGACGTTATGATTTAGCGTGGAAAGATTTGTG
TAGTGTTCTGAATGCTCTCAGTAAATAGTAATGAATTATCAAAGGTATAGTAATATCTTTTATGTT
CATGGATATTTGTAACCCATCGGAAAACTCCTGCTTTAGCAAGATTTTCCCTGTATTGCTGAAATG
TGATTTCTCTTGATTTCAACCTATCATAGGACGTTTCTATAAGATGCGTGTTTCTTGAGAATTTAA
CATTTACAACCTTTTTAAGTCCTTTTATTAACACGGTGTTATCGTTTTCTAACACGATGTGAATAT
TATCTGTGGCTAGATAGTAAATATAATGTGAGACGTTGTGACGTTTTAGTTCAGAATAAAACAATT
CACAGTCTAAATCTTTTCGCACTTGATCGAATATTTCTTTAAAAATGGCAACCTGAGCCATTGGTA
AAACCTTCCATGTGATACGAGGGCGCGTAGTTTGCATTATCGTTTTTATCGTTTCAATCTGGTCTG
ACCTCCTTGTGTTTTGTTGATGATTTATGTCAAATATTAGGAATGTTTTCACTTAATAGTATTGGT
TGCGTAACAAAGTGCGGTCCTGCTGGCATTCTGGAGGGAAATACAACCGACAGATGTATGTAAGGC
CAACGTGCTCAAATCTTCATACAGAAAGATTTGAAGTAATATTTTAACCGCTAGATGAAGAGCAAG
CGCATGGAGCGACAAAATGAATAAAGAACAATCTGCTGATGATCCCTCCGTGGATCTGATTCGTGT
AAAAAATATGCTTAATAGCACCATTTCTATGAGTTACCCTGATGTTGTAATTGCATGTATAGAACA
TAAGGTGTCTCTGGAAGCATTCAGAGCAATTGAGGCAGCGTTGGTGAAGCACGATAATAATATGAA
GGATTATTCCCTGGTGGTTGACTGATCACCATAACTGCTAATCATTCAAACTATTTAGTCTGTGAC
AGAGCCAACACGCAGTCTGTCACTGTCAGGAAAGTGGTAAAACTGCAACTCAATTACTGCAATGCC
CTCGTAATTAAGTGAATTTACAATATCGTCCTGTTCGGAGGGAAGAACGCGGGATGTTCATTCTTC
ATCACTTTTAATTGATGTATATGCTCTCTTTTCTGACGTTAGTCTCCGACGGCAGGCTTCAATGAC
CCAGGCTGAGAAATTCCCGGACCCTTTTTGCTCAAGAGCGATGTTAATTTGTTCAATCATTTGGTT
AGGAAAGCGGATGTTGCGGGTTGTTGTTCTGCGGGTTCTGTTCTTCGTTGACATGAGGTTGCCCCG
TATTCAGTGTCGCTGATTTGTATTGTCTGAAGTTGTTTTTACGTTAAGTTGATGCAGATCAATTAA
TACGATACCTGCGTCATAATTGATTATTTGACGTGGTTTGATGGCCTCCACGCACGTTGTGATATG
TAGATGATAATCATTATCACTTTACGGGTCCTTTCCGGTGAAAAAAAAGGTACCAAAAAAAACATC
GTCGTGAGTAGTGAACCGTAAGCATGTAGGA
SEQ ID NO: 21
/5BiotinTEG/TTTTTTTTTT/iSp18/AATGTACTTCGTTCAGTTACGTATTGCT
SEQ ID NO: 22
/5Phos/GCAATACGTAACTGAACGAAGT/iBNA-A//iBNA-MeC//iBNA-A//iBNA-T//
iBNA-T/TTTGAGGCGAGCGGTCAATTTTTTTTTTTTTTTTTTTT
SEQ ID NO: 23
/5Phos/TGCAATACGTAACTGAACGAAGTACATTAATGTACTTCGTTCAGTTACGTATTGCATCCT
SEQ ID NO: 24
/5Phos/TGCAATACGTAACTGAACGAAGTACATTTTTTTGAAGATAGAGCGATTTTTTTTTTTTT
TTTGTACTTCGTTCAGTTACGTATTGCATCCT
SEQ ID NO: 25
/5Phos/TGCAATACGTAACTGAACGAAGTACATTTTTTTGAAGATAGAGCGATTTTTTTTTTTTT
TTTGTACTTCGTTCAGTTACGTATTGCAT
SEQ ID NO: 26
/5Phos/TGCAATACGTAACTGAACGAAGTACATTTTTTTGAAGATAGAGCGATTTTT/iFluorT//
iFluorT//iFluorT/TTTTTTTTTGTACTTCGTTCAGTTACGTATTGCATCCT
SEQ ID NO: 27
/5BNA-T//iBNA-MeC//iBNA-G//iBNA-MeC//iBNA-T/CTATCTTC
SEQ ID NO: 28
GTTATTCAAGACTTCTTTAATACACTTTTTTTTTT/iSp18/AATGTACTTCGTTCAGTTACGTAT
TGCTTTGGCGTCTGCTTGGGTGTTTAACCT
SEQ ID NO: 29
/5Phos/GGTTAAACACCCAAGCAGACGCCTTTGAGGCGAGCGGTCAATTTTTTTTTTTTTTTTTTTT
SEQ ID NO: 30
GCAATACGTAACTGAACGAAGT/iBNA-A//iBNA-MeC//iBNA-A//iBNA-T//3BNA-T/
SEQ ID NO: 31
/5BNA-G//iBNA-T//iBNA-G//iBNA-T//iBNA-A/TTAAAGAAGTCTTGAAT/iBNA-A//
iBNA-A//3BNA-meC/
SEQ ID NO: 32
GTGTATTAAAGAAGTCTTGAATAAC
SEQ ID NO: 33
/5Phos/GGTTAAACACCCAAGCAGACGCCTTTGAGGCGAGCGGTCAA/iSpC3//iSpC3//
iSpC3//iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//iSpC3//iSpC3//3SpC3/
SEQ ID NO: 40
GTGSGAWKEWLERKVGEGRARRLIEYFGSAGEVGKLVENAEVSKLLEVPGIGDEAVARLV
PGGSS
SEQ ID NO: 41
MFKRKSTADLAAQMAKLNGNKGFSSEDKGEWKLKLDASGNGQAVIRFLPAKTDDALPFAI
LVNHGFKKNGKWYIETCSSTHGDYDSCPVCQYISKNDLYNTNKTEYSQLKRKTSYWANIL
VVKDPQAPDNEGKVFKYRFGKKIWDKINAMIAVDTEMGETPVDVTCPWEGANFVLKVKQV
SGFSNYDESKFLNQSAIPNIDDESFQKELFEQMVDLSEMTSKDKFKSFEELNTKFNQVLG
TAALGGAAAAAASVADKVASDLDDFDKDMEAFSSAKTEDDFMSSSSSDDGDLDDLLAGL
SEQ ID NO: 42
MAKKIFTSALGTAEPYAYIAKPDYGNEERGFGNPRGVYKVDLTIPNKDPRCQRMVDEIVK
CHEEAYAAAVEEYEANPPAVARGKKPLKPYEGDMPFFDNGDGTTTFKFKCYASFQDKKTK
ETKHINLVVVDSKGKKMEDVPIIGGGSKLKVKYSLVPYKWNTAVGASVKLQLESVMLVEL
ATFGGGEDDWADEVEENGYVASGSAKASKPRDEESWDEDDEESEEADEDGDF
SEQ ID NO: 43
MDSPGGVAPASPVEDASDASLGQPEEGAPCQVVLQGAELNGILQAFAPLRTSLLDSLLVM
GDRGILIHNTIFGEQVFLPLEHSQFSRYRWRGPTAAFLSLVDQKRSLLSVFRANQYPDLR
RVELAITGQAPFRTLVQRIWTTTSDGEAVELASETLMKRELTSFVVLVPQGTPDVQLRLT
RPQLTKVLNATGADSATPTTFELGVNGKFSVFTTSTCVTFAAREEGVSSSTSTQVQILSN
ALTKAGQAAANAKTVYGENTHRTFSVVVDDCSMRAVLRRLQVGGGTLKFFLTTPVPSLCV
TATGPNAVSAVFLLKPQKHHHHHH
SEQ ID NO: 44
MFKIVYPNAKDFFSFINSITNVTDSIILNFTEDGIFSRHLTEDKVLMAIMRIPKDVLSEY
SIDSPTSVKLDVSSVKKILSKASSKKATIELTETDSGLKIIIRDEKSGAKSTIYIKAEKG
QVEQLTEPKVNLAVNFTTDESVLNVIAADVTLVGEEMRISTEEDKIKIEAGEEGKRYVAF
LMKDKPLKELSIDTSASSSYSAEMFKDAVKGLRGFSAPTMVSFGENLPMKIDVEAVSGGH
MIFWIAPRLLE
SEQ ID NO: 45
MKAKVIDAVSFSYILRTVGDFLSEANFIVTKEGIRVSGIDPSRVVFLDIFLPSSYFEGFE
VSQEKEIIGFKLEDVNDILKRVLKDDTLILSSNESKLTLTFDGEFTRSFELPLIQVESTQ
PPSVNLEFPFKAQLLTITFADIIDELSDLGEVLNIHSKENKLYFEVIGDLSTAKVELSTD
NGTLLEASGADVSSSYGMEYVANTTKMRRASDSMELYFGSQIPLKLRFKLPQEGYGDFYI
APRAD
SEQ ID NO: 46
MKVVYDDVRVLKDIIQALARLVDEAVLKFKQDSVELVALDRAHISLISVNLPREMFKEYD
VNDEFKFGFNTQYLMKILKVAKRKEAIEIASESPDSVIINIIGSTNREFNVRNLEVSEQE
IPEINLQFDISATISSDGFKSAISEVSTVTDNVVVEGHEDRILIKAEGESEVEVEFSKDT
GGLQDLEFSKESKNSYSAEYLDDVLSLTKLSDYVKISFGNQKPLQLFFNMEGGGKVTYLL
APKVLE
SEQ ID NO: 47
TDSPGGVAPASPVEDASDASLGQPEEGAPCQVVLQGAELNGILQAFAPLRTSLLDSLLVM
GDRGILIHNTIFGEQVFLPLEHSQFSRYRWRGPTAAFLSLVDQKRSLLSVFRANQYPDLR
RVELAITGQAPFRTLVQRIWTTTSDGEAVELASETLMKRELTSFVVLVPQGTPDVQLRLT
RPQLTKVLNATGADSATPTTFELGVNGKFSVFTTSTCVTFAAREEGVSSSTSTQVQILSN
ALTKAGQAAANAKTVYGENTHRTFSVVVDDCSMRAVLRRLQVGGGTLKFFLTTPVPSLCV
TATGPNAVSAVFLLKPQK
SEQ ID NO: 48
WKEWLERKVGEGRARRLIEYFGSAGEVGKLVENAEVSKLLEVPGIGDEAVARLVP
SEQ ID NO: 49
WKEWLERKVGEGRARRLIEYFGSAGEVGKLVENAEVSKLLEVPGIGDEAVARLVPGYKTL
RDAGLTPAEAERVLKRYGSVSKVQEGATPDELRELGLGDAKIARILG
SEQ ID NO: 50
ESETTTSLVLERSLNRVHLLGRVGQDPVLRQVEGKNPVTIFSLATNEMWRSGDSEVYQLG
DVSQKTTWHRISVFRPGLRDVAYQYVKKGSRIYLEGKIDYGEYMDKNNVRRQATTIIADN
IIFLSDQTKEKE
SEQ ID NO: 51
ENTNIVKATFDTETLEGQIKIFNAQTGGGQSFKNLPDGTIIEANAIAQYKQVSDTYGDAK
EETVTTIFAADGSLYSAISKTVAEAASDLIDLVTRHKLETFKVKVVQGTSSKGNVFFSLQ
LSL
SEQ ID NO: 52
ASRGVNKVILVGNLGQDPEVRYMPNGGAVANITLATSESWRDKATGEMKEQTEWHRVVLF
GKLAEVASEYLRKGSQVYIEGQLRTRKWTDQSGQDRYTTEVVVNVGGTMQMLGGRQGGGA
PAGGNIGGGQPQGGWGQPQQPQGGNQFSGGAQSRPQQSAPAAPSNEPPMDFDDDIPF
SEQ ID NO: 53
MFKRKSTAELAAQMAKLNGNKGFSSEDKGEWKLKLDNAGNGQAVIRFLPSKNDEQAPFAI
LVNHGFKKNGKWYIETCSSTHGDYDSCPVCQYISKNDLYNTDNKEYSLVKRKTSYWANIL
VVKDPAAPENEGKVFKYRFGKKIWDKINAMIAVDVEMGETPVDVTCPWEGANFVLKVKQV
SGFSNYDESKFLNQSAIPNIDDESFQKELFEQMVDLSEMTSKDKFKSFEELNTKFGQVMG
TAVMGGAAATAAKKADKVADDLDAFNVDDFNTKTEDDFMSSSSGSSSSADDTDLDDLLND
L
SEQ ID NO: 54
MALVYDAEFVGSEREFEEERETFLKGVKAYDGVLATRYLMERSSSAKNDEELLELHQNFI
LLTGSYACSIDPTEDRYQNVIVRGVNFDERVQRLSTGGSPARYAIVYRRGWRAIAKALDI
DEEDVPAIEVRAVKRNPLQPALYRILVRYGRVDLMPVTVDEVPPEMAGEFERLIERYDVP
IDEKEERILEILRENPWTPHDEIARRLGLSVSEVEGEKDPESSGIYSLWSRVVVNIEYDE
RTAKRHVKRRDRLLEELYEHLEELSERYLRHPLTRRWIVEHKRDIMRRYLEQRIVECALK
LQDRYGIREDVALCLARAFDGSISMIATTPYRTLKDVCPDLTLEEAKSVNRTLATLIDEH
GLSPDAADELIEHFESIAGILATDLEEIERMYEEGRLSEEAYRAAVEIQLAELTKKEGVG
RKTAERLLRAFGNPERVKQLAREFEIEKLASVEGVGERVLRSLVPGYASLISIRGIDRER
AERLLKKYGGYSKVREAGVEELREDGLTDAQIRELKGLKTLESIVGDLEKADELKRKYGS
ASAVRRLPVEELRELGFSDDEIAEIKGIPKKLREAFDLETAAELYERYGSLKEIGRRLSY
DDLLELGATPKAAAEIKGPEFKFLLNIEGVGPKLAERILEAVDYDLERLASLNPEELAEK
VEGLGEELAERVVYAARERVESRRKSGRQERSEEEWKEWLERKVGEGRARRLIEYFGSAG
EVGKLVENAEVSKLLEVPGIGDEAVARLVPGYKTLRDAGLTPAEAERVLKRYGSVSKVQE
GATPDELRELGLGDAKIARILGLRSLVNKRLDVDTAYELKRRYGSVSAVRKAPVKELREL
GLSDRKIARIKGIPETMLQVRGMSVEKAERLLERFDTWTKVKEAPVSELVRVPGVGLSLV
KEIKAQVDPAWKALLDVKGVSPELADRLVEELGSPYRVLTAKKSDLMRVERVGPKLAERI
RAAGKRYVEERRSRRERIRRKLRG
SEQ ID NO: 55
SGRQERSEEEWKEWLERKVGEGRARRLIEYFGSAGEVGKLVENAEVSKLLEVPGIGDEAV
ARLVPGYKTLRDAGLTPAEAERVLKRYGSVSKVQEGATPDELRELGLGDAKIARILGLRS
LVNKRLDVDTAYELKRRYGSVSAVRKAPVKELRELGLSDRKIARIKGIPETMLQVRGMSV
EKAERLLERFDTWTKVKEAPVSELVRVPGVGLSLVKEIKAQVDPAWKALLDVKGVSPELA
DRLVEELGSPYRVLTAKKSDLMRVERVGPKLAERIRAAGKRYVEERRSRRERIRRKLRG
SEQ ID NO: 56
MSAIENFDAHTPMMQQYLRLKAQHPEILLFYRMGDFYELFYDDAKRASQLLDISLTKRGA
SAGEPIPMAGIPYHAVENYLAKLVNQGESVAICEQIGDPATSKGPVERKVVRIVTPGTIS
DEALLQERQDNLLAAIWQDSKGFGYATLDISSGRFRLSEPADRETMAAELQRTNPAELLY
AEDFAEMSLIEGRRGLRRRPLWEFEIDTARQQLNLQFGTRDLVGFGVENAPRGLCAAGCL
LQYAKDTQRTTLPHIRSITMEREQDSIIMDAATRRNLEITQNLAGGAENTLASVLDCTVT
PMGSRMLKRWLHMPVRDTRVLLERQQTIGALQDFTAGLQPVLRQVGDLERILARLALRTA
RPRDLARMRHAFQQLPELRAQLETVDSAPVQALREKMGEFAELRDLLERAIIDTPPVLVR
DGGVIASGYNEELDEWRALADGATDYLERLEVRERERTGLDTLKVGFNAVHGYYIQISRG
QSHLAPINYMRRQTLKNAERYIIPELKEYEDKVLTSKGKALALEKQLYEELFDLLLPHLE
ALQQSASALAELDVLVNLAERAYTLNYTCPTFIDKPGIRITEGRHPVVEQVLNEPFIANP
LNLSPQRRMLIITGPNMGGKSTYMRQTALIALMAYIGSYVPAQKVEIGPIDRIFTRVGAA
DDLASGRSTFMVEMTETANILHNATEYSLVLMDEIGRGTSTYDGLSLAWACAENLANKIK
ALTLFATHYFELTQLPEKMEGVANVHLDALEHGDTIAFMHSVQDGAASKSYGLAVAALAG
VPKEVIKRARQKLRELESISPNAAATQVDGTQMSLLSVPEETSPAVEALENLDPDSLTPR
QALEWIYRLKSLV
SEQ ID NO: 57
MATVKFKYKGEEKEVDISKIKKVWRVGKMISFTYDEGGGKTGRGAVSEKDAPKELLQMLE
KQKK
SEQ ID NO: 58
EKMSSGTPTPSNVVLIGKKPVMNYVLAALTLLNQGVSEIVIKARGRAISKAVDTVEIVRN
RFLPDKIEIKEIRVGSQVVTSQDGRQSRVSTIEIAIRKK
SEQ ID NO: 59
TEKLNEIVVRKTKNVEDHVLDVIVLFNQGIDEVILKGTGREISKAVDVYNSLKDRLGDGV
QLVNVQTGSEVRDRRRISYILLRLKRVY
SEQ ID NO: 60
AQQSPYSAAMAEQRHQEWLRFVDLLKNAYQNDLHLPLLNLMLTPDEREALGTRVRIVEEL
LRGEMSQRELKNELGAGIATITRGSNSLKAAPVELRQWLEEVLLKSD
SEQ ID NO: 61
MSTKKKPLTQEQLEDARRLKAIYEKKKNELGLSQESVADKMGMGQSGVGALFNGINALNA
YNAALLAKILKVSVEEFSPSIAREIYEMYEAVSMQPSLRSEYEYPVFSHVQAGMFSPELR
TFTKGDAERWVSTTKKASDSAFWLEVEGNSMTAPTGSKPSFPDGMLILVDPEQAVEPGDF
CIARLGGDEFTFKKLIRDSGQVFLQPLNPQYPMIPCNESCSVVGKVIASQWPEETFG
SEQ ID NO: 62
MSSGKKPVKVKTPAGKEAELVPEKVWALAPKGRKGVKIGLFKDPETGKYFRHKLPDDYPI
SEQ ID NO: 63
MARTKQTARKSTGGKAPRKQLATKAARKSAPATGGVKKPHRYRPGTVALREIRRYQKSTE
LLIRKLPFQRLVREIAQDFKTDLRFQSSAVMALQEASEAYLVGLFEDTNLCAIHAKRVTI
MPKDIQLARRIRGERA
SEQ ID NO: 64
MAKKEMVEFDEAIHGEDLAKFIKEASDHKLKISGYNELIKDIRIRAKDELGVDGKMFNRL
LALYHKDNRDVFEAETEEVVELYDTVFSK
SEQ ID NO: 65
MAMQMQLEANADTSVEEESFGPQPISRLEQCGINANDVKKLEEAGFHTVEAVAYAPKKEL
INIKGISEAKADKILAEAAKLVPMGFTTATEFHQRRSEIIQITTGSKELDKLLQGGIETG
SITEMFGEFRTGKTQICHTLAVTCQLPIDRGGGEGKAMYIDTEGTFRPERLLAVAERYGL
SGSDVLDNVAYARAFNTDHQTQLLYQASAMMVESRYALLIVDSATALYRTDYSGRGELSA
RQMHLARFLRMLLRLADEFGVAVVITNQVVAQVDGAAMFAADPKKPIGGNIIAHASTTRL
YLRKGRGETRICKIYDSPCLPEAEAMFAINADGVGDAKD
SEQ ID NO: 66
MKATIERATLLRCLSHVQSVVERRNTIPILSNVLIDADAGGGVKVMATDLDLQVVETMTA
ASVESAGAITVSAHLLFDIARKLPDGSQVSLETADNRMVVKAGRSRFQLPTLPRDDFPVI
VEGELPTSFELPARELAEMIDRTRFAISTEETRYYLNGIFLHVSDEARPVLKAAATDGHR
LARYTLDRPEGAEGMPDVIVPRKAVGELRKLLEEALDSNVQIDLSASKIRFALGGEGGVV
LTSKLIDGTFPDYSRVIPTGNDKLLRLDPKAFFQGVDRVATIATEKTRAVKMGLDEDKVT
LSVTSPDNGTAAEEIAAEYKAEGFEIGFNANYLKDILGQIDSDTVELHLADAGAPTLIRR
DENSPALYVLMPMRV
SEQ ID NO: 67
GTTATTCAAGACTTCTTTAATACACTTTTTTTTTT/iSp9/AATGTACTTCGTTCAGTTACGTATT
GCTTTGGCGTCTGCTTGGGTGTTTAACCT
SEQ ID NO: 68
GTGTATTAAAGAAGTCTTGAATAACTTTGAGGCGAGCGGTCAA
SEQ ID NO: 69
TTTGCAATACGTAACTGAACGAAGT/iBNA-A//iBNA-MeC//iBNA-A//iBNA-T//3BNA-T/
SEQ ID NO: 70
/5Phos/GGTTAAACACCCAAGCAGACGcCTTT/iSpC3//iSpC3//iSpC3//iSpC3//
iSpC3//iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//3SpC3/
SEQ ID NO: 71
/5Phos/GGTTAAACACCCAAGCAGACGCCTTTmUmUmUmUmUmU/iSpC3//iSpC3//iSpC3//
iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//iSpC3//iSpC3//3SpC3/
SEQ ID NO: 72
/5Phos/GGTTAAACACCCAAGCAGACGCCmUmU/iSpC3//iSpC3//iSpC3//iSpC3//
iSpC3//iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//iSpC3//iSpC3//iSpC3//
iSpC3//iSpC3//iSpC3//iSpC3//iSpC3//iSpC3//3SpC3/

1. A method of characterising a target polynucleotide, the method comprising:
(i) contacting the first opening of a transmembrane nanopore having a first opening and a second opening with the target polynucleotide; wherein the target polynucleotide has a motor protein stalled thereon; wherein the motor protein is stalled at a stalling moiety;
(ii) contacting the stalling moiety with the nanopore thereby destalling the motor protein; and
(iii) taking one or more measurements characteristic of the target polynucleotide as the motor protein controls the movement of the target polynucleotide through the nanopore in the direction from the second opening of the nanopore to the first opening of the nanopore;
thereby characterising the target polynucleotide.

2. A method according to claim 1, wherein the nanopore spans a membrane having a cis side and a trans side, and the first opening of the nanopore is at the cis side of the membrane and the second opening of the nanopore is at the trans side and the motor protein controls the movement of the target polynucleotide through the nanopore from the trans side to the cis side of the membrane.

3. A method according to claim 1, wherein the nanopore spans a membrane having a cis side and a trans side, and the first opening of the nanopore is at the trans side of the membrane and the second opening of the nanopore is at the cis side and the motor protein controls the movement of the target polynucleotide through the nanopore from the cis side to the trans side of the membrane.

4. A method according to any one of the preceding claims, comprising applying a force across the nanopore, and wherein the motor protein controls the movement of the target polynucleotide through the nanopore in the direction opposite to the applied force;
wherein said force preferably comprises a voltage potential applied across the nanopore.

5. A method according to any one of the preceding claims, wherein the motor protein is a helicase.

6. A method according to any one of the preceding claims, wherein the motor protein is a DNA-dependent ATPase (Dda) helicase.

7. A method according to any one of the preceding claims, wherein an adapter is attached to one or both ends of the target polynucleotide.

8. A method according to claim 7, wherein the motor protein is stalled on the adapter.

9. A method according to any one of the preceding claims, wherein the nanopore captures a leader sequence at a first end of the target polynucleotide and the motor protein is stalled at a second end of the target polynucleotide or on an adapter attached to the second end of the target polynucleotide.

10. A method according to any one of the preceding claims, wherein:
the target polynucleotide is single-stranded;
the target polynucleotide comprises a leader sequence, wherein the leader sequence is located at the first end of the target polynucleotide or is comprised in an adapter attached to the first end of the target polynucleotide; and
the motor protein is stalled at the second end of the target polynucleotide or is stalled on an adapter at the second end of the target polynucleotide.

11. A method according to any one of claims 1 to 9, wherein the target polynucleotide is double stranded.

12. A method according to claim 11 wherein:
the target polynucleotide is double-stranded and comprises a first strand and a second strand;
the target polynucleotide comprises a leader sequence, wherein the leader sequence is located at a first end of the polynucleotide and is comprised in the first strand or is comprised in an adapter attached to the first strand; and
the motor protein is stalled at a second end of the target polynucleotide.

13. A method according to claim 12, wherein the motor protein is stalled at the second end of the first strand of the target polynucleotide or is stalled on an adapter at the second end of the first strand of the target polynucleotide.

14. A method according to claim 12 or claim 13, wherein the first strand and the second strand are attached together by a hairpin adapter at the second end of the first strand; and the motor protein is stalled at the hairpin adapter.

15. A method according to claim 12, wherein the first strand and the second strand are attached together by a hairpin adapter attached to (i) the second end of the first strand and (ii) a first end of the second strand; and the motor protein is stalled at a second end of the second strand or is stalled on an adapter at the second end of the second strand of the double-stranded polynucleotide.

16. A method according to any one of the preceding claims, wherein the target polynucleotide comprises a portion which is complementary to a tag sequence.

17. A method according to any one of the preceding claims, wherein the target polynucleotide comprises a portion having an oligonucleotide hybridised thereto, and wherein the oligonucleotide comprises: (a) a hybridising portion for hybridising to the target polynucleotide and (b) (i) a portion complementary to a tag sequence or (ii) an affinity molecule capable of binding to a tag.

18. A method according to claim 16 or claim 17, wherein the target polynucleotide is double stranded and the portion which is complementary to a tag sequence is a portion of the first strand of the polynucleotide and/or the portion having an oligonucleotide hybridised thereto is a portion of the first strand of the polynucleotide.

19. A method according to any one of the preceding claims, wherein the motor protein is stalled at a stalling site comprising one or more stalling units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
spacer units selected from nitroindoles, inosines, acridines, 2-aminopurines, 2 diaminopurines, 5-bromo-deoxyuridines, inverted thymidines (inverted dTs), inverted dideoxy-thymidines (ddTs), dideoxy-cytidines (ddCs), 5-methylcytidines, 5-hydroxymethylcytidines, 2′-O-Methyl RNA bases, Iso-deoxycytidines (Iso-dCs), Iso-deoxyguanosines (Iso-dGs), C3 (OC₃H₆OPO₃) groups, photo-cleavable (PC) [OC₃H₆—C(O)NHCH₂—C₆H₃NO₂—CH(CH₃)OPO₃] groups, hexandiol groups, spacer 9 (iSp9) [(OCH₂CH₂)₃OPO₃] groups, spacer 18 (iSp18) [(OCH₂CH₂)₆OPO₃] groups; and thiol connections; and
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups.

20. A method according to any one of the preceding claims, wherein destalling the motor protein comprises applying a destalling force to the polynucleotide, wherein the destalling force is lower in magnitude and/or of opposite direction to a read force, wherein the read force is the force applied whilst the motor protein controls the movement of the target polynucleotide and the measurements to determine one or more characteristics of the polynucleotide are taken.

21. A method according to claim 20, wherein destalling the motor protein comprises stepping the applied force one or more times between the destalling force and the read force.

22. A method according to any one of the preceding claims, wherein the motor protein is stalled at a stalling site comprising one or more stalling units and one or more pausing moieties; and wherein contacting the one or more pausing moieties with the nanopore retards the movement of the polynucleotide through the nanopore thereby causing the motor protein to destall from the one or more stalling units.

23. A method according to claim 22, wherein the pausing moiety comprises one or more pausing units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups; and
a polynucleotide binding protein.

24. A method according to any one of the preceding claims, wherein the target polynucleotide comprises a blocking moiety to prevent the motor protein from disengaging from the polynucleotide.

25. A method according to claim 24, wherein the target polynucleotide comprises a leader sequence at a first end of the target polynucleotide and the motor protein is stalled at a second end of the target polynucleotide or on an adapter attached to the second end of the target polynucleotide; and the blocking moiety is positioned between the motor protein and the second end of the polynucleotide thereby preventing the motor protein from disengaging from the target polynucleotide at the second end of the target polynucleotide.

26. A polynucleotide adapter having a first end comprising an attachment point for attaching to a double-stranded polynucleotide analyte, and a second end;
wherein said polynucleotide adapter comprises (i) a motor protein stalled thereon in an orientation for processing the adapter in the direction of the attachment point, and (ii) a blocking moiety positioned between the motor protein and the second end of the adapter.

27. A kit, comprising a first adapter according to claim 26 and a second adapter comprising a single-stranded leader sequence at a first end and an attachment point for attaching to a double-stranded polynucleotide analyte at a second end.

28. A polynucleotide adapter or kit according to claim 26 or claim 27, wherein said polynucleotide adapter, said motor protein and/or said blocking moiety is as defined in any one of the preceding claims.

29. A method of characterising a target polynucleotide, the method comprising:
(i) contacting a detector with the target polynucleotide having a motor protein bound thereto, wherein said target polynucleotide is bound to the motor protein at a polynucleotide binding site of the motor protein;
(ii) taking one or more measurements characteristic of the target polynucleotide as the motor protein controls the movement of the target polynucleotide in a first direction with respect to the detector;
(iii) unbinding the target polynucleotide from the polynucleotide binding site of the motor protein, such that the target polynucleotide moves in a second direction with respect to the detector;
(iv) re-binding the target polynucleotide to the polynucleotide binding site of the motor protein; and taking one or more measurements characteristic of the target polynucleotide as the motor protein controls the movement of the target polynucleotide in the first direction with respect to the detector;
thereby characterising the target polynucleotide.

30. A method according to claim 29, comprising repeating steps (iii) and (iv) multiple times.

31. A method according to claim 29 or 30, wherein in step (ii) the motor protein controls the movement of a first portion of the target polynucleotide in the first direction with respect to the detector; and in step (iv) the motor protein controls the movement of a second portion of the target polynucleotide in the first direction with respect to the detector; and wherein the first portion at least partially overlaps with the second portion.

32. A method according to any one of claims 29 to 31, wherein the first portion is the same as the second portion.

33. A method according to any one of claims 29 to 32, wherein in step (iii) the distance the target polynucleotide moves with respect to the detector is at least 100 nucleotides in length.

34. A method according to any one of claims 29 to 33, wherein the detector is comprised in a structure having a first opening and a second opening, or comprises a transmembrane nanopore having a first opening and a second opening; and step (i) comprises contracting the first opening with the target polynucleotide.

35. A method according to claim 34, wherein (i) the motor protein controls the movement of the target polynucleotide in the direction from the second opening to the first opening; and (ii) when the target polynucleotide is unbound from the polynucleotide binding site of the motor protein, the target polynucleotide moves in the direction from the first opening to the second opening.

36. A method according to any one of claims 29 to 35, comprising applying a force across the detector, and wherein the motor protein controls the movement of the target polynucleotide with respect to the detector in the direction opposite to the applied force.

37. A method according to any one of claims 29 to 36, wherein the detector comprises a transmembrane nanopore spanning a membrane having a cis side and a trans side, and:
(i) the first opening of the nanopore is at the cis side of the membrane and the second opening of the nanopore is at the trans side; the motor protein controls the movement of the target polynucleotide through the nanopore from the trans side to the cis side of the membrane; and when the target polynucleotide is unbound from the polynucleotide binding site of the motor protein, the target polynucleotide moves through the nanopore from the cis side to the trans side of the membrane; or
(ii) the first opening of the nanopore is at the trans side of the membrane and the second opening of the nanopore is at the cis side; the motor protein controls the movement of the target polynucleotide through the nanopore from the cis side to the trans side of the membrane; and when the target polynucleotide is unbound from the polynucleotide binding site of the motor protein, the target polynucleotide moves through the nanopore from the trans side to the cis side of the membrane

38. A method according to any one of claims 29 to 37, wherein the target polynucleotide is attached to or comprises a leader configured to promote unbinding of the polynucleotide binding site of the motor protein from the target polynucleotide in the vicinity of the leader.

39. A method according to claim 38, wherein the target polynucleotide unbinds from the polynucleotide binding site of the motor protein when the motor protein contacts the leader.

40. A method according to claim 38 or claim 39, wherein the motor protein has a lower affinity for the leader than for the nucleotides of the target polynucleotide.

41. A method according to any one of claims 38 to 40, wherein the leader comprises a different type of nucleotide to the target polynucleotide.

42. A method according to any one of claims 38 to 41, wherein (i) the target polynucleotide comprises deoxyribonucleotides (DNA) and the leader comprises one or more nucleotides lacking both nucleobase and sugar moieties (spacer moieties), ribonucleotides (RNA), peptide nucleotides (PNA), glycerol nucleotides (GNA), threose nucleotides (TNA), locked nucleotides (LNA), bridged nucleotides (BNA), abasic nucleotides or nucleotides having a modified phosphate linkage; or (ii) the target polynucleotide comprises ribonucleotides (RNA) and the leader comprises one or more nucleotides lacking both nucleobase and sugar moieties (spacer moities), deoxyribonucleotides (DNA), peptide nucleotides (PNA), glycerol nucleotides (GNA), threose nucleotides (TNA), locked nucleotides (LNA), bridged nucleotides (BNA), abasic nucleotides or nucleotides having a modified phosphate linkage.

43. A method according to any one of claims 38 to 42, wherein the target polynucleotide comprises deoxyribonucleotides (DNA) and the leader comprises one or more spacer moieties and/or one or more ribonucleotides.

44. A method according to any one of claims 29 to 43, wherein the target polynucleotide does not disengage from the motor protein.

45. A method according to any one of claims 29 to 44, wherein the motor protein is modified to prevent the target polynucleotide disengaging from the target polynucleotide.

46. A method according to any one of claims 29 to 45, wherein the motor protein is modified to promote unbinding of the target polynucleotide from the polynucleotide binding site of the motor protein and/or to retard re-binding of the target polynucleotide to the polynucleotide binding site of the motor protein.

47. A method according to claim any one of claims 29 to 46, wherein the motor protein is modified with a closing moiety for (i) topologically closing the polynucleotide binding site of the motor protein around the target polynucleotide and (ii) promoting unbinding of the target polynucleotide from the polynucleotide binding site of the motor protein and/or retarding re-binding of the target polynucleotide to the polynucleotide binding site of the motor protein

48. A method according to claim 47, wherein the motor protein is modified to facilitate attachment of the closing moiety to the motor protein.

49. A method according to claim 48, wherein the motor protein is modified by substituting at least one amino acid in the motor protein for cysteine or for a non-natural amino acid.

50. A method according to any one of claims 47 to 49, wherein the closing moiety comprises a bifunctional crosslinker.

51. A method according to any one of claims 47 to 50, wherein the closing moiety crosslinks two amino acid residues of the motor protein, wherein at least one amino acid crosslinked by the closing moiety is a cysteine or a non-natural amino acid.

52. A method according to any one of claims 47 to 51, wherein the closing moiety has a length of from about 1 Å to about 100 Å.

53. A method according to any one of claims 47 to 49, wherein the closing moiety comprises a bond, preferably a disulphide bond.

54. A method according to any one of claims 47 to 52, wherein the closing moiety comprises a structure of formula [A-B-C], wherein A and C are each independently reactive functional groups for reacting with amino acid residues in the motor protein and B is a linking moiety.

55. A method according to claim 54, wherein A and C are each independently a cysteine-reactive functional group.

56. A method according to claim 54 or 55, wherein linking moiety B comprises a linear or branched, unsubstituted or substituted alkylene, alkenylene, alkynylene, arylene, heteroarylene, carbocyclylene or heterocyclylene moiety, which moiety is optionally interrupted by and/or terminated in one or more atoms or groups selected from O, N(R), S, C(O), C(O)NR, C(O)O, unsubstituted or substituted arylene, arylene-alkylene, heteroarylene, heteroarylene-alkylene, carbocyclylene, carbocyclylene-alkylene, heterocyclylene and heterocyclylene-alkylene; wherein R is selected from H, unsubstituted or substituted alkyl, and unsubstituted or substituted aryl.

57. A method according to any one of claims 54 to 56, wherein linking moiety B comprises an alkylene, oxyalkylene or polyoxyalkylene group and/or wherein A and C are each maleimide groups.

58. A method according to any one of claims 47 to 53 or 54 to 57, wherein the closing moiety has a length of from about 5 Å to about 50 Å.

59. A method according to any one of claims 29 to 58, comprising providing a condition for promoting the unbinding of the target polynucleotide from the polynucleotide binding site of the motor protein and/or for retarding re-binding of the target polynucleotide to the polynucleotide binding site of the motor protein.

60. A method according to claim 59, wherein providing said condition comprises increasing the temperature so as to increase the rate of unbinding of the target polynucleotide from the polynucleotide binding site of the motor protein.

61. A method according to claim 59 or 60, wherein providing said condition comprises increasing the temperature so as to reduce the rate of re-binding of the target polynucleotide to the polynucleotide binding site of the motor protein.

62. A method according to any one of claims 29 to 61, wherein the motor protein is a helicase.


SEQUENCE LISTING (72 sequences)

SEQ ID NO: 1 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 2 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 3 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 4 - protein, 226 aa, Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 5 - protein, 608 aa, Bacillus subtilis
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 6 - protein, 970 aa, Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 7 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 8 - protein, 439 aa, Artificial Sequence
  Dda helicase
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9-19 - skipped (11 entries)

SEQ ID NO: 20 - DNA, 3595 bp, Artificial Sequence
  Bacteriophage lambda fragment
gccatcagattgtgtttgttagtcgctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagcatgtagga

SEQ ID NO: 21 - DNA, 38 bp, Artificial Sequence
  Y-adapter oligo
  misc_feature (1)..(1): 5′ biotin, via TEG linker
  misc_feature (10)..(11): iSp18
ttttttttttaatgtacttcgttcagttacgtattgct

SEQ ID NO: 22 - DNA, 65 bp, Artificial Sequence
  Y-adapter oligo
  misc_feature (1)..(1): 5′ phosphate
  misc_feature (23)..(23): Bridged nucleic acid
  modified_base (24)..(24): m5c
  misc_feature (24)..(24): Bridged nucleic acid
  misc_feature (25)..(25): Bridged nucleic acid
  misc_feature (26)..(26): Bridged nucleic acid
  misc_feature (27)..(27): Bridged nucleic acid
gcaatacgtaactgaacgaagtacatttttgaggcgagcggtcaatttttttttttttttttttt

SEQ ID NO: 23 - DNA, 60 bp, Artificial Sequence
  Hairpin oligo
  misc_feature (1)..(1): 5′ phosphate
tgcaatacgtaactgaacgaagtacattaatgtacttcgttcagttacgtattgcatcct

SEQ ID NO: 24 - DNA, 91 bp, Artificial Sequence
  Hairpin oligo
  misc_feature (1)..(1): 5′ phosphate
tgcaatacgtaactgaacgaagtacatttttttgaagatagagcgattttttttttttttttgtacttcgttcagttacgtattgcatcct

SEQ ID NO: 25 - DNA, 88 bp, Artificial Sequence
  Hairpin oligo
  misc_feature (1)..(1): 5′ phosphate
tgcaatacgtaactgaacgaagtacatttttttgaagatagagcgattttttttttttttttgtacttcgttcagttacgtattgcat

SEQ ID NO: 26 - DNA, 92 bp, Artificial Sequence
  Hairpin oligo
  misc_feature (1)..(1): 5′ phosphate
  misc_feature (52)..(52): 5′ fluorescein
  misc_feature (53)..(53): 5′ fluorescein
  misc_feature (54)..(54): 5′ fluorescein
tgcaatacgtaactgaacgaagtacatttttttgaagatagagcgatttttttttttttttttgtacttcgttcagttacgtattgcatcct

SEQ ID NO: 27 - DNA, 13 bp, Artificial Sequence
  Oligo
  misc_feature (1)..(1): Bridged nucleic acid
  modified_base (2)..(2): m5c
  misc_feature (2)..(2): Bridged nucleic acid
  misc_feature (3)..(3): Bridged nucleic acid
  modified_base (4)..(4): m5c
  misc_feature (4)..(4): Bridged nucleic acid
  misc_feature (5)..(5): Bridged nucleic acid
tcgctctatcttc

SEQ ID NO: 28 - DNA, 89 bp, Artificial Sequence
  Y-adapter oligo
  misc_feature (35)..(36): iSp18
gttattcaagacttctttaatacacttttttttttaatgtacttcgttcagttacgtattgctttggcgtctgcttgggtgtttaacct

SEQ ID NO: 29 - DNA, 61 bp, Artificial Sequence
  Y-adapter oligo
  misc_feature (1)..(1): 5′ phosphate
ggttaaacacccaagcagacgcctttgaggcgagcggtcaatttttttttttttttttttt

SEQ ID NO: 30 - DNA, 27 bp, Artificial Sequence
  Y-adapter oligo
  misc_feature (23)..(23): Bridged nucleic acid
  modified_base (24)..(24): m5c
  misc_feature (24)..(24): Bridged nucleic acid
  misc_feature (25)..(25): Bridged nucleic acid
  misc_feature (26)..(26): Bridged nucleic acid
  misc_feature (27)..(27): Bridged nucleic acid
gcaatacgtaactgaacgaagtacatt

SEQ ID NO: 31 - DNA, 25 bp, Artificial Sequence
  Y-adapter oligo
  misc_feature (1)..(1): Bridged nucleic acid
  misc_feature (2)..(2): Bridged nucleic acid
  misc_feature (3)..(3): Bridged nucleic acid
  misc_feature (4)..(4): Bridged nucleic acid
  misc_feature (5)..(5): Bridged nucleic acid
  misc_feature (23)..(23): Bridged nucleic acid
  misc_feature (24)..(24): Bridged nucleic acid
  modified_base (25)..(25): m5c
  misc_feature (25)..(25): Bridged nucleic acid
gtgtattaaagaagtcttgaataac

SEQ ID NO: 32 - DNA, 25 bp, Artificial Sequence
  Y-adapter oligo
gtgtattaaagaagtcttgaataac

SEQ ID NO: 33 - DNA, 41 bp, Artificial Sequence
  C3 spacer oligo
  misc_feature (1)..(1): 5′ phosphate
  misc_feature (41)..(41): 30iSpC3
ggttaaacacccaagcagacgcctttgaggcgagcggtcaa

SEQ ID NO: 34-39 - skipped (6 entries)

SEQ ID NO: 40 - protein, 65 aa, Artificial Sequence
  HhH domain
GTGSGAWKEWLERKVGEGRARRLIEYFGSAGEVGKLVENAEVSKLLEVPGIGDEAVARLVPGGSS

SEQ ID NO: 41 - protein, 299 aa, Bacteriophage RB69
MFKRKSTADLAAQMAKLNGNKGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAASVADKVASDLDDFDKDMEAFSSAKTEDDFMSSSSSDDGDLDDLLAGL

SEQ ID NO: 42 - protein, 232 aa, Bacteriophage T7
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 43 - protein, 324 aa, Artificial Sequence
  UL42 processivity factor
MDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQKHHHHHH

SEQ ID NO: 44 - protein, 251 aa, Artificial Sequence
  Subunit 1 of PCNA
MFKIVYPNAKDFFSFINSITNVTDSIILNFTEDGIFSRHLTEDKVLMAIMRIPKDVLSEYSIDSPTSVKLDVSSVKKILSKASSKKATIELTETDSGLKIIIRDEKSGAKSTIYIKAEKGQVEQLTEPKVNLAVNFTTDESVLNVIAADVTLVGEEMRISTEEDKIKIEAGEEGKRYVAFLMKDKPLKELSIDTSASSSYSAEMFKDAVKGLRGFSAPTMVSFGENLPMKIDVEAVSGGHMIFWIAPRLLE

SEQ ID NO: 45 - protein, 245 aa, Artificial Sequence
  Subunit 2 of PCNA
MKAKVIDAVSFSYILRTVGDFLSEANFIVTKEGIRVSGIDPSRVVFLDIFLPSSYFEGFEVSQEKEIIGFKLEDVNDILKRVLKDDTLILSSNESKLTLTFDGEFTRSFELPLIQVESTQPPSVNLEFPFKAQLLTITFADIIDELSDLGEVLNIHSKENKLYFEVIGDLSTAKVELSTDNGTLLEASGADVSSSYGMEYVANTTKMRRASDSMELYFGSQIPLKLRFKLPQEGYGDFYIAPRAD

SEQ ID NO: 46 - protein, 246 aa, Artificial Sequence
  Subunit 3 of PCNA
MKVVYDDVRVLKDIIQALARLVDEAVLKFKQDSVELVALDRAHISLISVNLPREMFKEYDVNDEFKFGFNTQYLMKILKVAKRKEAIEIASESPDSVIINIIGSTNREFNVRNLEVSEQEIPEINLQFDISATISSDGFKSAISEVSTVTDNVVVEGHEDRILIKAEGESEVEVEFSKDTGGLQDLEFSKESKNSYSAEYLDDVLSLTKLSDYVKISFGNQKPLQLFFNMEGGGKVTYLLAPKVLE

SEQ ID NO: 47 - protein, 318 aa, Artificial Sequence
  UL42 processivity factor
TDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQK

SEQ ID NO: 48 - protein, 55 aa, Artificial Sequence
  (HhH)2 domain
WKEWLERKVGEGRARRLIEYFGSAGEVGKLVENAEVSKLLEVPGIGDEAVARLVP

SEQ ID NO: 49 - protein, 107 aa, Artificial Sequence
  (HhH)2-(HhH)2 domain
WKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILG

SEQ ID NO: 50 - protein, 132 aa, Homo sapiens
ESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 51 - protein, 123 aa, Artificial Sequence
  p5 protein
ENTNIVKATFDTETLEGQIKIFNAQTGGGQSFKNLPDGTIIEANAIAQYKQVSDTYGDAKEETVTTIFAADGSLYSAISKTVAEAASDLIDLVTRHKLETFKVKVVQGTSSKGNVFFSLQLSL

SEQ ID NO: 52 - protein, 177 aa, Escherichia coli
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 53 - protein, 301 aa, Artificial Sequence
  ssb from bacteriophage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 54 - protein, 984 aa, Methanopyrus kandleri
MALVYDAEFVGSEREFEEERETFLKGVKAYDGVLATRYLMERSSSAKNDEELLELHQNFILLTGSYACSIDPTEDRYQNVIVRGVNFDERVQRLSTGGSPARYAIVYRRGWRAIAKALDIDEEDVPAIEVRAVKRNPLQPALYRILVRYGRVDLMPVTVDEVPPEMAGEFERLIERYDVPIDEKEERILEILRENPWTPHDEIARRLGLSVSEVEGEKDPESSGIYSLWSRVVVNIEYDERTAKRHVKRRDRLLEELYEHLEELSERYLRHPLTRRWIVEHKRDIMRRYLEQRIVECALKLQDRYGIREDVALCLARAFDGSISMIATTPYRTLKDVCPDLTLEEAKSVNRTLATLIDEHGLSPDAADELIEHFESIAGILATDLEEIERMYEEGRLSEEAYRAAVEIQLAELTKKEGVGRKTAERLLRAFGNPERVKQLAREFEIEKLASVEGVGERVLRSLVPGYASLISIRGIDRERAERLLKKYGGYSKVREAGVEELREDGLTDAQIRELKGLKTLESIVGDLEKADELKRKYGSASAVRRLPVEELRELGFSDDEIAEIKGIPKKLREAFDLETAAELYERYGSLKEIGRRLSYDDLLELGATPKAAAEIKGPEFKFLLNIEGVGPKLAERILEAVDYDLERLASLNPEELAEKVEGLGEELAERVVYAARERVESRRKSGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 55 - protein, 299 aa, Methanopyrus kandleri
SGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 56 - protein, 853 aa, Escherichia coli
MSAIENFDAHTPMMQQYLRLKAQHPEILLFYRMGDFYELFYDDAKRASQLLDISLTKRGASAGEPIPMAGIPYHAVENYLAKLVNQGESVAICEQIGDPATSKGPVERKVVRIVTPGTISDEALLQERQDNLLAAIWQDSKGFGYATLDISSGRFRLSEPADRETMAAELQRTNPAELLYAEDFAEMSLIEGRRGLRRRPLWEFEIDTARQQLNLQFGTRDLVGFGVENAPRGLCAAGCLLQYAKDTQRTTLPHIRSITMEREQDSIIMDAATRRNLEITQNLAGGAENTLASVLDCTVTPMGSRMLKRWLHMPVRDTRVLLERQQTIGALQDFTAGLQPVLRQVGDLERILARLALRTARPRDLARMRHAFQQLPELRAQLETVDSAPVQALREKMGEFAELRDLLERAIIDTPPVLVRDGGVIASGYNEELDEWRALADGATDYLERLEVRERERTGLDTLKVGFNAVHGYYIQISRGQSHLAPINYMRRQTLKNAERYIIPELKEYEDKVLTSKGKALALEKQLYEELFDLLLPHLEALQQSASALAELDVLVNLAERAYTLNYTCPTFIDKPGIRITEGRHPVVEQVLNEPFIANPLNLSPQRRMLIITGPNMGGKSTYMRQTALIALMAYIGSYVPAQKVEIGPIDRIFTRVGAADDLASGRSTFMVEMTETANILHNATEYSLVLMDEIGRGTSTYDGLSLAWACAENLANKIKALTLFATHYFELTQLPEKMEGVANVHLDALEHGDTIAFMHSVQDGAASKSYGLAVAALAGVPKEVIKRARQKLRELESISPNAAATQVDGTQMSLLSVPEETSPAVEALENLDPDSLTPRQALEWIYRLKSLV

SEQ ID NO: 57 - protein, 64 aa, Sulfolobus solfataricus
MATVKFKYKGEEKEVDISKIKKVWRVGKMISFTYDEGGGKTGRGAVSEKDAPKELLQMLEKQKK

SEQ ID NO: 58 - protein, 99 aa, Sulfolobus solfataricus P2
EKMSSGTPTPSNVVLIGKKPVMNYVLAALTLLNQGVSEIVIKARGRAISKAVDTVEIVRNRFLPDKIEIKEIRVGSQVVTSQDGRQSRVSTIEIAIRKK

SEQ ID NO: 59 - protein, 88 aa, Sulfolobus solfataricus P2
TEKLNEIVVRKTKNVEDHVLDVIVLFNQGIDEVILKGTGREISKAVDVYNSLKDRLGDGVQLVNVQTGSEVRDRRRISYILLRLKRVY

SEQ ID NO: 60 - protein, 107 aa, Escherichia coli
AQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 61 - protein, 237 aa, Enterobacteria phage lambda
MSTKKKPLTQEQLEDARRLKAIYEKKKNELGLSQESVADKMGMGQSGVGALFNGINALNAYNAALLAKILKVSVEEFSPSIAREIYEMYEAVSMQPSLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 62 - protein, 60 aa, Artificial Sequence
  Cren7
MSSGKKPVKVKTPAGKEAELVPEKVWALAPKGRKGVKIGLFKDPETGKYFRHKLPDDYPI

SEQ ID NO: 63 - protein, 136 aa, Homo sapiens [sequence omitted]

SEQ ID NO: 64 - protein, 89 aa, Enterobacteria phage T4
MAKKEMVEFDEAIHGEDLAKFIKEASDHKLKISGYNELIKDIRIRAKDELGVDGKMFNRLLALYHKDNRDVFEAETEEVVELYDTVFSK

SEQ ID NO: 65 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 66 - protein, 375 aa, Citromicrobium bathyomarinum JL354
MKATIERATLLRCLSHVQSVVERRNTIPILSNVLIDADAGGGVKVMATDLDLQVVETMTAASVESAGAITVSAHLLFDIARKLPDGSQVSLETADNRMVVKAGRSRFQLPTLPRDDFPVIVEGELPTSFELPARELAEMIDRTRFAISTEETRYYLNGIFLHVSDEARPVLKAAATDGHRLARYTLDRPEGAEGMPDVIVPRKAVGELRKLLEEALDSNVQIDLSASKIRFALGGEGGVVLTSKLIDGTFPDYSRVIPTGNDKLLRLDPKAFFQGVDRVATIATEKTRAVKMGLDEDKVTLSVTSPDNGTAAEEIAAEYKAEGFEIGFNANYLKDILGQIDSDTVELHLADAGAPTLIRRDENSPALYVLMPMRV

SEQ ID NO: 67 - DNA, 89 bp, Artificial Sequence; identical to SEQ ID NO: 28
  Oligo
  misc_feature (35)..(36): iSp9

SEQ ID NO: 68 - DNA, 43 bp, Artificial Sequence
  Oligo
gtgtattaaagaagtcttgaataactttgaggcgagcggtcaa

SEQ ID NO: 69 - DNA, 30 bp, Artificial Sequence
  Oligo
  misc_feature (26)..(26): Bridged nucleic acid
  modified_base (27)..(27): m5c
  misc_feature (27)..(27): Bridged nucleic acid
  misc_feature (28)..(28): Bridged nucleic acid
  misc_feature (29)..(29): Bridged nucleic acid
  misc_feature (30)..(30): Bridged nucleic acid
tttgcaatacgtaactgaacgaagtacatt

SEQ ID NO: 70 - DNA, 26 bp, Artificial Sequence
  Leader oligo
  misc_feature (1)..(1): 5′ phosphate
  misc_feature (26)..(26): 30iSpC3
ggttaaacacccaagcagacgccttt

SEQ ID NO: 71 - DNA, 32 bp, Artificial Sequence
  Leader oligo
  misc_feature (1)..(1): 5′ phosphate
  modified_base (27)..(27): t
  modified_base (28)..(28): t
  modified_base (29)..(29): t
  modified_base (30)..(30): t
  modified_base (31)..(31): t
  misc_feature (32)..(32): 30iSpC3
  modified_base (32)..(32): t
ggttaaacacccaagcagacgcctttuuuuuu

SEQ ID NO: 72 - DNA, 25 bp, Artificial Sequence
  Leader oligo
  misc_feature (1)..(1): 5′ phosphate
  modified_base (24)..(24): t
  modified_base (25)..(25): t
  misc_feature (25)..(25): 30iSpC3
ggttaaacacccaagcagacgccuu